(19) United States
(12) Patent Application Publication
(10) Pub. No.: US 2023/0312740 A1
(43) Pub. Date: Oct. 5, 2023

(54) HUMANIZED CD38 AND ICAM1 ANTIBODIES AND USES THEREOF

(71) Applicant: Virtuoso BINco, Inc., San Mateo, CA (US)

(72) Inventor: Xiaocheng CHEN, San Mateo, CA (US)

(21) Appl. No.: 17/998,481

(22) PCT Filed: May 14, 2021
(86) PCT No.: PCT/US2021/032625

§ 371 Date: Nov. 10, 2022

Related U.S. Application Data
(60) Provisional application No. 63/024,931, filed on May 14, 2020.

(51) Int. Cl. C07K 16/28; A61P 35/00

(52) U.S. Cl. CPC C07K 16/2896; A61P 35/00; C07K 16/2821; C07K 2317/24; C07K 2317/31; C07K 2317/622; C07K 2317/732

(57) ABSTRACT

Disclosed herein are humanized anti-CD38 antibodies, humanized anti-ICAMl antibodies, and bispecific antibodies comprising humanized anti-CD38 and anti-ICAMl binding domains and methods of using humanized anti-CD38 antibodies, humanized anti-rCAMl antibodies, and bispecific antibodies comprising humanized anti-CD38 and anti-ICAMl binding domains for killing tumor cells and inhibiting tumor growth.

### 1. CROSS-REFERENCE

This application claims the benefit of U.S. Provisional Application No. 63/024,931, filed May 14, 2020, which is entirely incorporated herein by reference for all purposes.

### 2. INCORPORATION BY REFERENCE

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

### 2.1 SEQUENCE LISTING

[0002.1] The instant application contains a Sequence Listing which has been submitted electronically in ASCII format and is hereby incorporated by reference in its entirety. Said ASCII copy, created on May 14, 2021, is named 55429-707_601_SL.txt and is 143,044 bytes in size.

### 3. SUMMARY OF THE DISCLOSURE

This disclosure describes humanized bispecific antibodies that bind to the extracellular domains of CD38 and ICAM1. In the various embodiments disclosed herein, humanized bispecific antibodies that bind to the extracellular domains of CD38 and ICAM1 have cytotoxic and antitumor activity on a wide range of transformed cells with varying levels of CD38 and ICAM1 expression.

In one embodiment, a bispecific antibody comprises one or more humanized CD38 binding domains, one or more humanized ICAM1 binding domains, and a human Fc domain. In some embodiments, said humanized CD38 binding domain comprises an scFv. In some embodiments, said humanized CD38 binding domain comprises a variable domain of an IgG heavy chain and a variable domain of an IgG light chain. In some embodiments, said humanized CD38 binding domain comprises a heavy chain variable region and a light chain variable region. In some embodiments, said humanized ICAM1 binding domain comprises an scFv. In some embodiments, said humanized ICAM1 binding domain comprises a variable domain of an IgG heavy chain and a variable domain of an IgG light chain. In some embodiments, said humanized ICAM1 binding domain comprises a heavy chain variable region and a light chain variable region. In some embodiments, said isotype of the bispecific antibody is IgG1. In some embodiments, said human Fc domain is a heterodimeric Fc domain, wherein the heterodimeric Fc region comprises a knob chain and a hole chain, forming a knob-in-hole (KiH) structure. In a specific embodiment, said knob chain comprises mutation T366W and said hole chain comprises mutations T366S, L368A, and Y407V, wherein amino acid position numbering is according to the EU index of Kabat et al. In another embodiment, said knob chain comprises mutations S354C, T366W and the hole chain comprises mutations Y349C, T366S, L368A, and Y407V, wherein amino acid position numbering is according to the EU index of Kabat et al. In some embodiments, said Fc domain is afucosylated. In some embodiments, said Fc domain comprises one or more amino acid substitutions that enhance antibody-dependent cellular cytotoxicity (ADCC) activity. In a specific embodiment, said Fc domain comprises S239D, I332E, and A330L amino acid substitutions, and wherein the amino acid numbering is according to the EU index in Kabat et al. In some embodiments, the humanized CD38 binding domain comprises the variable domain of an anti-CD38 IgG, and the ICAM1 binding domain comprises an anti-ICAM1 single chain variable fragment (anti-ICAM1 scFv). In some embodiments, said humanized ICAM1 binding domain comprises the variable domain of an anti-ICAM1 IgG, and the CD38 binding domain comprises an anti-CD38 single chain variable fragment (anti-CD38 scFv). In some embodiments, the bispecific antibody further comprises a CH1 IgG domain and a CL IgG domain. In some embodiments, the bispecific antibody further comprises a T cell receptor (TCR) constant region, wherein the TCR constant region comprises a TCR alpha constant domain and a TCR beta constant domain. In a specific embodiment, said humanized CD38 binding domain comprises a VH-CD38 domain and a VL-CD38 domain, wherein the VH-CD38 domain is fused to the TCR beta constant domain, and the VL-CD38 domain is fused to the TCR alpha constant domain. In another embodiment, said humanized CD38 binding domain comprises a VH-CD38 domain and a VL-CD38 domain, wherein the VH-CD38 domain is fused to the CH1 IgG domain, and the VL-CD38 domain is fused to the CL IgG domain. In some embodiments, said humanized ICAM1 binding domain comprises a VH-ICAM1 domain and a VL-ICAM1 domain, wherein the VH-ICAM1 domain is fused to the TCR beta constant domain, and the VL-ICAM1 domain is fused to the TCR alpha constant domain. In some embodiments, said humanized ICAM1 binding domain comprises a VH-ICAM1 domain and a VL-ICAM1 domain, wherein the VH-ICAM1 domain is fused to the CH1 IgG domain, and the VL-ICAM1 domain is fused to the CL IgG domain. In some embodiments, said bi specific antibody exhibits a melting transition temperature of at least 55° C., at least 60° C. or at least 65° C.

In some embodiments, the humanized CD38 binding domain comprises a sequence at least 90% identical to SEQ ID NO: 40. In some embodiments, the humanized CD38 binding domain comprises a sequence at least 90% identical to SEQ ID NO: 41. In some embodiments, said humanized CD38 binding domain comprises a sequence at least 90% identical to SEQ ID NO: 35. In some embodiments, the humanized CD38 binding domain comprises sequences at least 90% identical to SEQ ID NO: 50, SEQ ID NO: 51, SEQ ID NO: 52, SEQ ID NO: 53, SEQ ID NO: 54, and SEQ ID NO: 55. In some embodiments, the HC CDR3 domain comprises glutamic acid 95, glutamic acid 100b, glycine 100c, and tyrosine 100d, and the LC CDR3 domain comprises glycine 90, tyrosine 91, serine 93, glycine 94, and tyrosine 96, wherein amino acid position numbering is according to the EU index of Kabat et al. In some embodiments, the humanized ICAM1 binding domain comprises a sequence at least 90% identical to SEQ ID NO: 44. In some embodiments, the humanized ICAM1 binding domain comprises a sequence at least 90% identical to SEQ ID NO: 45. In some embodiments, the humanized ICAM1 binding domain comprises sequences at least 90% identical to SEQ ID NO: 56, SEQ ID NO: 57, SEQ ID NO: 58, SEQ ID NO: 59, SEQ ID NO: 60, and SEQ ID NO: 61.

In some embodiments, the humanized CD38 binding domain is capable of binding to the extracellular domain of human CD38 with an equilibrium dissociation constant (KD or K_{D}) of less than 10 nM or less than 5 nM. In some embodiments, the humanized CD38 binding domain is capable of binding to the extracellular domain of human CD38 with an equilibrium dissociation constant (KD or K_{D}) of between 0.1 nM and 20 nM, between 0.5 nM and 15 nM, between 1 nM and 10 nM, or between 1 nM and 5 nM. In some embodiments, the humanized CD38 binding domain is capable of binding to the extracellular domain of human CD38 with an equilibrium dissociation constant (KD or K_{D}) of between 2 nM and 5 nM. In some embodiments, the humanized ICAM1 binding domain is capable of binding to the extracellular domain of human ICAM1 with an equilibrium dissociation constant (KD or K_{D}) of less than 1 nM, less than 0.5 nM, or less than 0.2 nM. In some embodiments, the humanized ICAM1 binding domain is capable of binding to the extracellular domain of human ICAM1 with an equilibrium dissociation constant (KD or K_{D}) of between 0.02 nM and 10 nM, between 0.05 nM and 5 nM, between 0.05 nM and 1 nM, or between 0.1 nM and 0.5 nM. In some embodiments, the humanized ICAM1 binding domain is capable of binding to the extracellular domain of human ICAM1 with an equilibrium dissociation constant (KD or K_{D}) of between 0.1 nM and 0.15 nM. In some embodiments, the KD is determined by surface plasmon resonance. In some embodiments, the bispecific antibody comprises an afucosylated Fc domain. In some embodiments, the bispecific antibody is capable of inducing an enhanced antigen-dependent cellular cytotoxicity (ADCC) effect on a target cell compared to an ADCC effect induced on the target cell by a an otherwise identical bispecific antibody that does not comprise an afucosylated Fc domain. In some embodiments, the bispecific antibody is capable of inducing an enhanced ADCC effect on a target cell compared to an ADCC effect induced on the target cell by a monospecific protein that comprises the humanized CD38 binding domain or the humanized ICAM1 binding domain. In some embodiments, the bispecific antibody is capable of inducing complement-dependent cytotoxicity on Daudi cells with EC50s (half maximal effective concentration(s)) of less than 10 nM or between 0.5 nM and 1.0 nM.

In one embodiment, a humanized anti-ICAM1 antibody or ICAM1-binding fragment thereof, comprises CDR sequences at least 90% identical to SEQ ID NO: 56, SEQ ID NO: 57, SEQ ID NO: 58, SEQ ID NO: 59, SEQ ID NO: 60, and SEQ ID NO: 61. In some embodiments, the humanized anti-ICAM1 antibody or ICAM1-binding fragment thereof of claim 42, comprising sequences at least 90% identical to SEQ ID NO: 44 and SEQ ID NO: 45. In some embodiments, said anti-ICAM1 antibody or ICAM1-binding fragment thereof comprises one or more ICAM1 binding domains that is capable of binding to the extracellular domain of human ICAM1 with a n equilibrium dissociation constant (KD or K_{D}) of less than 1 nM or less than 0.5 nM. In some embodiments, said humanized anti-ICAM1 antibody or ICAM1-binding fragment thereof comprises one or more ICAM1 binding domains that is capable of binding to the extracellular domain of human ICAM1 with a n equilibrium dissociation constant (KD or K_{D}) of between 0.02 nM and 10 nM, between 0.05 nM and 5 nM, or between 0.05 nM and 1 nM. In some embodiments, said humanized anti-ICAM1 antibody or ICAM1-binding fragment thereof comprises one or more ICAM1 binding domains that is capable of binding to the extracellular domain of human ICAM1 with a n equilibrium dissociation constant (KD or K_{D}) of between 0.1 nM and 0.5 nM. In some embodiments, said KD is determined by surface plasmon resonance.

In one embodiment, a pharmaceutical composition comprises the bispecific antibody of any one of the previous embodiments, the humanized anti-ICAM1 antibody, ICAM1-binding fragment thereof of any one of the previous embodiments, or any combinations thereof. In another embodiment, said pharmaceutical composition further comprises a pharmaceutically acceptable carrier, an excipient, or any combinations thereof.

In one embodiment, a method of killing a cell in a subject comprises administering to the subject the bispecific antibody of any one of the previous embodiments, or the humanized anti-ICAM1 antibody or ICAM1-binding fragment thereof of any of the previous embodiments, or said pharmaceutical composition, wherein the cell expresses CD38 and ICAM1. In some embodiments, the cell is lysed. In some embodiments, said cell is a tumor cell. In another embodiment, a method of reducing growth of a tumor in a subject comprises administering to the subject the bispecific antibody of any of the previous embodiments, or the humanized anti-ICAM1 antibody or ICAM1-binding fragment thereof of any one of the previous embodiments, or said pharmaceutical composition, wherein the tumor comprises a cell that expresses CD38 and ICAM1. In another embodiment, a method of treating a cancer in a subject comprising administering to the subject the bispecific antibody of any one of the previous embodiments, or the humanized anti-ICAM1 antibody or ICAM1-binding fragment thereof of any one of the previous embodiments, or said pharmaceutical composition, wherein the cancer comprises a cell that expresses CD38 and ICAM1. In some embodiments, said cancer comprises a solid tumor or a hematological malignancy. In some embodiments, said cancer comprises the hematological malignancy. In some embodiments, said hematological malignancy is multiple myeloma, lymphoma, or Burkitt lymphoma. In some embodiments, said cancer is a lung cancer or a prostate cancer. In some embodiments, said cell expresses at least as much ICAM1 on its surface as an NCI-H2291 cell.

In some embodiments, said cell expresses at least as much CD38 on its surface as an NCI-H2342 cell. In some embodiments, said cell expresses less CD38 on its surface than a Daudi cell. In some embodiments, the amount of CD38 on the surface of the cell is less than or equal to the amount of CD38 on the surface of a Raji cell. In some embodiments, the ratio of ICAM1 to CD38 on the surface of the cell is greater than the ratio of ICAM1 to CD38 on the surface of a Daudi cell. In some embodiments, the ratio of ICAM1 to CD38 on the surface of the cell is greater than or equal to the ratio of ICAM1 to CD38 on the surface of a Raji cell. In some embodiments, the cell expresses at least 5000, 10000, 150000, 20000, 30000, 50000, 100000, 150000, 200000, 250000, 300000, 400000, or 500000 ICAM1 proteins on its surface. In some embodiments, the cell expresses at least 50,000 ICAM1 proteins on its surface. In some embodiments, the cell expresses at least 100, 200, 300, 400, 500, 1000, 2000, 3000, 4000, or 5000 CD38 proteins on its surface. In some embodiments, the cell expresses at least 300 CD38 proteins on its surface. In some embodiments, the cell expresses less than about 350000, 300000, 250000, 200000, 150000, 100000, 50000, 30000, 20000, 15000, 10000, or 5000 CD38 proteins on its surface. In some embodiments, the cell expresses less than about 350,000 CD38 proteins on its surface. In some embodiments, the ratio of ICAM1 to CD38 on the surface of the cell is at least about 1, 1.5, 2.0, 2.5, 5, 10, 15, 20, 50, 100, or 200. In some embodiments, the ratio of ICAM1 to CD38 on the surface of the cell is at least about 1. In some embodiments, the ratio of ICAM1 to CD38 on the surface of the cell is at least about 10. In some embodiments, the subject is a human.

In another embodiment, a kit comprises the bispecific antibody of any one of the previous embodiments, or the humanized anti-ICAM1 antibody or ICAM1-binding fragment thereof of any one of the previous embodiments, or the pharmaceutical composition of the previous embodiments.

### 4. BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1B show amino acid sequence alignments of the VL (FIG. 1A) and VH (FIG. 1B) domains of chimeric and humanized variants of 18E4. The CDRs are highlighted in bold. Vertical lines denote conserved residues. Figures disclose SEQ ID NOS 6, 63-68, 41 and 69-77, respectively, in order of appearance.

FIGS. 2A-2B show amino acid sequence alignments of alanine scan variants of the h18E4-19 light chain (FIG. 2A) and heavy chain (FIG. 2B). Figures disclose SEQ ID NOS 41, 78-86, 77 and 87-99, respectively, in order of appearance.

FIGS. 3A-3B show amino acid sequence alignments of the VL (FIG. 3A) and VH (FIG. 3B) domains of chimeric and humanized variants of 8B12. The CDRs are noted in bold. Vertical lines denote conserved residues. Figures disclose SEQ ID NOS 26 and 100-111, respectively, in order of appearance.

FIGS. 4A-4B show amino acid sequence alignments of the VH CDRs (FIG. 4A) and LC CDRs (FIG. 4B) from anti-ICAM1 clones 8B12 and 11F2. Vertical lines denote conserved residues.

FIGS. 5A-5B show amino acid sequence alignments of the VL (FIG. 5A) and VH (FIG. 5B) domains of chimeric and humanized variants of 11F2. The CDRs are noted in bold. Vertical lines denote conserved residues. Figures disclose SEQ ID NOS 16, 112-113, 45, 114-117, 44 and 118-120, respectively, in order of appearance.

FIGS. 6A-6C show exemplary bispecific TCR-chimera antibody formats. (FIG. 6A) Asymmetric bivalent 1+1 (FIG. 6B) Asymmetric Trivalent 2+1 (FIG. 6C) Symmetric Tetravalent 2+2. Solid rectangles indicate IgG constant domains. Triangular extension and indentation indicate knob-into-hole mutations in Fc domains. Checkered ovals indicate TCR constant domains. Grey and White rectangles indicate different antigen-binding domains.

FIGS. 7A-7B show CD38xICAM1 bispecific in a TCR-chimera antibody format (FIG. 7A) and in the three chain antibody format (FIG. 7B). Solid rectangles indicate IgG constant domains. Triangular extension and indentation indicate knob-into-hole mutations in Fc domains. Checkered ovals indicate TCR constant domains. Grey and White rectangles indicate different antigen-binding domains. Weaved oval indicates an anti-CD38 scFv.

FIGS. 8A-8E show antibody-dependent cellular cytotoxicity (ADCC) mediated lysis of Daudi (FIG. 8A), Raji (FIG. 8B), HuNS1 (FIG. 8C), NCI-H2342 (FIG. 8D), and NCI-2291 (FIG. 8E) cells by bispecific or bivalent antibodies.

FIGS. 9A-9C show growth inhibition of Raji lymphoma (FIG. 9A), KMS-26 multiple myeloma (FIG. 9B), or HCC44 lung carcinoma (FIG. 9C) cell derived xenografts by bispecific or bivalent antibodies.

FIG. 10 shows growth inhibition of LY3071 lymphoma patient derived xenografts by bispecific or bivalent antibodies.

### 5. DETAILED DESCRIPTION OF THE DISCLOSURE

Antibodies that bind to CD38 are useful for the treatment of cancers that express CD38. Anti-CD38 antibodies are thought to kill cancer cells by various mechanisms including antibody-dependent cell-mediated cytotoxicity and complement-dependent cytotoxicity. One such antibody, daratumumab, is approved for the treatment of adults with multiple myeloma. Reduced CD38 expression can limit the efficacy of anti-CD38 antibodies. Proposals to overcome this limitation include treatment with an antibody having a higher affinity for CD38, treatment with an antibody that binds to a different epitope on CD38, treatment with an antibody that more effectively inhibits CD38 enzymatic activity, treatment with a tetravalent anti-CD38 antibody, and concurrent treatment with all-trans retinoic acid to increase CD38 expression.

Antibody-based therapeutics have emerged as effective cancer treatment options due to the specificity and affinity of the antibody to target binding and ease of chemical and molecular modifications. For example, approved antibody-based therapy includes monoclonal antibodies such as rituximab, tositumomab, and trastuzumab; and bispecific T-cell engager such as Blinatumomab.

In some instances, low receptor copy numbers on a target cell, or low affinity toward an antigen has hindered the therapeutic effect of an antibody-based therapy. In some cases, non-specificity of an antibody toward a target antigen or the presence of a target in both cancer and non-cancer cells have further limited the use of an antibody-based therapy.

CD38, also known as cyclic ADP ribose hydrolase, is a type II transmembrane glycoprotein with a long C-terminal extracellular domain and a short N-terminal cytoplasmic domain. CD38 mediates cytokine secretion and activation and proliferation of lymphocytes (Funaro et al, J Immunology 145:2390-6, 1990; Guse et al, Nature 398:70-3, 1999), and via its NAD glycohydrolase activity regulates extracellular NAD+ levels which have been implicated in modulating the regulatory T-cell compartment (Adriouch et al., 14: 1284-92, 2012; Chiarugi et al., Nature Reviews 12:741-52, 2012). In some instances, CD38 is upregulated in different types of cancer, in particular, in hematologic malignancies such as multiple myeloma.

ICAM1, also known as CD54, is an Ig-like cell adhesion molecule. ICAM1 is an endothelial- and leukocyte-associated transmembrane protein and is involved in stabilizing cell-cell interactions and facilitating leukocyte endothelial transmigration. ICAM1 is expressed in various cell types, including endothelial cells and leukocytes, and can be expressed or overexpressed in different cancer cells such as myeloma, pancreatic cancer, glioma, lung cancer, melanoma, colorectal cancer, and lymphoma.

In some embodiments, disclosed herein are humanized anti-CD38 antibodies, humanized anti-ICAM1 antibodies, and bispecific antibodies with humanized CD38 and ICAM1 binding domains. In additional embodiments, further described herein are methods of treating a cancer with use of an anti-CD38 antibody, an anti-ICAM1 antibody, or a multi-specific antibody (e.g., a bispecific CD38/ICAM1 antibody).

The term “bispecific antibody,” as used herein, generally refers to an antibody that binds specifically to at least two different antigens, and includes antibodies that can only bind specifically to two different antigens, and also includes antibodies that can bind specifically to two different antigens and further includes or is conjugated to one or more additional binding domains that bind specifically to a third, a fourth, or more antigens.

### 5.1 Humanized Anti-CD38 Antibodies

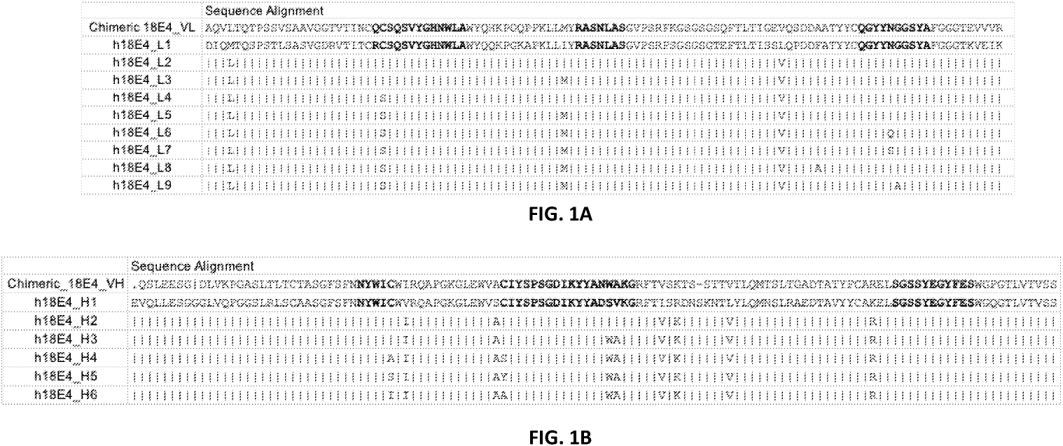

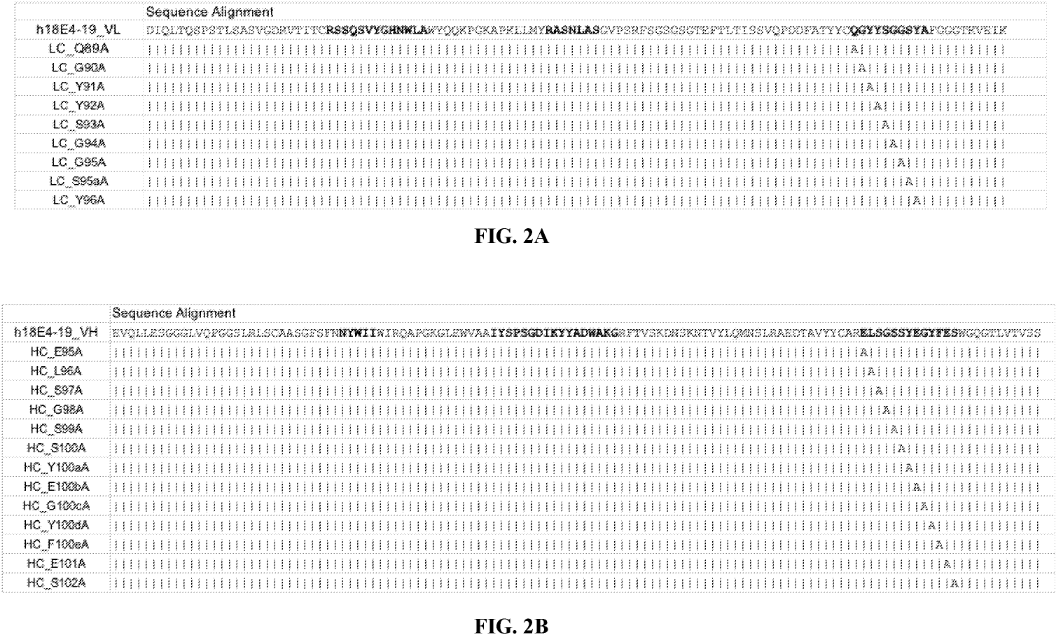

In certain embodiments, disclosed herein is a humanized anti-CD38 antibody. In some embodiments, a humanized anti-CD38 antibody described herein is a full-length antibody, comprising a heavy chain (HC) and a light chain (LC). In some cases, the HC comprises a sequence selected from FIG. 1B or FIG. 2B. In some cases, the LC comprises a sequence selected from FIG. 1A or FIG. 2A. In some instances, the anti-CD38 antibody comprises a variable heavy chain (VH) region and a variable light chain (VL) region, wherein the VH region comprises CDR1 sequence SEQ ID NO: 50; CDR2 sequence SEQ ID NO: 51; and CDR3 sequence SEQ ID NO: 52; and wherein the VL region comprises CDR1 sequence SEQ ID NO: 53; CDR2 sequence SEQ ID NO: 54; and CDR3 sequence SEQ ID NO: 55.

In some embodiments, the humanized anti-CD38 antibody comprises a VH region and a VL region in which the sequence of the VH region comprises about 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or 100% sequence identity to SEQ ID NOs: 5 or 40 and the sequence of the VL region comprises about 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or 100% sequence identity to SEQ ID NOs: 6 or 41.

In some embodiments, a humanized anti-CD38 antibody is a full-length antibody. In other embodiments, the humanized anti-CD38 antibody is a binding fragment. In some instances, the humanized anti-CD38 antibody comprises an antibody or binding fragment thereof, a monoclonal antibody or binding fragment thereof, a chimeric antibody or binding fragment thereof, or a humanized antibody or binding fragment thereof. In some cases, the humanized anti-CD38 antibody comprises a monovalent Fab, a divalent Fab′2, a single-chain variable fragment (scFv), or binding fragment thereof.

In some embodiments, a humanized anti-CD38 antibody described herein has an enhanced ADCC and/or CDC compared to daratumumab as a reference antibody. In some cases, the enhanced ADCC and/or complement-dependent cytotoxicity (CDC) is an increase of at least 10%, 20%, 30%, 40%, 50%, 60%, 70%, 80%, 90%, 100%, 150%, 200%, 300%, 400%, or more compared to daratumumab. In some cases, the enhanced ADCC and/or CDC is an increase of about 10%, 20%, 30%, 40%, 50%, 60%, 70%, 80%, 90%, 100%, 150%, 200%, 300%, 400%, or more compared to daratumumab.

In some cases, the enhanced ADCC and/or CDC is an increase of at least 1-fold, 2-fold, 3-fold, 4-fold, 5-fold, 6-fold, 7-fold, 8-fold, 9-fold, 10-fold, 15-fold, 20-fold, 30-fold, 40-fold, 50-fold, or more compared to reference antibody daratumumab. In some cases, the enhanced ADCC and/or CDC is an increase of about1-fold, 2-fold, 3-fold, 4-fold, 5-fold, 6-fold, 7-fold, 8-fold, 9-fold, 10-fold, 15-fold, 20-fold, 30-fold, 40-fold, 50-fold, or more compared to reference antibody daratumumab.

In some embodiments, a humanized anti-CD38 antibody described herein has a half maximal effective concentration (EC50 or EC₅₀) of from about 1 × 10⁻⁶ nM to about 2 nM, such as about 4 × 10⁻⁶ nM, about 0.000014 nM, 0.00007 nM, 0.00006 nM, about 0.00010 nM, about 0.0002 nM, about 0.0003 nM, 0.1 nM, about 0.2 nM, about 0.3 nM, about 0.4 nM, about 0.5 nM, about 0.6 nM, about 0.7 nM, about 0.8 nM, about 0.9 nM, about 1.0 nM, or from about 0.00001 to about 0.00003 nM in an in vitro cytotoxicity assay to determine ADCC activity, for instance, using PBMC effector cells and targeting cancer cells such as B-lymphoblast cells from a lymphoma, for example, Daudi cells.

In some embodiments, a humanized anti-CD38 antibody described herein has an improved cell killing effect compared to reference antibody daratumumab. In some cases, the improved cell killing effect is an increase of at least 10%, 20%, 30%, 40%, 50%, 60%, 70%, 80%, 90%, 100%, 150%, 200%, 300%, 400%, or more compared to reference antibody daratumumab. In some cases, the improved cell killing effect is an increase of about 10%, 20%, 30%, 40%, 50%, 60%, 70%, 80%, 90%, 100%, 150%, 200%, 300%, 400%, or more compared to reference antibody daratumumab.

In some instances, the improved cell killing effect is an increase of at least 1-fold, 2-fold, 3-fold, 4-fold, 5-fold, 6-fold, 7-fold, 8-fold, 9-fold, 10-fold, 15-fold, 20-fold, 30-fold, 40-fold, 50-fold, or more compared to reference antibody daratumumab. In some cases, the improved cell killing effect is an increase of about 1-fold, 2-fold, 3-fold, 4-fold, 5-fold, 6-fold, 7-fold, 8-fold, 9-fold, 10-fold, 15-fold, 20-fold, 30-fold, 40-fold, 50-fold, or more compared to reference antibody daratumumab.

In some embodiments, a humanized anti-CD38 antibody described herein has an improved serum half-life compared to reference antibody daratumumab. In some instances, the improved serum half-life is at least 30 minutes, 1 hour, 1.5 hours, 2 hours, 3 hours, 4 hours, 5 hours, 6 hours, 7 hours, 8 hours, 9 hours, 10 hours, 12 hours, 18 hours, 24 hours, 2 days, 3 days, 4 days, 5 days, 6 days, 7 days, 14 days, 30 days, or longer than reference antibody daratumumab.

In some cases, the serum half-life of an anti-CD38 antibody described herein is at least 30 minutes, 1 hour, 1.5 hours, 2 hours, 3 hours, 4 hours, 5 hours, 6 hours, 7 hours, 8 hours, 9 hours, 10 hours, 12 hours, 18 hours, 24 hours, 2 days, 3 days, 4 days, 5 days, 6 days, 7 days, 14 days, 30 days, or longer. In some cases, the serum half-life of an anti-CD38 antibody described herein is about 30 minutes, 1 hour, 1.5 hour, 2 hours, 3 hours, 4 hours, 5 hours, 6 hours, 7 hours, 8 hours, 9 hours, 10 hours, 12 hours, 18 hours, 24 hours, 2 days, 3 days, 4 days, 5 days, 6 days, 7 days, 14 days, 30 days, or longer.

### 5.2 Anti-ICAM1 Antibodies

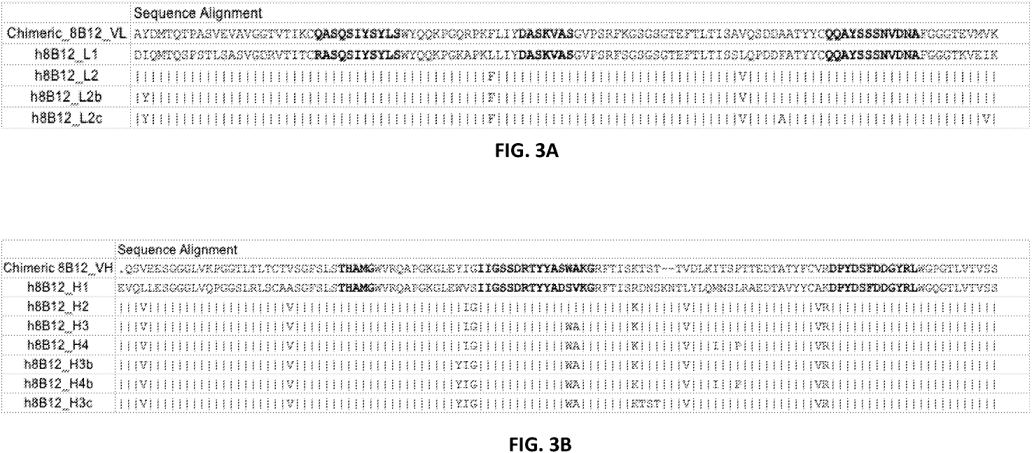

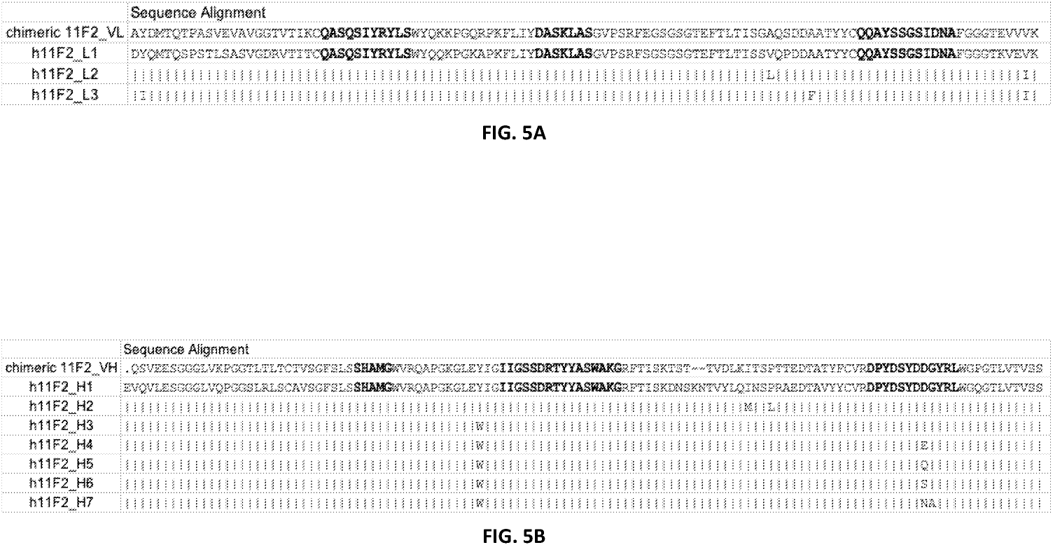

In certain embodiments, disclosed herein is a humanized anti-ICAM1 antibody. In some embodiments, a humanized anti-ICAM1 antibody described herein is a full-length antibody, comprising a heavy chain (HC) and a light chain (LC). In some cases, the HC comprises a sequence selected from FIG. 3B or FIG. 5B. In some cases, the LC comprises a sequence selected from FIG. 3A or FIG. 5A. In some embodiments, the anti-ICAM1 antibody comprises a VH region and a VL region, wherein the VH region comprises CDR1 sequence selected from SEQ ID NOs: 17, 27 and 56; CDR2 sequence selected from SEQ ID NOs: 18, 28, and 57; and CDR3 sequence selected from SEQ ID NOs: 19, 29, and 58; and wherein the VL region comprises CDR1 sequence selected from SEQ ID NOs: 20, 30, and 59; CDR2 sequence selected from SEQ ID NOs: 21, 31, and 60; and CDR3 sequence selected from SEQ ID NOs: 22, 30, and 61.

In some embodiments, the humanized anti-ICAM1 antibody comprises a VH region and a VL region in which the sequence of the VH region comprises about 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or 100% sequence identity to SEQ ID NOs: 44 and the sequence of the VL region comprises about 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or 100% sequence identity to SEQ ID NOs: 45.

In some embodiments, a humanized anti-ICAM1 antibody is a full-length antibody. In other embodiments, the anti-ICAM1 antibody is a binding fragment. In some instances, the anti-ICAM1 antibody comprises an antibody or binding fragment thereof, a monoclonal antibody or binding fragment thereof, a chimeric antibody or binding fragment thereof, or a humanized antibody or binding fragment thereof. In some cases, a humanized anti-ICAM1 antibody comprises a monovalent Fab, a divalent Fab′2, a single-chain variable fragment (scFv), or binding fragment thereof.

In some embodiments, a humanized anti-ICAM1 antibody described herein has a half maximal effective concentration (EC50 or EC₅₀) of from about 0.001 nM to about 2.5 nM, such as about 0.02 nM, 0.03 nM, about 0.04 nM, about 0.05 nM, about 0.06 nM, about 0.09 nM, about 0.1 nM, about 1.2 nM, about 1.7 nM, about 2.0 nM, about 2.5 nM, in an in vitro cytotoxicity assay to determine ADCC activity, for instance, using cancer cells such as human prostate cancer cells.

In some cases, the serum half-life of a humanized anti-ICAM1 antibody described herein is at least 30 minutes, 1 hour, 1.5 hours, 2 hours, 3 hours, 4 hours, 5 hours, 6 hours, 7 hours, 8 hours, 9 hours, 10 hours, 12 hours, 18 hours, 24 hours, 2 days, 3 days, 4 days, 5 days, 6 days, 7 days, 14 days, 30 days, or longer. In some cases, the serum half-life of an anti-ICAM1 antibody described herein is about 30 minutes, 1 hour, 1.5 hour, 2 hours, 3 hours, 4 hours, 5 hours, 6 hours, 7 hours, 8 hours, 9 hours, 10 hours, 12 hours, 18 hours, 24 hours, 2 days, 3 days, 4 days, 5 days, 6 days, 7 days, 14 days, 30 days, or longer.

### 5.3 Bispecific Antibodies

In certain embodiments, described herein is a humanized bispecific anti-CD38 and anti-ICAM1 antibody, or binding fragment thereof. In some instances, the humanized bispecific anti-CD38 and anti-ICAM1 antibody comprises a first humanized targeting moiety that specifically binds to CD38 and a second humanized targeting moiety that specifically binds to ICAM1. In some instances, the humanized bispecific antibody is bivalent, trivalent, tetravalent, or more than tetravalent. In some instances, the humanized bispecific antibody has more than one binding site that binds to CD38. In some instances, the bispecific antibody has more than one binding site that binds to ICAM1. In some instances, the humanized bispecific antibody or binding fragment thereof is a bispecific antibody conjugate, a hybrid bispecific IgG, a variable domain only bispecific antibody, a CH1/CL fusion protein, a Fab fusion protein, a non-immunoglobulin fusion protein, a Fc-modified IgG, an appended & Fc-modified IgG, a modified Fc and CH3 fusion protein, an appended IgG-HC fusion, a Fc fusion, a CH3 fusion, an IgE/IgM CH2 fusion, or a F(ab′)2 fusion.

In some embodiments, a humanized bispecific antibody described herein comprises an IgG framework, an IgA framework, an IgE framework, or an IgM framework. In some instances, the anti-CD38 antibody comprises an IgG framework (e.g., IgG1, IgG2, IgG3, or IgG4). In such instances, the humanized bispecific antibody comprises an IgG1, IgG2, IgG3, or an IgG4 framework.

In some cases, the bispecific antibody further comprises one or more mutations in a framework region, e.g., in the CH1 domain, CH2 domain, CH3 domain, hinge region, or a combination thereof. In some instances, the one or more mutations are to stabilize the antibody and/or to increase half-life. In some instances, the one or more mutations are to modulate Fc receptor interactions, to increase ADCC or complement-dependent cytotoxicity (CDC). In other instances, the one or more mutations are to reduce or eliminate Fc effector functions such as FcγR-binding, ADCC, or CDC. In additional instances, the one or more mutations are to modulate glycosylation, e.g. fucosylation. In some cases, the one or more mutations enhance stability, increase half-life, decrease glycosylation, and/or modulate Fc receptor interactions, e.g., to increase or decrease ADCC and/or CDC.

In some cases, the bispecific antibody comprises an IgG1 framework. In some embodiments, the constant region of the humanized anti-CD38 antibody is modified at one or more amino acid positions to alter Fc receptor interaction. Exemplary residues that modulate or alter Fc receptor interaction include, but are not limited to, G236, S239, T250, M252, S254, T256, K326, A330, I332, E333A, M428, H433, or N434 (Kabat numbering; EU index of Kabat et al 1991 Sequences of Proteins of Immunological Interest). In some instances, the mutation comprises G236A, S239D, T250Q, M252Y, S254T, T256E, K326W, A330L, I332E, E333A, E333S, M428L, H433K, or N434F.

In some embodiments, the modification at one or more amino acid positions in the IgG1 constant region to alter Fc receptor interaction leads to increased half-life. In some instances, the modification at one or more amino acid positions comprise T250, M252, S254, T256, M428, H433, N434, or a combination thereof; e.g., comprising T250Q/M428L or M252Y/S254T/T256E and H433K/N434F.

In some embodiments, a humanized bispecific antibody described above comprises a knobs-into-holes (KIH) format. In some cases, the KIH is located in the Fc region, in which the residues within the CH3 domain are optionally modified based on the disclosure of WO96/027011; Ridgway, et. al., Protein Eng. 9 (1996) 617-621; Merchant, et. al., Nat. Biotechnol. 16 (1998) 677-681; PCT/US19/61884; or Carter, J. Immunol. Protein Engineering 9(7) 617-621, 1996. In some cases, one member of a CH3 domain pair is the “knob” chain while the other is the “hole” chain, and additional disulfide bridges are optionally introduced to further stabilize the antibody and/or to increase yield.

In some instances, the humanized bispecific antibody is an IgG1, and the CH3 domain of the “knob” chain comprises a T366W mutation and the CH3 domain of the “hole” chain comprises mutations T366S, L368A, and Y407V. In some cases, the CH3 domain of the “knob” chain further comprises a Y349C mutation which forms an interchain disulfide bridge with either E356C or S354C in the CH3 domain of the “hole” chain.

In some instances, the CH3 domain of the “knob” chain comprises R409D and K370E mutations, and the CH3 domain of the “hole” chain comprises D399K and E357K. In some cases, the CH3 domain of the “knob” chain further comprises a T366W mutation, and the CH3 domain of the “hole” chain further comprises mutations T366S, L368A, and Y407V.

In some embodiments, the bispecific antibody and antigen-binding fragments thereof, comprise a heavy chain variable fragment (VH) of a first antibody fused to a first T-cell receptor (TCR) constant region, and a light chain variable domain (VL) of the first antibody fused to a second TCR constant region, wherein the first and second TCR constant regions are capable of forming a dimer, and the first antibody has the first antigenic specificity. In some instances, the TCR constant regions are the TCR alpha and TCR beta constant regions. In some embodiments, the VH-TCR fusion polypeptide is fused to a human IgG1 Fc domain. See WO 2019/057122

In some embodiments, the modification at one or more amino acid positions in the IgG1 constant region to alter Fc receptor interaction leads to increased ADCC and/or CDC. In some instances, the modification at one or more amino acid positions comprises S239, K326, A330, I332, E333, or a combination thereof. In some instances, the modification at one or more amino acid positions for increased ADCC and/or CDC comprises, e.g., E333A, S239D/A330L/I332E, or K326W/E333S. In some cases, the modification at one or more amino acid positions for increased ADCC comprises S239D/A330L/I332E. In some cases, the modification at one or more amino acid positions for increased CDC comprises K326W/E333S.

In some embodiments, the modification at one or more amino acid positions in the IgG1 constant region to alter Fc receptor interaction leads to increased macrophage phagocytosis. In some instances, the modification at one or more amino acid positions comprises G236, S239, I332, or a combination thereof. In some cases, the modification at one or more amino acid positions for increased macrophage phagocytosis comprises the combination of S239D/I332I/G236A.

In some embodiments, the IgG1 constant region is modified at amino acid N297 (Kabat numbering) in which residue N297 is afucosylated, wherein the oligosaccharides do not contain fucose sugar units.

In some embodiments, the bispecific antibody comprises an IgG2 framework. In some instances, one or more amino acid positions in the IgG2 framework are modified to alter Fc receptor interaction, e.g., to increase ADCC and/or CDC. In some cases, one or more amino acid positions in the IgG2 framework are modified to stabilize the antibody and/or to increase half-life. In some instances, one or more amino acid positions in the IgG2 framework are modified to modulate glycosylation. In some cases, the IgG2 constant region is afucosylated.

In some embodiments, the bispecific antibody comprises an IgG3 framework. In some instances, one or more amino acid positions in the IgG3 framework are modified to alter Fc receptor interaction, e.g., to increase ADCC and/or CDC. In some cases, one or more amino acid positions in the IgG3 framework are modified to stabilize the antibody and/or to increase half-life. In some instances, one or more amino acid positions in the IgG3 framework are modified to modulate glycosylation. In some cases, the constant region of the antibody is modified at amino acid R435 to extend the half-life, e.g., R435H (Kabat numbering). In some instances, the constant region is afucosylated at residue N297.

In some embodiments, the bispecific antibody comprises an IgG4 framework. In some instances, one or more amino acid positions in the IgG4 framework are modified to alter Fc receptor interaction, e.g., to increase ADCC and/or CDC. For example, mutations to increase ADCC comprises, in some embodiments, S239D, I332E, and A330L (amino acid numbering is according to the EU index in Kabat et al), such as described in U.S. Pat. No. 8,093,359. In some cases, one or more amino acid positions in the IgG4 framework are modified to stabilize the antibody and/or to increase half-life. In some instances, one or more amino acid positions in the IgG4 framework are modified to modulate glycosylation. In some cases, the constant region is modified at a hinge region to prevent or reduce strand exchange. In some instances, the amino acid that is modified is S228 (e.g., S228P).

In some embodiments, the human IgG constant region is modified to ADCC and/or CDC, e.g., with an amino acid modification described in Natsume et al., 2008 Cancer Res, 68(10): 3863-72; Idusogie et al., 2001 J Immunol, 166(4): 2571-5; Moore et al., 2010 mAbs, 2(2): 181- 189; Lazar et al., 2006 PNAS, 103(11): 4005-4010, Shields et al., 2001 JBC, 276( 9): 6591- 6604; Stavenhagen et al., 2007 Cancer Res, 67(18): 8882-8890; Stavenhagen et al., 2008 Advan. Enzyme Regul., 48: 152-164; Alegre et al, 1992 J Immunol, 148: 3461-3468; Reviewed in Kaneko and Niwa, 2011 Biodrugs, 25(1): 1-11.

In some embodiments, the human IgG constant region is modified to induce heterodimerization. For example, having an amino acid modification within the CH3 domain at Thr366, which when replaced with a more bulky amino acid, e.g., Trp (T366W), is able to preferentially pair with a second CH3 domain having amino acid modifications to less bulky amino acids at positions Thr366, Leu368, and Tyr407, e.g., Ser, Ala, and Val, respectively (T366S/L368A/Y407V). In some cases, heterodimerization via CH3 modifications is further stabilized by the introduction of a disulfide bond, for example by changing Ser354 to Cys (S354C) and Y349 to Cys (Y349C) on opposite CH3 domains (Reviewed in Carter, 2001 Journal of Immunological Methods, 248: 7-15).

In some instances, a humanized bispecific antibody described herein has reduced or lacks glycosylation but is not modified at amino acid Asn297 (Kabat numbering). In these instances, the glycosylation is, for example, eliminated by production of the antibody in a host cell that lacks a post-translational glycosylation capacity, for example a bacterial or yeast derived system or a modified mammalian cell expression system. In certain aspects, such a system is a cell-free expression system.

In some embodiments, the multispecific protein comprising a CD38-binding first component and an ICAM1-binding second component have different affinities (KD) for their respective target antigens as measured by surface plasmon resonance.

In some cases, the first component binds to human CD38 with a KD of from about 0.1 nM to about 100 nM, from about 0.15 nM to about 95 nM, from about 0.2 nM to about 90 nM, from 0.25 nM to about 85 nM, from about 0.3 nM to about 80 nM, from about 0.35 nM to about 75 nM, from about 0.4 nM to about 70 nM, from about 0.5 nM to about 70 nM, from about 0.6 nM to about 60 nM, from about 0.7 nM to about 50 nM, from about 0.8 nM to about 40 nM, from about 0.9 nM to about 30 nM, from about 1 nM to about 20 nM, from about 1.5 nM to about 10 nM, from about 2 nM to about 5 nM, from about 0.01 nM to about 25 nM, from about 0.01 nM to about 20 nM, from about 0.01 nM to about 10 nM, from about 0.01 nM to about 5 nM, from about 0.02 nM to about 20 nM, from about 0.04 nM to about 20 nM, from about 0.06 nM to about 20 nM, from about 0.08 nM to about 20 nM, or from about 0.1 nM to about 20 nM,.

In some cases, the second component binds to human ICAM1 with a KD of about 0.1 nM to about 100 nM, from about 0.15 nM to about 95 nM, from about 0.2 nM to about 90 nM, from 0.25 nM to about 85 nM, from about 0.3 nM to about 80 nM, from about 0.35 nM to about 75 nM, from about 0.4 nM to about 70 nM, from about 0.5 nM to about 70 nM, from about 0.6 nM to about 60 nM, from about 0.7 nM to about 50 nM, from about 0.8 nM to about 40 nM, from about 0.9 nM to about 30 nM, from about 1 nM to about 20 nM, from about 1.5 nM to about 10 nM, from about 0.15 nM to about 30 nM, from about 0.16 nM to about 25 nM, from about 0.1 nM to about 0.15 nM, from about 0.05 nM to about 0.15 nM, from about 0.1 nM to about 0.25 nM, from about 0.05 nM to about 0.25 nM, from about 0.17 nM to about 20 nM, from 0.18 nM to about 15 nM, from about 0.19 nM to about 10 nM, from about 0.1 nM to about 6 nM, from about 0.2 nM to about 6 nM, from about 0.2 nM to about 4 nM, from about 0.2 nM to about 2 nM, from about 0.2 nM to about 1.5 nM, from about 0.2 nM to about 1 nM, from about 0.2 nM to about 0.8 nM, from about 0.2 nM to about 0.6 nM, or from about 0.2 nM to about 0.4 nM.

### 5.4 Humanization of Bispecific Antibodies and Their Binding Components

In some embodiments, bispecific antibodies, and binding fragments thereof, are derived from non-human (e.g., rabbit) antibodies. In some instances, the humanized form of the non-human antibody contains the minimal non-human sequence to maintain original antigenic specificity. In some cases, the humanized antibodies are human immunoglobulins (acceptor antibody), wherein the CDRs of the acceptor antibody are replaced by residues of the CDRs of a non-human immunoglobulin (donor antibody), such as rat, rabbit, mouse, having the desired specificity, affinity, and capacity. In some instances, the framework region (FR) residues of the human immunoglobulin are replaced by corresponding non-human residues of the donor antibody.

### 5.5 Bispecific Antibody Binding to Target Cells

In some embodiments, the humanized bispecific antibody of the present disclosure that comprises a first component that binds CD38 and a second component that binds ICAM1 binds to a cell that expresses on its surface target antigens of the bispecific protein, with at least 2-50 fold, 10-100 fold, 2-fold, 5-fold, 10-fold, 25-fold, 50-fold or 100-fold, or 20-50%, 50-100%, 20%, 25%, 30%, 40%, 50%, 60%, 70%, 80%, 90% or 100% or higher affinity (e.g., preferentially binds) compared to the binding affinity of an antibody that is monospecific to only one of CD38 or ICAM1, to the cell.

In some embodiments, a humanized bispecific antibody provided herein binds to a target cell that expresses a higher level of ICAM1 than CD38 on its surface. For instance, the ratio of ICAM1 to CD38 protein expression on the target cell surface is from about 1, 1.5, 2.0, 2.5, 5, 10, 15, 20, 50, 100, or 200 as measured by flow cytometry. For instance, the ratio of ICAM1 to CD38 protein expression on the target cell surface is from about 1.1 to 700, such as about 2.5, 14.2, 29.1, 64.5, 34.0, 50.3, 357.1, 666.7, based on quantification of surface expression of the proteins.

In some instances, the target cell expresses at least 500, 1000, 2000, 3000, 5000, 10000, 150000, 20000, 30000, 50000, 100000, 150000, 200000, 250000, 300000, 400000, or 500000 ICAM1 proteins on its surface as measured by flow cytometry.

In some cases, the target cell expresses at least 100, 200, 300, 400, 500, 1000, 2000, 3000, 4000, or 5000 CD38 proteins on its surface as measured by flow cytometry. In some instances, the target cell expresses less than 350,000, 300,000, 250,000, 200,000, 150,000, 100,000, 50,000, 25,000 CD38 proteins on its surface as measured by flow cytometry. In some instances the number of CD38 proteins on the surface of a target cell is 100 to 350,000, 100 to 300,000, 100 to 250,000, 100 to 200,000, 100 to 150,000, 100 to 100,000, 100 to 80,000, 100 to 60,000, 100 to 50,000, 100 to 40,000, 100 to 30,000, 100 to 20,000, 100 to 10,000, 300 to 350,000, 300 to 300,000, 300 to 250,000, 300 to 200,000, 300 to 150,000, 300 to 100,000, 300 to 80,000, 300 to 60,000, 300 to 50,000, 300 to 40,000, 300 to 30,000, 300 to 20,000, or 300 to 10,000. In some instances, the target cell has fewer CD38 proteins on its surface than a Daudi cell, a Raji cell, a KMS-26 cell, a HuNS1 cell, a HCC44 cell, or a DU145 cell. In some instances, the target cell has at least 200 CD38 proteins on its surface but fewer CD38 proteins on its surface than a Daudi cell, a Raji cell, a KMS-26 cell, a HuNS1 cell, a HCC44 cell, or a DU145 cell.

In some embodiments, the target cell is a transformed cell, wherein the ratio of ICAM1 to CD38 proteins is at least 1, 2, 5, 10, 15, 20, 35, 40, 50, or 200. In some cases, the transformed cell expresses at least 100, 200, 300, 400, 500, 750, 1000, 1250, 1500, 2000, 3000 CD38 proteins on its surface. In some cases, the target cell is a myeloma cell, a lymphoma cell, a pancreatic cancer cell, a lung adenocarcinoma cell, or a prostate carcinoma cell wherein, the ratio of ICAM1 to CD38 protein is at least 0.15, 0.20, 0.3, 0.4, 0.5, 1.0, 2.5, or 5.0. In other cases, the target cell is derived from a lung adenocarcinoma, wherein the ratio of ICAM1 to CD38 proteins is at least 1, 2, 5, 10, 15, 20, 35, 40, 50, or 200. In some cases, the myeloma cell, a lymphoma cell, a pancreatic cancer cell, a lung adenocarcinoma cell, or a prostate carcinoma cell expresses at least 100, 200, 300, 400, 500, 750, 1000, 1250, 1500, 2000, 3000 CD38 cells on its surface.

In some embodiments, a humanized bispecific antibody with a CD38-binding domain and an ICAM1-binding domain has enhanced affinity for a cell that expresses CD38 and ICAM1 compared to a monospecific protein with a CD38-binding domain and/or a monospecific protein with an ICAM1-binding domain. In some instances, the humanized bispecific antibody has 1.5, 2, 3, 4, 5, or 10-fold higher affinity for the CD38-expressing cell than a monospecific protein that binds to CD38 or a monospecific protein that binds to ICAM1. In some embodiments, a humanized bispecific antibody with a CD38-binding domain and an ICAM1-binding domain has enhanced affinity for a cell that expresses higher levels of CD38 than ICAM1 compared to a monospecific protein with a CD38-binding domain. In some instances, the humanized bispecific antibody has 1.5, 2, 3, 4, 5, or 10-fold higher affinity for the cell with higher ICAM1 expression than CD38 expression compared to a monospecific protein that binds to CD38.

In some embodiments, larger amounts of a humanized bispecific antibody with a CD38-binding domain and an ICAM1-binding domain bind to the surface of a cell that expresses CD38 and ICAM1 compared to a humanized bispecific antibody with a CD38-binding domain and/or a humanized bispecific antibody with an ICAM1-binding domain. In some instances, 1.5, 2, 3, 4, 5, or 10 -fold more of the humanized bispecific antibody binds to the CD38-expressing cell than a monospecific protein that binds to CD38 or a humanized bispecific antibody that binds to ICAM1. In some embodiments, larger amounts of a humanized bispecific antibody with a CD38-binding domain and an ICAM1-binding domain bind to the surface of a cell with higher ICAM1 expression than CD38 expression compared to a monospecific protein with a CD38-binding domain. In some instances, 1.5, 2, 3, 4, 5, or 10-fold more of the humanized bispecific antibody binds to the cell expressing more ICAM1 than CD38 compared to a monospecific protein that binds to CD38.

### 5.6 Immunological Activity of Humanized Bispecific Antibodies on Target Cells

In some embodiments, a humanized bispecific antibody with a CD38-binding domain and an ICAM1-binding domain has a higher immunologic activity against a CD38-expressing cell compared to a monospecific antibody with a CD38-binding domain and/or a monospecific antibody with an ICAM1-binding domain. In some instances, the humanized bispecific antibody has a 1.5, 2, 3, 4, 5, or 10-fold higher immunological activity than a monospecific antibody with a CD38-binding domain and/or a monospecific antibody with an ICAM1-binding domain. Various immunological activities of a humanized bispecific antibody can be measured in in vitro assays such as an ADCC assay and a CDC assay. In some instances, the humanized bispecific antibody has a 1.5, 2, 3, 4, 5, or 10-fold higher ADCC activity than a monospecific antibody with a CD38-binding domain and/or a monospecific antibody with an ICAM1-binding domain. In some instances, the humanized bispecific antibody has a 1.5, 2, 3, 4, 5, or 10-fold higher CDC activity than a humanized monospecific antibody with a CD38-binding domain and/or a monospecific antibody with an ICAM1-binding domain.

In some instances, the humanized bispecific antibody further comprises an enhanced CDC effect compared to a CDC effect by reference antibody daratumumab. In some instances, the humanized bispecific antibody further comprises an enhanced ADCC effect compared to an ADCC effect by reference antibody daratumumab. In some instances, the humanized bispecific antibody further comprises a reduced immune cell killing effect compared to an immune cell killing effect of reference antibody daratumumab. In some cases, the enhanced CDC is at least 2-fold, 3-fold, 4-fold, or higher than the CDC effect of reference antibody daratumumab. In some cases, the enhanced CDC is at least 30%, 40%, 50%, 60%, 70%, 80%, 90%, or higher than the CDC effect of reference antibody daratumumab. In some cases, the enhanced ADCC is at least 2-fold, 3-fold, 4-fold, 5-fold, or higher than the ADCC effect of reference antibody daratumumab. In some cases, the enhanced ADCC is at least 30%, 40%, 50%, 60%, 70%, 80%, 90%, or higher than the ADCC effect of reference antibody daratumumab. In some cases, the immune cell is a Natural Killer cell. In some cases, the immune cell viability is improved by about 10%, 20%, 30%, 40%, 50%, 60%, 70%, 80%, 90%, or higher compared to the immune cell viability in the presence of reference antibody daratumumab.

In some embodiments, described herein is a humanized bispecific antibody which comprises a first targeting moiety that specifically binds to CD38 and a second targeting moiety that specifically binds to ICAM1. In some instances, the humanized bispecific antibody further comprises an enhanced CDC effect compared to a CDC effect by reference antibody daratumumab. In some instances, the humanized bispecific antibody further comprises an enhanced ADCC effect compared to an ADCC effect by reference antibody daratumumab. In some cases, the enhanced CDC is at least 2-fold, 3-fold, 4-fold, or higher than the CDC effect of reference antibody daratumumab. In some cases, the enhanced CDC is at least 30%, 40%, 50%, 60%, 70%, 80%, 90%, or higher than the CDC effect of reference antibody daratumumab. In some cases, the enhanced ADCC is at least 2-fold, 3-fold, 4-fold, 5-fold, or higher than the ADCC effect of reference antibody daratumumab. In some cases, the enhanced ADCC is at least 30%, 40%, 50%, 60%, 70%, 80%, 90%, or higher than the ADCC effect of reference antibody daratumumab.

In some embodiments, described herein is a humanized bispecific antibody comprising a first component that binds specifically to CD38 and a second component that binds specifically to ICAM1, wherein the humanized bispecific antibody mediates ADCC more efficiently than a monospecific antibody that comprises either the first component or the second component, wherein the ADCC activity is determined using an in vitro cytotoxicity assay. In some embodiments, the humanized bispecific antibody mediates at least about 5%, at least about 10%, at least about 20%, at least about 30%, at least about 40%, at least about 50%, at least about 75%, at least about 100% higher maximum cytotoxicity in an in vitro ADCC assay than the monospecific antibody that comprises either the first component or the second component. In some embodiments, the humanized bispecific antibody mediates at least 2-fold, at least 3-fold, at least 4-fold, at least 5-fold, or at least 10-fold higher maximum cytotoxicity in an in vitro ADCC assay than the monospecific antibody that comprises either the first component or the second component.

In some embodiments, described herein is a humanized bispecific antibody comprising a first component that binds specifically to CD38 and a second component that binds specifically to ICAM1, wherein the humanized bispecific antibody mediates CDC more efficiently than a monospecific antibody that comprises either the first component or the second component, wherein the ADCC activity is determined using an in vitro cytotoxicity assay, wherein the CDC activity is determined using an in vitro cytotoxicity assay. In some embodiments, the humanized bispecific antibody mediates at least about 5%, at least about 10%, at least about 20%, at least about 30%, at least about 40%, at least about 50%, at least about 75%, at least about 100% higher maximum cytotoxicity in an in vitro CDC assay than the monospecific antibody that comprises either the first component or the second component.

In some instances, the humanized bispecific antibody further comprises a reduced immune cell killing effect compared to an immune cell killing effect of reference antibody daratumumab. In some cases, the immune cell viability is improved by about 10%, 20%, 30%, 40%, 50%, 60%, 70%, 80%, 90%, or higher compared to the immune cell viability in the presence of reference antibody daratumumab. In some cases, the immune cell is a Natural Killer cell.

Immunological activity can also be measured in a cell-line derived xenograft assay, wherein transformed cells are injected into mice and form a tumor. In some instances, a humanized bispecific antibody with a CD38-binding domain and an ICAM1-binding domain inhibits the growth of a tumor comprising CD38-expressing cells to a greater extent than a humanized bispecific antibody with a CD38-binding domain and/or a monospecific protein with an ICAM1-binding domain. In some instances, the humanized bispecific antibody exhibits 1.5, 2, 3, 4, 5, or 10-fold higher inhibition of xenograft tumor growth compared to a monospecific protein with a CD38-binding domain and/or a monospecific protein with an ICAM1-binding domain.

As used herein, “Antibody-dependent cell-mediated cytotoxicity” and “ADCC” refer to a cell-mediated reaction in which non-specific cytotoxic cells (e.g., Natural Killer (NK) cells, neutrophils, and macrophages) recognize bound antibody on a target cell and subsequently cause lysis of the target cell. In one embodiment, the target cell is a human cell, such as a tumor cell (e.g., a myeloma cell). While not wishing to be bound by any particular mechanism of action, the cytotoxic cells that mediate ADCC generally express Fc receptors (FcRs). Cells for mediating ADCC, NK cells, express FcγRIII, whereas monocytes express FcγRI, FcγRII, FcγRIII and/or FcγRIV. FcR expression on hematopoietic cells is summarized in Ravetch and Kinet, Annu. Rev. Immunol., 9:457-92 (1991).

To assess ADCC activity of a humanized bispecific antibody as described herein, an in vitro ADCC assay, such as a cytotoxic assay using a cancer cell lines, is carried out, in some embodiments. Useful effector cells for such assays include, but are not limited to, peripheral blood mononuclear cells (PBMC) and Natural Killer (NK) cells. Alternatively, or additionally, ADCC activity of the humanized bispecific antibody of interest is assessed, in some embodiments, in vivo, e.g., in an animal.

“Complement-dependent cytotoxicity” or “CDC” refers to the ability of a molecule to initiate complement activation leading to lysis of a target cell in the presence of complement. The complement activation pathway is initiated by the binding of the first component of the complement system (C1q) to a molecule (e.g., an antibody) complexed with a cognate antigen. To assess complement activation, a CDC assay, e.g., as described in Gazzano-Santaro et al., J. Immunol. Methods, 202:163 (1996), is performed, in some embodiments.

In some embodiments, a humanized bispecific antibody as described herein, that binds CD38 and ICAM1 mediates complement-dependent lysis of at least 50% of the cells in an exponentially growing population of Raji cells, at a concentration of about 2 nM. In some embodiments, a multispecific protein as described herein that binds CD38 and ICAM1 mediates ADCC by PBMC cells of 40% of the cells in an exponentially growing population of Daudi cells, at a concentration of about 1 nM. In certain embodiments, a humanized bispecific antibody as described herein, that binds CD38 and ICAM1, does not induce apoptosis without crosslinking.

### 5.7 Production of Antibodies or Binding Fragments Thereof

In some embodiments, polypeptides described herein (e.g., antibodies and its binding fragments) are produced using any method known in the art to be useful for the synthesis of polypeptides (e.g., antibodies), in particular, by chemical synthesis or by recombinant expression, and are preferably produced by recombinant expression techniques.

In some instances, an antibody or its binding fragment thereof is expressed recombinantly, and the nucleic acid encoding the antibody or its binding fragment is assembled from chemically synthesized oligonucleotides (e.g., as described in Kutmeier et al., 1994, BioTechniques 17:242), which involves the synthesis of overlapping oligonucleotides containing portions of the sequence encoding the antibody, annealing and ligation of those oligonucleotides, and then amplification of the ligated oligonucleotides by PCR.

Alternatively, a nucleic acid molecule encoding an antibody is optionally generated from a suitable source (e.g., an antibody cDNA library, or a cDNA library generated from any tissue or cells expressing the immunoglobulin) by PCR amplification using synthetic primers hybridizable to the 3′ and 5′ ends of the sequence or by cloning using an oligonucleotide probe specific for the particular gene sequence.

In some instances, an antibody or its binding is optionally generated by immunizing an animal, such as a rabbit, to generate polyclonal antibodies or, more preferably, by generating monoclonal antibodies, e.g., as described by Kohler and Milstein (1975, Nature 256:495-497) or, as described by Kozbor et al. (1983, Immunology Today 4:72) or Cole et al. (1985 in Monoclonal Antibodies and Cancer Therapy, Alan R. Liss, Inc., pp. 77-96). Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

In some embodiments, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used. A chimeric antibody is a molecule in which different portions are derived from different animal species, such as those having a variable region derived from a murine monoclonal antibody and a human immunoglobulin constant region.

In some embodiments, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988, Science 242:1038-1041).

In some embodiments, an expression vector comprising the nucleotide sequence of an antibody or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In specific embodiments, the expression of the antibody is regulated by a constitutive, an inducible or a tissue, specific promoter.

In some embodiments, a variety of host-expression vector systems is utilized to express an antibody, or its binding fragment described herein. Such host-expression systems represent vehicles by which the coding sequences of the antibody is produced and subsequently purified, but also represent cells that are, when transformed or transfected with the appropriate nucleotide coding sequences, express an antibody or its binding fragment in situ. These include, but are not limited to, microorganisms such as bacteria (e.g., E. coli and B. subtilis) transformed with recombinant bacteriophage DNA, plasmid DNA or cosmid DNA expression vectors containing an antibody or its binding fragment coding sequences; yeast (e.g., Saccharomyces Pichia) transformed with recombinant yeast expression vectors containing an antibody or its binding fragment coding sequences; insect cell systems infected with recombinant virus expression vectors (e.g., baculovirus) containing an antibody or its binding fragment coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing an antibody or its binding fragment coding sequences; or mammalian cell systems (e.g., COS, CHO, BH, 293, 293T, 3T3 cells) harboring recombinant expression constructs containing promoters derived from the genome of mammalian cells (e.g., metallothionein promoter) or from mammalian viruses (e.g. the adenovirus late promoter; the vaccinia virus 7.5 K promoter).

For long-term and high-yield production of recombinant proteins, stable expression is preferred. In some instances, cell lines that stably express an antibody are optionally engineered. Rather than using expression vectors that contain viral origins of replication, host cells are transformed with DNA controlled by appropriate expression control elements (e.g., promoter, enhancer, sequences, transcription terminators, polyadenylation sites, etc.), and a selectable marker. Following the introduction of the foreign DNA, engineered cells are then allowed to grow for 1-2 days in an enriched media and then are switched to a selective media. The selectable marker in the recombinant plasmid confers resistance to the selection and allows cells to stably integrate the plasmid into their chromosomes and grow to form foci that in turn are cloned and expanded into cell lines. This method can advantageously be used to engineer cell lines which express the antibody or its binding fragments.

In some instances, a number of selection systems are used, including but not limited to the herpes simplex virus thymidine kinase (Wigler et al., 1977, Cell 11:223), hypoxanthine-guanine phosphoribosyltransferase (Szybalska & Szybalski, 192, Proc. Natl. Acad. Sci. USA 48:202), and adenine phosphoribosyltransferase (Lowy et al., 1980, Cell 22:817) genes are employed in tk-, hgprt- or aprt- cells, respectively. Also, antimetabolite resistance are used as the basis of selection for the following genes: dhfr, which confers resistance to methotrexate (Wigler et al., 1980, Proc. Natl. Acad. Sci. USA 77:357; O’Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May 1993, TIB TECH 11(5):155-215) and hygro, which confers resistance to hygromycin (Santerre et al., 1984, Gene 30:147). Methods commonly known in the art of recombinant DNA technology which can be used are described in Ausubel et al. (eds., 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J. Mol. Biol. 150:1).

In some instances, the expression levels of an antibody are increased by vector amplification (for a review, see Bebbington and Hentschel, the use of vectors based on gene amplification for the expression of cloned genes in mammalian cells in DNA cloning, Vol. 3. (Academic Press, New York, 1987)). When a marker in the vector system expressing an antibody is amplifiable, an increase in the level of inhibitor present in the culture of host cell will increase the number of copies of the marker gene. Since the amplified region is associated with the nucleotide sequence of the antibody, production of the antibody will also increase (Crouse et al., 1983, Mol. Cell Biol. 3:257).

In some instances, any method known in the art for purification of an antibody is used, for example, by chromatography (e.g., ion exchange, affinity, particularly by affinity for the specific antigen after Protein A, and sizing column chromatography), centrifugation, differential solubility, or by any other standard technique for the purification of proteins.

### 5.8 Expression Vectors

In some embodiments, vectors include any suitable vectors derived from eukaryotic or prokaryotic sources. In some cases, vectors are obtained from bacteria (e.g. E. coli), insects, yeast (e.g. Pichia pastoris), algae, or mammalian sources. Exemplary bacterial vectors include pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Exemplary insect vectors include pFastBac1, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

In some cases, yeast vectors include Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichi pastoris vector, pGAPZA,B, & C Pichia pastoris vector, pPIC3.5 K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Exemplary algae vectors include pChlamy-4 vector or MCS vector.

Examples of mammalian vectors include transient expression vectors or stable expression vectors. Mammalian transient expression vectors may include pRK5, p3xFLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3xFLAG-CMV 7.1, pFLAG-CMV 20, p3xFLAG-Myc-CMV 24, pCMV-FLAG-MAT1, pCMV-FLAG-MAT2, pBICEP-CMV 3, or pBICEP-CMV 4. Mammalian stable expression vector may include pFLAG-CMV 3, p3xFLAG-CMV 9, p3xFLAG-CMV 13, pFLAG-Myc-CMV 21, p3xFLAG-Myc-CMV 25, pFLAG-CMV 4, p3xFLAG-CMV 10, p3xFLAG-CMV 14, pFLAG-Myc-CMV 22, p3xFLAG-Myc-CMV 26, pBICEP-CMV 1, or pBICEP-CMV 2.

In some instances, a cell-free system is a mixture of cytoplasmic and/or nuclear components from a cell and is used for in vitro nucleic acid synthesis. In some cases, a cell-free system utilizes either prokaryotic cell components or eukaryotic cell components. Sometimes, a nucleic acid synthesis is obtained in a cell-free system based on for example Drosophila cell, Xenopus egg, or HeLa cells. Exemplary cell-free systems include, but are not limited to, E. coli S30 Extract system, E. coli T7 S30 system, or PURExpress®.

### 5.9 Host Cells

In some embodiments, a host cell includes any suitable cell such as a naturally derived cell or a genetically modified cell. In some instances, a host cell is a production host cell. In some instances, a host cell is a eukaryotic cell. In other instances, a host cell is a prokaryotic cell. In some cases, a eukaryotic cell includes fungi (e.g., yeast cells), animal cell or plant cell. In some cases, a prokaryotic cell is a bacterial cell. Examples of bacterial cell include gram-positive bacteria or gram-negative bacteria. Sometimes the gram-negative bacteria is anaerobic, rod-shaped, or both.

In some instances, gram-positive bacteria include Actinobacteria, Firmicutes, or Tenericutes. In some cases, gram-negative bacteria include Aquificae, Deinococcus-Thermus, Fibrobacteres-Chlorobi/Bacteroidetes (FCB group), Fusobacteria, Gemmatimonadetes, Nitrospirae, Planctomycetes-Verrucomicrobia/ Chlamydiae (PVC group), Proteobacteria, Spirochaetes, or Synergistetes. Other bacteria can be Acidobacteria, Chloroflexi, Chrysiogenetes, Cyanobacteria, Deferribacteres, Dictyoglomi, Thermodesulfobacteria, or Thermotogae. A bacterial cell can be Escherichia coli, Clostridium botulinum, or Coli bacilli.

Exemplary prokaryotic host cells include, but are not limited to, BL21, Mach1™, DH10B™, TOP10, DH5α, DH10Bac™, OmniMax™, MegaX™, DH12S™, INV110, TOP10F′, INVαF, TOP10/P3, ccdB Survival, PIR1, PIR2, Stbl2™, Stbl3™, or Stbl4™.

In some instances, an animal cell includes a cell from a vertebrate or from an invertebrate. In some cases, an animal cell includes a cell from a marine invertebrate, fish, insects, amphibian, reptile, or mammal. In some cases, a fungus cell includes a yeast cell, such as brewer’s yeast, baker’s yeast, or wine yeast.

Fungi include ascomycetes such as yeast, mold, filamentous fungi, basidiomycetes, or zygomycetes. In some instances, yeast includes Ascomycota or Basidiomycota. In some cases, Ascomycota includes Saccharomycotina (true yeasts, e.g. Saccharomyces cerevisiae (baker’s yeast)) or Taphrinomycotina (e.g. Schizosaccharomycetes (fission yeasts)). In some cases, Basidiomycota includes Agaricomycotina (e.g. Tremellomycetes) or Pucciniomycotina (e.g. Microbotryomycetes).

Exemplary yeast or filamentous fungi include, for example, the genus: Saccharomyces, Schizosaccharomyces, Candida, Pichia, Hansenula, Kluyveromyces, Zygosaccharomyces, Yarrowia, Trichosporon, Rhodosporidi, Aspergillus, Fusarium, or Trichoderma. Exemplary yeast or filamentous fungi include, for example, the species: Saccharomyces cerevisiae, Schizosaccharomyces pombe, Candida utilis, Candida boidini, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Pichia metanolica, Pichia angusta, Pichia pastoris, Pichia anomala, Hansenula polymorpha, Kluyveromyces lactis, Zygosaccharomyces rouxii, Yarrowia lipolytica, Trichosporon pullulans, Rhodosporidium toru-Aspergillus niger, Aspergillus nidulans, Aspergillus awamori, Aspergillus oryzae, Trichoderma reesei, Yarrowia lipolytica, Brettanomyces bruxellensis, Candida stellata, Schizosaccharomyces pombe, Torulaspora delbrueckii, Zygosaccharomyces bailii, Cryptococcus neoformans, Cryptococcus gattii, or Saccharomyces boulardii.

Exemplary yeast host cells include, but are not limited to, Pichia pastoris yeast strains such as GS115, KM71H, SMD1168, SMD1168H, and X-33; and Saccharomyces cerevisiae yeast strain such as INVSc1.

In some instances, additional animal cells include cells obtained from a mollusk, arthropod, annelid, or sponge. In some cases, an additional animal cell is a mammalian cell, e.g., from a primate, ape, equine, bovine, porcine, canine, feline or rodent. In some cases, a rodent includes mouse, rat, hamster, gerbil, hamster, chinchilla, fancy rat, or guinea pig.

Exemplary mammalian host cells include, but are not limited to, 293A cell line, 293FT cell line, 293F cells, 293 H cells, CHO DG44 cells, CHO-S cells, CHO-K1 cells, FUT8 KO CHO-K1, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, and T-REx™-HeLa cell line.

In some instances, a mammalian host cell is a stable cell line or a cell line that has incorporated a genetic material of interest into its own genome and has the capability to express the product of the genetic material after many generations of cell division. In some cases, a mammalian host cell is a transient cell line” or a cell line that has not incorporated a genetic material of interest into its own genome and does not have the capability to express the product of the genetic material after many generations of cell division.

Exemplary insect host cells include, but are not limited to, Drosophila S2 cells, Sf9 cells, Sf21 cells, High Five™ cells, and expresSF+® cells.

In some instances, plant cells include a cell from algae. Exemplary algal cell lines include, but are not limited to, strains from Chlamydomonas reinhardtii 137c, or Synechococcus elongatus PPC 7942.

### 5.10 Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an,” and “the” include plural referents unless the context clearly dictates otherwise. In this application, the use of “or” means “and/or” unless stated otherwise. Furthermore, use of the term “including” as well as other forms, such as “include,” “includes,” and “included,” is not limiting.

As used herein, ranges and amounts can be expressed as “about” a particular value or range. About also includes the exact amount. Hence “about 5 µL” means “about 5 µL” and also “5 µL.” Generally, the term “about” includes an amount that would be expected to be within experimental error.

The section headings used herein are for organizational purposes only and are not to be construed as limiting the subject matter described.

“Antibodies” and “immunoglobulins” (IGs) are glycoproteins having the same structural characteristics. The terms are used synonymously. In some instances, the antigen specificity of the immunoglobulin is known.

The term “antibody” is used in the broadest sense, and covers fully assembled antibodies, antibody fragments that can bind antigen (e.g., Fab, F(ab′)2, Fv, single chain antibodies (scFv), diabodies, antibody chimeras, hybrid antibodies, bispecific antibodies, and the like), and recombinant peptides comprising the forgoing.

The terms “monoclonal antibody” and “mAb” as used herein refer to an antibody obtained from a substantially homogeneous population of antibodies, i.e., the individual antibodies comprising the population are identical except for possible naturally occurring mutations that may be present in minor amounts.

Native antibodies” and “native immunoglobulins” are usually heterotetrameric glycoproteins of about 150,000 Daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy-chain variable domains.

The term “variable” refers to the fact that certain portions of the variable domains differ extensively in sequence among antibodies. Variable regions confer antigen-binding specificity. However, the variability is not evenly distributed throughout the variable domains of antibodies. It is concentrated in three segments called complementarity-determining regions (CDRs) or hypervariable regions, both in the light chain and the heavy-chain variable domains. The more highly conserved portions of variable domains are referred to as framework (FR) regions. The variable domains of native heavy and light chains each comprise four FR regions, largely adopting a β-pleated-sheet configuration, connected by three CDRs, which form loops connecting, and in some cases forming part of, the β-pleated-sheet structure. The CDRs in each chain are held together in close proximity by the FR regions and, with the CDRs from the other chain, contribute to the formation of the antigen-binding site of antibodies (see, Kabat et al. (1991) NIH PubL. No. 91-3242, Vol. I, pages 647-669). The constant domains are not involved directly in binding an antibody to an antigen, but exhibit various effector functions, such as Fc receptor (FcR) binding, participation of the antibody in antibody-dependent cellular toxicity, initiation of CDC, and mast cell degranulation.

The term “hypervariable region,” when used herein, refers to the amino acid residues of an antibody that are responsible for antigen-binding. The hypervariable region comprises amino acid residues from a “complementarity-determining region” or “CDR” (i.e., residues 24-34 (L1), 50-56 (L2), and 89-97 (L3) in the light-chain variable domain and 31-35 (H1), 50-65 (H2), and 95-102 (H3) in the heavy-chain variable domain; Kabat et al. (1991) Sequences of Proteins of Immunological Interest, 5th Ed. Public Health Service, National Institute of Health, Bethesda, Md.) and/or those residues from a “hypervariable loop” (i.e., residues 26-32 (L1), 50-52 (L2), and 91-96 (L3) in the light-chain variable domain and (H1), 53-55 (H2), and 96-101 (13) in the heavy chain variable domain; Clothia and Lesk, (1987) J. Mol. Biol., 196:901-917). “Framework” or “FR” residues are those variable domain residues other than the hypervariable region residues, as herein deemed.

“Antibody fragments” comprise a portion of an intact antibody, preferably the antigen-binding or variable region of the intact antibody. Examples of antibody fragments include Fab, Fab, F(ab′)2, and Fv fragments; diabodies; linear antibodies (Zapata et al. (1995) Protein Eng. 10:1057-1062); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

“Fv” is the minimum antibody fragment that contains a complete antigen recognition and binding site. This region consists of a dimer of one heavy- and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

“scFv” is a single chain antibody fragment that contains three CDRs from a heavy chain variable domain and three CDRs from a light chain in a single polypeptide. The three CDRs derived from each variable domain interact to define an antigen-binding site on the surface of the scFv polypeptide.

The Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. Fab′ fragments are produced by reducing the F(ab′)2 fragment’s heavy chain disulfide bridge. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, immunoglobulins can be assigned to different classes. There are five major classes of human immunoglobulins: IgA, IgD, IgE, IgG, IgM, and IgY, and several of these may be further divided into subclasses (isotypes), e.g., IgG1, IgG2, IgG3, IgG4, IgA1, and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. Different isotypes have different effector functions. For example, human IgG1 and IgG3 isotypes have ADCC (antibody-dependent cell-mediated cytotoxicity) activity.

In some instances, the CDRs of an antibody are determined according to (i) the Kabat numbering system (Kabat et al. (197 ) Ann. NY Acad. Sci. 190:382-391 and, Kabat et al. (1991) Sequences of Proteins of Immunological Interest Fifth Edition, U.S. Department of Health and Human Services, NIH Publication No. 91-3242); or (ii) the Chothia numbering scheme, which will be referred to herein as the “Chothia CDRs” (see, e.g., Chothia and Lesk, 1987, J. Mol. Biol., 196:901-917; Al-Lazikani et al., 1997, J. Mol. Biol., 273 :927-948; Chothia et al., 1992, J. Mol. Biol., 227:799-817; Tramontano A et al., 1990, J. Mol. Biol. 215(1): 175-82; and U.S. Pat. No. 7,709,226); or (iii) the ImMunoGeneTics (IMGT) numbering system, for example, as described in Lefranc, M.-P., 1999, The Immunologist, 7: 132-136 and Lefranc, M.-P. et al, 1999, Nucleic Acids Res., 27:209-212 (“IMGT CDRs”); or (iv) MacCallum et al, 1996, J. Mol. Biol., 262:732-745. See also, e.g., Martin, A., “Protein Sequence and Structure Analysis of Antibody Variable Domains,” in Antibody Engineering, Kontermann and Diibel, eds., Chapter 31, pp. 422-439, Springer- Verlag, Berlin (2001).

With respect to the Kabat numbering system, CDRs within an antibody heavy chain molecule are typically present at amino acid positions 31 to 35, which optionally can include one or two additional amino acids, following 35 (referred to in the Kabat numbering scheme as 35 A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). As is well known to those of skill in the art, using the Kabat numbering system, the actual linear amino acid sequence of the antibody variable domain can contain fewer or additional amino acids due to a shortening or lengthening of a FR and/or CDR and, as such, an amino acid’s Kabat number is not necessarily the same as its linear amino acid number.

The term “chimeric” antibody refers to an antibody in which a portion of the heavy and/or light chain is derived from a particular source or species, while the remainder of the heavy and/or light chain is derived from a different source or species.

The term “human antibody” or “humanized antibody,” as used herein, is intended to include antibodies having variable and constant regions derived from human germ-line immunoglobulin sequences. Human antibodies are well-known in the state of the art (van Dijk, M.A., and van de Winkel, J.G., Curr. Opin. Chem. Biol. 5 (2001) 368-374). In some instances, human antibodies are also produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al, Proc. Natl. Acad. Sci. USA 90 (1993) 2551-2555; Jakobovits, A., et al, Nature 362 (1993) 255-258; Bruggemann, M., et al, Year Immunol. 7 (1993) 33-40). In additional instances, human antibodies are also produced in phage display libraries (Hoogenboom, H.R., and Winter, G., J. Mol. Biol. 227 (1992) 381-388; Marks, J.D., et al, J. Mol. Biol. 222 (1991) 581-597). The techniques of Cole et al. and Boerner et al. are also available for the preparation of human monoclonal antibodies (Cole, et al., Monoclonal Antibodies and Cancer Therapy, Alan R. Liss, p. 77 (1985); and Boerner, P., et al, J. Immunol. 147 (1991) 86-95).

The term “recombinant human antibody,” as used herein, is intended to include all human antibodies that are prepared, expressed, created or isolated by recombinant means, such as antibodies isolated from a host cell such as a NSO or CHO cell or from an animal (e.g. a mouse) that is transgenic for human immunoglobulin genes or antibodies expressed using a recombinant expression vector transfected into a host cell. Such recombinant human antibodies have variable and constant regions in a rearranged form. In some cases, the recombinant human antibodies have been subjected to in vivo somatic hypermutation. Thus, the amino acid sequences of the VH and VL regions of the recombinant antibodies are sequences that, while derived from and related to human germ-line VH and VL sequences, may not naturally exist within the human antibody germ-line repertoire in vivo.

As used herein, the terms “individual(s),” “subject(s),” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician’s assistant, an orderly or a hospice worker).

As used herein, the term “anti-CD38 BMK” antibody refers to reference antibody daratumumab.

As used herein, the term “Percent (%) amino acid sequence identity” with respect to a sequence is defined as the percentage of amino acid residues in a candidate sequence that are identical with the amino acid residues in the specific sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as EMBOSS MATCHER, EMBOSS WATER, EMBOSS STRETCHER, EMBOSS NEEDLE, EMBOSS LALIGN, BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

As used herein, the term “afucosylated” refers to a glycosylated polypeptide with reduced fucose residues compared to a glycosylated polypeptide expressed in a wild-type cell line or a healthy individual. For example, glycosylated polypeptides expressed in a CHO FUT8 -/- mutant cell line are afucosylated. An afucosylated glycosylated polypeptide is not required to have zero fucose residues.

### 6. LIST OF EMBODIMENTS

The following list of embodiments of the invention are to be considered as disclosing various features of the invention, which features can be considered to be specific to the particular embodiment under which they are discussed, or which are combinable with the various other features as listed in other embodiments. Thus, simply because a feature is discussed under one particular embodiment does not necessarily limit the use of that feature to that embodiment.

Embodiment 1. A bispecific antibody comprising one or more humanized CD38 binding domains, one or more humanized ICAM1 binding domains, and a human Fc domain.

Embodiment 2. The bispecific antibody of Embodiment 1, wherein the humanized CD38 binding domain comprises an scFv.

Embodiment 3. The bispecific antibody of Embodiment 1 or 2, wherein the humanized CD38 binding domain comprises a variable domain of an IgG heavy chain and a variable domain of an IgG light chain.

Embodiment 4. The bispecific antibody of any one of Embodiments 1-3, wherein the humanized CD38 binding domain comprises a heavy chain variable region and a light chain variable region.

Embodiment 5. The bispecific antibody of any one of Embodiments 1-4, wherein the humanized ICAM1 binding domain comprises an scFv.

Embodiment 6. The bispecific antibody of any one of Embodiments 1-5, wherein the humanized ICAM1 binding domain comprises a variable domain of an IgG heavy chain and a variable domain of an IgG light chain.

Embodiment 7. The bispecific antibody of any one of Embodiments 1-6, wherein the humanized ICAM1 binding domain comprises a heavy chain variable region and a light chain variable region.

Embodiment 8. The bispecific antibody of any one of Embodiments 1-7, wherein an isotype of the bispecific antibody is IgG1.

Embodiment 9. The bispecific antibody of any one of Embodiments 1-8, wherein the human Fc domain is a heterodimeric Fc domain, wherein the heterodimeric Fc region comprises a knob chain and a hole chain, forming a knob-in-hole (KiH) structure.

Embodiment 10. The bispecific antibody of Embodiment 9, wherein the knob chain comprises mutation T366W and the hole chain comprises mutations T366S, L368A, and Y407V, wherein amino acid position numbering is according to the EU index of Kabat et al.

Embodiment 11. The bispecific antibody of Embodiment 9, wherein the knob chain comprises mutations S354C, T366W and the hole chain comprises mutations Y349C, T366S, L368A, and Y407V, wherein amino acid position numbering is according to the EU index of Kabat et al.

Embodiment 12. The bispecific antibody of any one of Embodiments 1-11, wherein the Fc domain is afucosylated.

Embodiment 13. The bispecific antibody of any one of Embodiments 1-12, wherein the Fc domain comprises one or more amino acid substitutions that enhance antigen-dependent cellular cytotoxicity (ADCC) activity.

Embodiment 14. The bispecific antibody of Embodiment 13, wherein the Fc domain comprises S239D, I332E, and A330L amino acid substitutions, and wherein the amino acid numbering is according to the EU index in Kabat et al.

Embodiment 15. The bispecific antibody of any one of Embodiments 1-12, wherein the humanized CD38 binding domain comprises the variable domain of an anti-CD38 IgG, and the ICAM1 binding domain comprises an anti-ICAM1 single chain variable fragment (anti-ICAM1 scFv).

Embodiment 16. The bispecific antibody of any one of Embodiments 1-13, wherein the humanized ICAM1 binding domain comprises the variable domain of an anti-ICAM1 IgG, and the CD38 binding domain comprises an anti-CD38 single chain variable fragment (anti-CD38 scFv).

Embodiment 17. The bispecific antibody of any one of Embodiments 1-16, further comprising a CH1 IgG domain and a CL IgG domain.

Embodiment 18. The bispecific antibody of Embodiments 1-17, further comprising a T cell receptor (TCR) constant region, wherein the TCR constant region comprises a TCR alpha constant domain and a TCR beta constant domain.

Embodiment 19. The bispecific antibody of Embodiment 18, wherein
the humanized CD38 binding domain comprises a VH-CD38 domain and a VL-CD38 domain;
the VH-CD38 domain is fused to the TCR beta constant domain; and
the VL-CD38 domain is fused to the TCR alpha constant domain.

Embodiment 20. The bispecific antibody of Embodiment 18, wherein
the humanized CD38 binding domain comprises a VH-CD38 domain and a VL-CD38 domain;
the VH-CD38 domain is fused to the CH1 IgG domain; and
the VL-CD38 domain is fused to the CL IgG domain.

Embodiment 21. The bispecific antibody of Embodiment 18 or 20, wherein
the humanized ICAM1 binding domain comprises a VH-ICAM1 domain and a VL-ICAM1 domain;
the VH-ICAM1 domain is fused to the TCR beta constant domain; and
the VL-ICAM1 domain is fused to the TCR alpha constant domain.

Embodiment 22. The bispecific antibody of Embodiment 18 or 19, wherein
the humanized ICAM1 binding domain comprises a VH-ICAM1 domain and a VL-ICAM1 domain;
the VH-ICAM1 domain is fused to the CH1 IgG domain; and
the VL-ICAM1 domain is fused to the CL IgG domain.

Embodiment 23. The bispecific antibody of any one of Embodiments 1-22, wherein the lowest melting transition of the bispecific antibody is at least 55° C., at least 60° C. or at least 65° C.

Embodiment 24. The bispecific antibody of any one of Embodiments 1-23, wherein the humanized CD38 binding domain comprises a sequence at least 90% identical to SEQ ID NO: 40.

Embodiment 25. The bispecific antibody of any one of Embodiments 1-24, wherein the humanized CD38 binding domain comprises a sequence at least 90% identical to SEQ ID NO: 41.

Embodiment 26. The bispecific antibody of any one of Embodiments 1-24, wherein the humanized CD38 binding domain comprises a sequence at least 90% identical to SEQ ID NO: 35.

Embodiment 27. The bispecific antibody of any one of Embodiments 1-25, wherein the humanized CD38 binding domain comprises sequences at least 90% identical to SEQ ID NO: 50, SEQ ID NO: 51, SEQ ID NO: 52, SEQ ID NO: 53, SEQ ID NO: 54, and SEQ ID NO: 55.

Embodiment 28. The bispecific antibody of Embodiment 27, wherein the HC CDR3 domain comprises glutamic acid 95, glutamic acid 100b, glycine 100c, and tyrosine 100d, and the LC CDR3 domain comprises glycine 90, tyrosine 91, serine 93, glycine 94, and tyrosine 96, wherein amino acid position numbering is according to the EU index of Kabat et al.

Embodiment 29. The bispecific antibody of any one of Embodiments 1-28, wherein the humanized ICAM1 binding domain comprises a sequence at least 90% identical to SEQ ID NO: 44.

Embodiment 30. The bispecific antibody of any one of Embodiments 1-29, wherein the humanized ICAM1 binding domain comprises a sequence at least 90% identical to SEQ ID NO: 45.

Embodiment 31. The bispecific antibody of any one of Embodiments 1-30, wherein the humanized ICAM1 binding domain comprises sequences at least 90% identical to SEQ ID NO: 56, SEQ ID NO: 57, SEQ ID NO: 58, SEQ ID NO: 59, SEQ ID NO: 60, and SEQ ID NO: 61.

Embodiment 32. The bispecific antibody of any one of Embodiments 1-31, wherein the humanized CD38 binding domain is capable of binding to the extracellular domain of human CD38 with an equilibrium dissociation constant (KD) of less than 10 nM or less than 5 nM.

Embodiment 33. The bispecific antibody of any one of Embodiments 1-31, wherein the humanized CD38 binding domain is capable of binding to the extracellular domain of human CD38 with an equilibrium dissociation constant (KD) of between 0.1 nM and 20 nM, between 0.5 nM and 15 nM, between 1 nM and 10 nM, or between 1 nM and 5 nM.

Embodiment 34. The bispecific antibody of any one of Embodiments 1-31, wherein the humanized CD38 binding domain is capable of binding to the extracellular domain of human CD38 with an equilibrium dissociation constant (KD) of between 2 nM and 5 nM.

Embodiment 35. The bispecific antibody of any one of Embodiments 1-34, wherein the humanized ICAM1 binding domain is capable of binding to the extracellular domain of human ICAM1 with an equilibrium dissociation constant (KD) of less than 1 nM, less than 0.5 nM, or less than 0.2 nM.

Embodiment 36. The bispecific antibody of any one of Embodiments 1-34, wherein the humanized ICAM1 binding domain is capable of binding to the extracellular domain of human ICAM1 with an equilibrium dissociation constant (KD) of between 0.02 nM and 10 nM, between 0.05 nM and 5 nM, between 0.05 nM and 1 nM, or between 0.1 nM and 0.5 nM.

Embodiment 37. The bispecific antibody of any one of Embodiments 1-34, wherein the humanized ICAM1 binding domain is capable of binding to the extracellular domain of human ICAM1 with an equilibrium dissociation constant (KD) of between 0.1 nM and 0.15 nM.

Embodiment 38. The bispecific antibody of any one of Embodiments 32-37, wherein the KD is determined by surface plasmon resonance.

Embodiment 39. The bispecific antibody of any of Embodiments 1-38, wherein the bispecific antibody comprises an afucosylated Fc domain.

Embodiment 40. The bispecific antibody of Embodiment 39, wherein the bispecific antibody is capable of inducing an enhanced ADCC effect on a target cell compared to an ADCC effect induced on the target cell by a an otherwise identical bispecific antibody that does not comprise an afucosylated Fc domain.

Embodiment 41. The bispecific antibody of any one of Embodiments 1-40, wherein the bispecific antibody is capable of inducing an enhanced ADCC effect on a target cell compared to an ADCC effect induced on the target cell by a monospecific protein that comprises the humanized CD38 binding domain or the humanized ICAM1 binding domain.

Embodiment 42. The bispecific antibody of any one of Embodiments 1-41, wherein the bispecific antibody is capable of inducing complement-dependent cytotoxicity on Daudi cells with a half maximal effective concentration (EC50) of less than 10 nM or between 0.5 nM and 1.0 nM.

Embodiment 43. A humanized anti-ICAM1 antibody or ICAM1-binding fragment thereof, comprising CDR sequences at least 90% identical to SEQ ID NO: 56, SEQ ID NO: 57, SEQ ID NO: 58, SEQ ID NO: 59, SEQ ID NO: 60, and SEQ ID NO: 61.

Embodiment 44. The humanized anti-ICAM1 antibody or ICAM1-binding fragment thereof of Embodiment 43, comprising sequences at least 90% identical to SEQ ID NO: 44 and SEQ ID NO: 45.

Embodiment 45. The humanized anti-ICAM1 antibody or ICAM1-binding fragment thereof of Embodiment 43 or 44, comprising one or more ICAM1 binding domains that is capable of binding to the extracellular domain of human ICAM1 with an equilibrium dissociation constant (KD) of less than 1 nM or less than 0.5 nM.

Embodiment 46. The humanized anti-ICAM1 antibody or ICAM1-binding fragment thereof of Embodiment 43 or 44, comprising one or more ICAM1 binding domains that is capable of binding to the extracellular domain of human ICAM1 with an equilibrium dissociation constant (KD) of between 0.02 nM and 10 nM, between 0.05 nM and 5 nM, or between 0.05 nM and 1 nM.

Embodiment 47. The humanized anti-ICAM1 antibody or ICAM1-binding fragment thereof of Embodiment 43 or 44, comprising one or more ICAM1 binding domains that is capable of binding to the extracellular domain of human ICAM1 with an equilibrium dissociation constant (KD) of between 0.1 nM and 0.5 nM.

Embodiment 48. The humanized anti-ICAM1 antibody or ICAM1-binding fragment thereof of any one of Embodiments 43-47, wherein the KD is determined by surface plasmon resonance.

Embodiment 49. A pharmaceutical composition comprising the bispecific antibody of any one of Embodiments 1-42, or the humanized anti-ICAM1 antibody or ICAM1-binding fragment thereof of any one of Embodiments 43-48.

Embodiment 50. The pharmaceutical composition of Embodiment 49, further comprising a pharmaceutically acceptable carrier, an excipient, or any combinations thereof.

Embodiment 51. A method of killing a cell in a subject comprising administering to the subject the bispecific antibody of any one of Embodiments 1-42, or the humanized anti-ICAM1 antibody or ICAM1-binding fragment thereof of any one of Embodiments 43-48, or the pharmaceutical composition of Embodiment 49 or 50, wherein the cell expresses CD38 and ICAM1.

Embodiment 52. The method of Embodiment 51, wherein the cell is lysed.

Embodiment 53. The method of any one of Embodiment 51 or 52, wherein the cell is a tumor cell.

Embodiment 54. A method of reducing growth of a tumor in a subject comprising administering to the subject the bispecific antibody of any one of Embodiments 1-42, or the humanized anti-ICAM1 antibody or ICAM1-binding fragment thereof of any one of Embodiments 43-48, or the pharmaceutical composition of Embodiment 49 or 50, wherein the tumor comprises a cell that expresses CD38 and ICAM1.

Embodiment 55. A method of treating a cancer in a subject comprising administering to the subject the bispecific antibody of any one of Embodiments 1-42, or the humanized anti-ICAM1 antibody or ICAM1-binding fragment thereof of any one of Embodiments 43-48, or the pharmaceutical composition of Embodiment 49 or 50, wherein the cancer comprises a cell that expresses CD38 and ICAM1.

Embodiment 56. The method of Embodiment 55, wherein the cancer comprises a solid tumor or a hematological malignancy.

Embodiment 57. The method of Embodiment 56, wherein the cancer comprises the hematological malignancy.

Embodiment 58. The method of Embodiment 57, wherein the hematological malignancy is multiple myeloma, lymphoma, or Burkitt lymphoma.

Embodiment 59. The method of Embodiment 55, wherein the cancer is a lung cancer or a prostate cancer.

Embodiment 60. The method of any of Embodiments 51-59, wherein the cell expresses at least as much ICAM1 on its surface as an NCI-H2291 cell.

Embodiment 61. The method of Embodiment 60, wherein the cell expresses at least as much CD38 on its surface as an NCI-H2342 cell.

Embodiment 62. The method of any of Embodiment 60 or 61, wherein the cell expresses less CD38 on its surface than a Daudi cell.

Embodiment 63. The method of Embodiment 62, wherein the amount of CD38 on the surface of the cell is less than or equal to the amount of CD38 on the surface of a Raji cell.

Embodiment 64. The method of any of Embodiments 51-63, wherein the ratio of ICAM1 to CD38 on the surface of the cell is greater than the ratio of ICAM1 to CD38 on the surface of a Daudi cell.

Embodiment 65. The method of Embodiment 64, wherein the ratio of ICAM1 to CD38 on the surface of the cell is greater than or equal to the ratio of ICAM1 to CD38 on the surface of a Raji cell.

Embodiment 66. The method of any of Embodiments 51-65, wherein the cell expresses at least 5000, 10000, 150000, 20000, 30000, 50000, 100000, 150000, 200000, 250000, 300000, 400000, or 500000 ICAM1 proteins on its surface.

Embodiment 67. The method of Embodiment 66, wherein the cell expresses at least 50,000 ICAM1 proteins on its surface.

Embodiment 68. The method of any of Embodiments 51-67, wherein the cell expresses at least 100, 200, 300, 400, 500, 1000, 2000, 3000, 4000, or 5000 CD38 proteins on its surface.

Embodiment 69. The method of Embodiment 68, wherein the cell expresses at least 300 CD38 proteins on its surface.

Embodiment 70. The method of Embodiment 68 or 69, wherein the cell expresses less than about 350000, 300000, 250000, 200000, 150000, 100000, 50000, 30000, 20000, 15000, 10000, or 5000 CD38 proteins on its surface.

Embodiment 71. The method of Embodiment 70, wherein the cell expresses less than about 350,000 CD38 proteins on its surface.

Embodiment 72. The method of any of Embodiments 51-71, wherein the ratio of ICAM1 to CD38 on the surface of the cell is at least about 1, 1.5, 2.0, 2.5, 5, 10, 15, 20, 50, 100, or 200.

Embodiment 73. The method of Embodiment 72, wherein the ratio of ICAM1 to CD38 on the surface of the cell is at least about 1.

Embodiment 74. The method of Embodiment 72, wherein the ratio of ICAM1 to CD38 on the surface of the cell is at least about 10.

Embodiment 75. The method of any one of Embodiments 51-74, wherein the subject is a human.

Embodiment 76. A kit comprising the bispecific antibody of any one of Embodiments 1-41, or the humanized anti-ICAM1 antibody or ICAM1-binding fragment thereof of any one of Embodiments 42-48, or the pharmaceutical composition of Embodiment 49 or 50.

### 7. EXAMPLES

These examples are provided for illustrative purposes only and not to limit the scope of the claims provided herein.

### 7.1 Example 1: Tumor Cell Lines Expressing CD38 and ICAM1

Binding and functional activities of the antibodies described in this disclosure were analyzed using the tumor-derived cell lines of Table 1.

TABLE 1

Origin of cell lines

| | | | |
|---|---|---|---|
| Cell Line | Source | Vendor | Catalog number |
| Daudi | Burkitt lymphoma | ATCC | CCL-213 |
| Raji | Burkitt lymphoma | ATCC | CCL-86 |
| KMS-26 | Plasma cell myeloma | JCRB | JCRB1187 |
| HuNS1 | Plasma cell myeloma | ATCC | CRL-8644 |
| HCC44 | Lung adenocarcinoma | KCLB | 70044 |
| NCI-H2291 | Lung adenocarcinoma | ATCC | CRL-5939 |
| NCI-H2342 | Lung adenocarcinoma | ATCC | CRL-5941 |
| DU145 | Prostate carcinoma | ATCC | HTB-81 |

### 7.1.1 Flow Cytometry Binding Assay

Surface expression of CD38 and ICAM1 on the transformed cell lines was quantified by flow cytometry. Harvested cells were centrifuged at 2000 rpm for 5 min, resuspended in 10 - 15 ml ice-cold culture medium, and then counted. Then, 3×10⁶ cells were resuspended per ml of blocking buffer (PBS plus 2% FBS). 100 µl of the cell suspension was dispensed into each well of a 96-well plate and incubated at room temperature for 10-20 min. For Fc receptor-expressing cells, 5 µl human Fc block (BD Biosciences, Cat#: 564220) was added and incubated for 15 minutes. While incubating, purified antibodies were diluted to the desired dilution with blocking buffer. After a 10-20 minutes incubation, cells were centrifuged for 5 minutes at 2000 rpm in a refrigerated centrifuge. Blocking buffer was aspirated, and the cells were resuspended in 100 µl/well diluted antibodies and incubated for 1 hour at 4° C. The cells were then washed 3 times with PBS plus 2% FBS. After the third wash, the cells were resuspended in 100 µl 1:500 diluted Alexa Fluor 488 labeled Mouse anti-Human IgG1 Fc secondary antibody (Invitrogen, Cat#: A10631) and incubated for 1 hour at 4° C. in the dark. The cells were then washed 3 times with 200 µl PBS by centrifuging at 2000 rpm for 5 min. After the last wash, the cells were resuspended in 300 µl cold PBS and analyzed on a FACSVerse™ (BD Biosciences) flow cytometer. The relative expression levels of CD38 and ICAM1 in nine cell lines are shown in Table 2.

TABLE 2

Surface expression of CD38 and ICAM1 on representative cell lines as measured by flow cytometry. MFI = mean fluorescence index

| Cell Line | CD38 (MFI) | ICAM1 (MFI) |
|---|---|---|
| Daudi | 46116 | 1158 |
| Raji | 35114 | 5489 |
| KMS-26 | 3920 | 3433 |
| HuNS1 | 1016 | 1817 |
| HCC44 | 689 | 2734 |
| NCI-H2291 | 239 | 500 |
| NCI-H2342 | 41 | 912 |
| DU145 | 60 | 2539 |

### 7.2 Example 2: Production of CD38 and ICAM1 Antigens

The full-length extracellular domains (ECD) of human CD38 and ICAM1 were cloned into pCDNA3.1 vectors comprising a 6XHis affinity tag (SEQ ID NO: 62), a TEV cleavage site, and an AviTag for direct biotinylation. The amino acid sequences of these AviTagged antigens (post-cleavage) are shown in Table 3. Transfected Expi293 cultures expressing the CD38 and ICAM1 proteins were centrifuged for 10 min at 2000 rpm and 4° C., and the supernatants were collected. Ni-NTA resin was pre-equilibrated with E buffer (137 mM NaCl, 2.7 mM KCl, 10 mM Na₂HPO₄, 2 mM KH₂PO₄, pH 7.4), incubated for 2 hours on a rotator with supernatant at 4° C., and then poured into a column. The column was washed with I-20 buffer (137 mM NaCl, 2.7 mM KCl, 10 mM Na₂HPO₄, 2 mM KH₂PO₄, pH 7.4, 20 mM imidazole) until no signal was observed by G-250. The target protein was eluted with 5 CV of I-250 buffer (137 mM NaCl, 2.7 mM KCl, 10 mM Na₂HPO₄, 2 mM KH₂PO₄, pH 7.4, 250 mM imidazole). The eluate was dialyzed against E buffer and then cleaved with TEV protease overnight at 4° C. at a 1:10 ratio of protease to protein.

Gel filtration was utilized to assay for aggregation. A small aliquot of each isolated ECD was loaded onto a 24 ml Superdex™ 200 column (10/300GL, GE) that had been preequilibrated with E buffer. The antigens eluted as monomers. The remainder of the protein was dialyzed into storage buffer (137 mM NaCl, 2.7 mM KCl, 10 mM Na₂HPO₄, 1.76 mM KH₂PO₄, 6% Sucrose, pH 7.4), concentrated in an ultra-filtration tube with a 30 KDa molecular weight cutoff, snap-frozen with liquid N₂, and stored at -80° C.

TABLE 3

Antigen sequences

| | | |
|---|---|---|
| SEQ ID NO: | ANTIGEN NAME | SEQUENCE |
| 1 | Avi-human CD38 ECD | SGLNDIFEAQKIEWHEVPRWRQQWSGPGTTKRFPETVLARCVKYTEIH PEMRHVDCQSVWDAFKGAFISKHPCNITEEDYQPLMKLGTQTVPCNKI LLWSRIKDLAHQFTQVQRDMFTLEDTLLGYLADDLTWCGEFNTSKINY QSCPDWRKDCSNNPVSVFWKTVSRRFAEAACDVVHVMLNGSRSKIFDK NSTFGSVEVHNLQPEKVQTLEAWVIHGGREDSRDLCQDPTIKELESII SKRNIQFSCKNIYRPDKFLQCVKNPEDSSCTSEI |
| 2 | Human ICAM1 ECD-Avi | QTSVSPSKVILPRGGSVLVTCSTSCDQPKLLGIETPLPKKELLLPGNN RKVYELSNVQEDSQPMCYSNCPDGQSTAKTFLTVYWTPERVELAPLPS WQPVGKNLTLRCQVEGGAPRANLTVVLLRGEKELKREPAVGEPAEVTT TVLVRRDHHGANFSCRTELDLRPQGLELFENTSAPYQLQTFVLPATPP QLVSPRVLEVDTQGTVVCSLDGLFPVSEAQVHLALGDQRLNPTVTYGN DSFSAKASVSVTAEDEGTQRLTCAVILGNQSQETLQTVTIYSFPAPNV ILTKPEVSEGTEVTVKCEAHPRAKVTLNGVPAQPLGPRAQLLLKATPE |
| | | DNGRSFSCSATLEVAGQLIHKNQTRELRVLYGPRLDERDCPGNWTWPE NSQQTPMCQAWGNPLPELKCLKDGTFPLPIGESVTVTRDLEGTYLCRA RSTQGEVTRKVTVNVLSPRYEGGSGGSGLNDIFEAQKIEWHEENLYFQ |

### 7.3 Example 3: CD38 and ICAM 1 Monoclonal Antibodies

Rabbit monoclonal antibodies specific for CD38 or ICAM1 were generated as described in PCT/US2019/061884, which is incorporated by reference in its entirety.

Rabbits were immunized four times with 10⁸ Daudi cells (for CD38 antibodies) or CHO-G10 cells (a stable clone expressing ICAM1). Serum titers were assayed by enzyme-linked immunosorbent assay (ELISA) using recombinant ICAM1 and CD38 proteins. Target recognition was further assayed by flow cytometry. For each target, a rabbit with a high ELISA titer and a strong flow cytometry signal was boosted 4 days ahead of splenectomy via IV injection of 400 µg of recombinant ICAM1 or CD38. For each target, 1.2E8 freshly isolated splenocytes were cultured overnight in customized B cell medium before sorting. Splenocytes were processed using the SMabTM platform to enrich antigen-recognizing B cells. FACS-sorted B cells were cultured at 1 cell/well in a 96-well plate for 10-14 days.

Clones positive for antigen recognition were identified using a direct antigen ELISA. To identify monoclonal antibodies suitable for FACS analysis, initial positive clones were screened against CHO cell lines expressing CD38 (CHO-F10) and ICAM1 (CHO-G10). Untransfected CHO cells were used as a negative control. FACS-positive mAb clones were further confirmed using linear expression module (LEM) supernatants from HEK293F cells transiently expressing recombinant IgG genes recovered from the initial positive clones.

Stable cell lines expressing the CD38 and ICAM1 antigens were maintained in selection medium with antibiotics to maintain transgene expression. CHO-G10 cells were grown in F12 medium supplemented with 10% FBS and 5 ug/ml puromycin. CHO-F10 cells were grown in F12 medium supplemented with 10% FBS and 5 ug/ml puromycin and 2 mg/ml neomycin.

Chimeric antibodies with rabbit variable domains and human constant domains were generated by fusing the VH domain of the anti-CD38 and anti-ICAM1 monoclonal antibodies to a human IgG1 constant region and the VL domain of the anti-CD38 and anti-ICAM1 monoclonal antibodies to a human Kappa constant region. Because the human Kappa constant region does not have a cysteine corresponding to the cysteine in the rabbit Kappa constant region that forms an interdomain disulfide bond with the rabbit VL (Cys80-Cys171), Cys80 in the VL region was replaced with Ser to eliminate the free cysteine.

Tables 4-6 present sequences of exemplary chimeric anti-CD38 and anti-ICAM1 monoclonal antibodies discovered by these methods, including their heavy and light chains, variable domains, and complementarity-determining regions (CDRs).

TABLE 4

Sequences of anti-CD38 chimeric monoclonal antibody 18E4

| DOMAIN | SEQUENCE | SEQ ID NO: |
|---|---|---|
| HC | QSLEESGGDLVKPGASLTLTCTASGFSFNNYWICWIRQAPGKGLEWV ACIYSPSGDIKYYANWAKGRFTVSKTSSTTVTLQMTSLTGADTATYF CARELSGSSYEGYFESWGPGTLVTVSSASTKGPSVFPLAPSSKSTSGG TAALGCLVKDYFPEPVTVSWNSGALTSGVHTFPAVLQSSGLYSLSSV VTVPSSSLGTQTYICNVNHKPSNTKVDKKVEPKSCDKTHTCPPCPAP ELLGGPSVFLFPPKPKDTLMISRTPEVTCVVVDVSHEDPEVKFNWYV DGVEVHNAKTKPREEQYNSTYRVVSVLTVLHQDWLNGKEYKCKVS NKALPAPIEKTISKAKGQPREPQVYTLPPSRDELTKNQVSLTCLVKGF YPSDIAVEWESNGQPENNYKTTPPVLDSDGSFFLYSKLTVDKSRWQQ GNVFSCSVMHEALHNHYTQKSLSLSPGK | 3 |
| LC | AQVLTQTPSSVSAAVGGTVTINCQCSQSVYGHNWLAWYQHKPGQP PKLLMYRASNLASGVPSRFKGSGSGSQFTLTIGEVQSDDAATYYCQG YYNGGSYAFGGGTEVVVRRTVAAPSVFIFPPSDEQLKSGTASVVCLL NNFYPREAKVQWKVDNALQSGNSQESVTEQDSKDSTYSLSSTLTLS KADYEKHKVYACEVTHQGLSSPVTKSFNRGEC | 4 |
| VH | QSLEESGGDLVKPGASLTLTCTASGFSFNNYWICWIRQAPGKGLEWV ACIYSPSGDIKYYANWAKGRFTVSKTSSTTVTLQMTSLTGADTATYF CARELSGSSYEGYFESWGPGTLVTVSS | 5 |
| VL | AQVLTQTPSSVSAAVGGTVTINCQCSQSVYGHNWLAWYQHKPGQP PKLLMYRASNLASGVPSRFKGSGSGSQFTLTIGEVQSDDAATYYCQG YYNGGSYAFGGGTEVVVR | 6 |
| HC CDR1 | NYWIC | 7 |
| HC CDR2 | CIYSPSGDIKYYANWAKG | 8 |
| HC CDR3 | ELSGSSYEGYFES | 9 |
| LC CDR1 | QCSQSVYGHNWLA | 10 |
| LC CDR2 | RASNLAS | 11 |
| LC CDR3 | QGYYNGGSYA | 12 |

TABLE 5

Sequences of anti-ICAM1 chimeric monoclonal antibody 11F2

| DOMAIN | SEQUENCE | SEQ ID NO: |
|---|---|---|
| HC | QSVEESGGGLVKPGGTLTLTCTVSGFSLSSHAMGWVRQAPGKGLEY IGIIGSSDRTYYASWAKGRFTISKTSTTVDLKITSPTTEDTATYFCVRD PYDSYDDGYRLWGPGTLVTVSSASTKGPSVFPLAPSSKSTSGGTAAL GCLVKDYFPEPVTVSWNSGALTSGVHTFPAVLQSSGLYSLSSVVTVP SSSLGTQTYICNVNHKPSNTKVDKKVEPKSCDKTHTCPPCPAPELLG GPSVFLFPPKPKDTLMISRTPEVTCVVVDVSHEDPEVKFNWYVDGVE VHNAKTKPREEQYNSTYRVVSVLTVLHQDWLNGKEYKCKVSNKAL PAPIEKTISKAKGQPREPQVYTLPPSRDELTKNQVSLTCLVKGFYPSDI AVEWESNGQPENNYKTTPPVLDSDGSFFLYSKLTVDKSRWQQGNVF SCSVMHEALHNHYTQKSLSLSPGK | 13 |
| LC | AYDMTQTPASVEVAVGGTVTIKCQASQSIYRYLSWYQKKPGQRPKF LIYDASKLASGVPSRFEGSGSGTEFTLTISGAQSDDAATYYCQQAYSS GSIDNAFGGGTEVVVKRTVAAPSVFIFPPSDEQLKSGTASVVCLLNNF YPREAKVQWKVDNALQSGNSQESVTEQDSKDSTYSLSSTLTLSKAD YEKHKVYACEVTHQGLSSPVTKSFNRGEC | 14 |
| VH | QSVEESGGGLVKPGGTLTLTCTVSGFSLSSHAMGWVRQAPGKGLEY IGIIGSSDRTYYASWAKGRFTISKTSTTVDLKITSPTTEDTATYFCVRD PYDSYDDGYRLWGPGTLVTVSS | 15 |
| VL | AYDMTQTPASVEVAVGGTVTIKCQASQSIYRYLSWYQKKPGQRPKF LIYDASKLASGVPSRFEGSGSGTEFTLTISGAQSDDAATYYCQQAYSS GSIDNAFGGGTEVVVK | 16 |
| HC CDR1 | SHAMG | 17 |
| HC CDR2 | IIGSSDRTYYASWAKG | 18 |
| HC CDR3 | DPYDSYDDGYRL | 19 |
| LC CDR1 | QASQSIYRYLS | 20 |
| LC CDR2 | DASKLAS | 21 |
| LC CDR3 | QQAYSSGSIDNA | 22 |

TABLE 6

Sequences of anti-ICAM1 chimeric monoclonal antibody 8B12

| DOMAIN | SEQUENCE | SEQ ID NO: |
|---|---|---|
| HC | QSVEESGGGLVKPGGTLTLTCTVSGFSLSTHAMGWVRQAPGKGLEY IGIIGSSDRTYYASWAKGRFTISKTSTTVDLKITSPTTEDTATYFCVRD PYDSFDDGYRLWGPGTLVTVSSASTKGPSVFPLAPSSKSTSGGTAAL GCLVKDYFPEPVTVSWNSGALTSGVHTFPAVLQSSGLYSLSSVVTVP SSSLGTQTYICNVNHKPSNTKVDKKVEPKSCDKTHTCPPCPAPELLG GPSVFLFPPKPKDTLMISRTPEVTCVVVDVSHEDPEVKFNWYVDGVE VHNAKTKPREEQYNSTYRVVSVLTVLHQDWLNGKEYKCKVSNKAL PAPIEKTISKAKGQPREPQVYTLPPSRDELTKNQVSLTCLVKGFYPSDI AVEWESNGQPENNYKTTPPVLDSDGSFFLYSKLTVDKSRWQQGNVF SCSVMHEALHNHYTQKSLSLSPGK | 23 |
| LC | AYDMTQTPASVEVAVGGTVTIKCQASQSIYSYLSWYQQKPGQRPKF LIYDASKVASGVPSRFKGSGSGTEFTLTISAVQSDDAATYYCQQAYS SSNVDNAFGGGTEVMVKRTVAAPSVFIFPPSDEQLKSGTASVVCLLN | 24 |
| | NFYPREAKVQWKVDNALQSGNSQESVTEQDSKDSTYSLSSTLTLSK ADYEKHKVYACEVTHQGLSSPVTKSFNRGEC | |
| VH | QSVEESGGGLVKPGGTLTLTCTVSGFSLSTHAMGWVRQAPGKGLEY IGIIGSSDRTYYASWAKGRFTISKTSTTVDLKITSPTTEDTATYFCVRD PYDSFDDGYRLWGPGTLVTVSS | 25 |
| VL | AYDMTQTPASVEVAVGGTVTIKCQASQSIYSYLSWYQQKPGQRPKF LIYDASKVASGVPSRFKGSGSGTEFTLTISAVQSDDAATYYCQQAYS SSNVDNAFGGGTEVMVK | 26 |
| HC CDR1 | THAMG | 27 |
| HC CDR2 | IIGSSDRTYYASWAKG | 28 |
| HC CDR3 | DPYDSFDDGYRL | 29 |
| LC CDR1 | QASQSIYSYLS | 30 |
| LC CDR2 | DASKVAS | 31 |
| LC CDR3 | QQAYSSSNVDNA | 32 |

### 7.4 Example 4: Binding and Functional Evaluation of Chimeric Anti-CD38 and Anti-ICAM1 Antibodies

### ELISA Binding Assay

A 96-well plate was coated overnight at 4° C. with 1 µg/ml recombinant CD38 or ICAM1. After washing 3 times, the plate was blocked with 300 µl 1% BSA in PBST at 37° C. for 1 hour. Serially diluted antibodies were added and incubated at 37° C. for 1 hour. The plate was then washed 4 times with PBST and incubated with 1:5000 diluted 2nd antibody (Sigma, Cat# A0293) at 37° C. for 1 hour. The plate was washed again 4 times with PBST, incubated with TMB substrate for 15 min at room temperature, terminated with 1N HCl, and then read at 450 nM. Chimeric antibody 18E4 bound to CD38 with a half maximal effective concentration (EC50 or EC₅₀) of approximately 50 pM. Chimeric antibodies 11F2 and 8B12 bound to ICAM1 with EC50s of approximately 50 pM.

### Flow Cytometry Binding Assay

Binding of the chimeric anti-CD38 and anti-ICAM1 antibodies to proteins on the surface of tumor cells was determined by flow cytometry, as described above. Chimeric antibodies 18E4 and anti-CD38 BMK bound to CD38 on Daudi cells with an EC50 of 0.3 - 1.0 nM. Chimeric antibodies 11F2, and 8B12 bound to ICAM1on DU145 cells with an EC50 of 1.0 - 1.5 nM.

### Surface Plasmon Resonance Binding Assay

Surface plasmon resonance (SPR) is a more accurate and sensitive assay for binding kinetics and affinities than an ELISA. Furthermore, binding kinetics measurements of the anti-CD38 and anti-ICAM1 antibodies were not affected by antibody valency in the SPR assays because antibodies were immobilized and challenged with monomeric antigens. SPR was performed on a Biacore 8K instrument (GE Healthcare). Antibodies were captured on Biacore Series S CM5 sensor chips coated with a monoclonal mouse anti-human IgG (Fc) antibody (GE Healthcare human antibody capture kit). Serial 3-fold dilutions of antigen (CD38 ECD or ICAM1 ECD) were injected at a flow rate of 30 µl/min. Each sample was analyzed with 1 min association and 10 min dissociation at room temperature (25° C.). After each injection, the chip was regenerated using 3 M MgCl₂. A 1:1 Langmuir model of simultaneous fitting of kₒₙ and k_{off} was used for kinetics analysis. Chimeric antibody 18E4 bound to human CD38 with a KD of 0.1 to 0.5 nM. Chimeric antibodies 11F2 and 8B12 bound to human ICAM1 with a KD of 0.1 to 0.5 nM.

### Functional Activities

### Complement-Dependent Cytotoxicity

To assay for complement-dependent cytotoxicity or CDC, the complement, antibodies or target cells were diluted in cell culture medium without FBS. Target cells were harvested in logarithmic growth phase and washed twice with cell culture medium without FBS. Cell density was adjusted to 4×10⁵ /ml and 50 µL of cell suspension were added to each well of the assay plate. Antibodies were prepared at 4X of final concentrations. 25 µL of serially diluted antibodies were then added to each well of the assay plate and incubated at 37° C. for 30 mins. 25 µL of diluted human serum was then added to each well of the assay plate. The working concentration of complement was 10%. All the media used for complement, antibody and target cells dilution was serum-free. The assay plates were incubated at 37° C. for 4 hrs. Then 50 µL CellTiter-Glo Luminescent buffer was added to each well, mixed extensively on an orbital shaker for 2 minutes to induce cell lysis. The plate was then incubated at room temperature for 10 minutes to stabilize the luminescent signal. Luminescence was measured by SpectraMax M5. Data was analyzed by GraphPad prism 5 using nonlinear regression fit. Chimeric anti-CD38 antibody 18E4 had potent CDC activity on Daudi cells, with an EC50 of 1-2 nM.

### Antibody-Dependent Cell Cytotoxicity

To assay for antibody-dependent cellular cytotoxicity (ADCC), target cells were washed once with balanced salt solution or culture medium, and cell numbers were adjusted to 1×10⁶ cells/ml. 2 µL of BATDA fluorescence enhancing ligand (Perkin Elmer, Cat# C136-100) was added to each mL of cells and incubated for 20 min at 37° C. in a cell incubator. After incubation, cells were centrifuged, culture medium was aspirated. The labeled cells were washed 4 times with PBS. After the final wash, cells were re-suspended in culture medium and adjusted to 5 × 10⁴ cell /ml. 200 µL (1× 10⁴ cells) of the cell suspension was added to each well of a 96-well plate. Background release was determined by withdrawing an aliquot of the labeled target cells, centrifuge and supernatant was transferred into an empty well. Antibodies were serially diluted with RPMI-1640 containing 10% FBS. 50 µL of serially-diluted antibodies were added to assay plate containing target cell and incubated at 37° C., 5% CO₂ for 5-10 min. Effector cells NK92/CD16a176V or freshly isolated PBMC were harvested and suspended in RPMI-1640 containing 10% FBS. 50 ul/well effector cells were added to each well of assay plate at different ET ratio. Set up controls: target spontaneous (target cell+100 µL medium); target maximum (target cell+100 µL medium+10 µL lysis buffer); background (100 µL the labeled target cell supernatant and 100 µL dilution medium). The plates were incubated in a humidified 5% CO₂ atmosphere at 37° C. for 2 hours. At the end of incubation, 10 µL of Lysis Buffer (Perkin Elmer, Cat# 4005-0010) was added to the maximum release well. The plates were centrifuged for 5 min at 500 g. 20 µL of the supernatant from each well was transferred to a flat-bottom detection plate. 200 µL of Europium Solution (Perkin Elmer, Cat# C135-100) was then added to each well of the detection plate. The plate was shaken at 250 rpm for 15 min at room temperature, and the fluorescence was then measured in a time-resolved fluorometer within 5 hrs.

Chimeric anti-CD38 antibody 18E4 had potent ADCC activity on Daudi and HuNS1 cells, with an EC50 of between 0.1 and 0.5 pM for Daudi cells. The 11F2 and 8B12 chimeric anti-ICAM1 antibodies had potent ADCC activity in DU145 cells, which have high ICAM1 expression, with EC50s of between 0.01 and 0.1 nM

### 7.5 Example 5: Humanization

The 18E4, 11F2, and 8B12 antibodies were humanized by grafting their CDR sequences into a human framework. The CDR graft variants had reduced affinity for their antigens compared to the chimeric antibodies, and they contained sequences that might reduce their stability. Thus, they were further engineered by replacing human framework residues with the corresponding rabbit framework residues at positions that contribute to binding and replacing potential chemical hot spots with residues that do not have chemical liabilities.

### 7.5.1 Humanization of the Anti-CD38 Antibody, 18E4

The sequences of 18E4 variable domains were aligned to sequences from the international ImMunoGeneTics information system® (IMGT®) library to identify human germline VH and VL-kappa sequences with homology to the HV and VL chains of 18E4. IGKV1-05 was chosen as the VL human germline acceptor sequence. IGHV3-23 was chosen as the VH human germline acceptor sequence.

The simple graft variant h18E4-1 had substantially reduced affinity for CD38 compared to the 18E4 chimeric antibody. To restore its affinity, selected amino acids in the framework regions were changed back to the rabbit sequence. Three light chain Vernier positions M₄, I₄₈ and L₇₈ (FIG. 1A) and eight heavy chain Vernier positions V₃₇, S₄₉, S₆₂, V₆₃, I₆₉, R₇₁, L₇₈ and K₉₄ (FIG. 1B) were evaluated.

Additional variants were constructed to reduce the potential chemical instability of the final clone. A potential deamidation site (NG) in LC CDR3 was mutated to AG, SG, or NA (FIG. 1A), and cysteine residues in LC CDR1, HC CDR1, and HC CDR2 were mutated to serine or alanine (FIGS. 1A-1B).

The mutated 18E4 heavy and light chain variants of FIGS. 1A-1B were combined to generate humanized 18E4 antibody variants as shown in Table 7.

TABLE 7

Heavy and light chain composition of humanized 18E4 variants

| | | | | | | | | | |
|---|---|---|---|---|---|---|---|---|---|
| | L1 | L2 | L3 | L4 | L5 | L6 | L7 | L8 | L9 |
| H1 | h18E4-1 | | | | | | | | |
| H2 | | h18E4-2 | h18E4-3 | h18E4-4 | h18E4-5 | | | | |
| H3 | | h18E4-6 | h18E4-7 | h18E4-8 | h18E4-9 | h18E4-12 | h18E4-13 | h18E4-14 | |
| H4 | | | | | h18E4-11 | | | | |
| H5 | | | | | h18E4-15 | | | | |
| H6 | | | | | h18E4-16 | | h18E4-19 | h18E4-18 | h18E4-17 |

The binding kinetics of humanized 18E4 and h18E4 variants with back mutations in the framework regions were evaluated by SPR. CDR graft variant h18E4-1 bound to human CD38 with a >10-fold higher KD than chimeric 18E4. Adding back framework residues improved the affinity of variants h18E4-2 through h18E4-8 to approximately the same affinity (about 50% -200%) as chimeric 18E4. h18E4-9, which has 3 LC back mutations and 8 HC back mutations, had a lower KD than chimeric 18E4. Adding back amino acid residues in the framework regions also improved the affinity of humanized 18E4 variants for cell surface CD38 and increased their ADCC activity. In particular, h18E4-9 and chimeric 18E4 had comparable affinity for CD38 on the surface of Daudi cells and had comparable ADCC activity on HuNS 1 target cells, with an EC50 of 0.01 -0.05 nM.

Having established the good binding and functional properties of h18E4-9, this variant was further mutated to eliminate potential chemical instability sites. Among a set of mutations designed to eliminate a potential NG deamidation site in the light chain, the asparagine to serine mutation of h18E4_L7 (as incorporated into h18E4-13) had the least deleterious effects on binding affinity as determined by surface plasmon resonance. The h18E4_H3 heavy chain of h18E4-13 was further mutated to eliminate cysteine residues in the CDR1 and CDR2 domains to generate h18E4_H6, which was incorporated into h18E4-19. In an ADCC assay on Daudi cells, h18E4-19 had potent activity, with an EC50 concentration (0.05 - 0.1 pM) similar to chimeric 18E4 and slightly more potent than the benchmark anti-CD38 antibody daratumumab (0.1 - 0.5 pM).

### Alanine Scanning

Alanine scanning variants of the humanized h18E4-19 antibody were generated to identify CDR3 residues important for binding to human CD38. Alanine mutants were systematically generated by mutating each residue of the heavy and light chain CDR3 regions to alanine, as shown in FIGS. 2A - 2B. Each mutant variant was tested for binding to CD38 by ELISA, as shown in Table 8. The variants with EC50 values of 0.02 nM - 0.1 nM (class A) identify CDR3 positions that can be mutated without substantially inhibiting binding to CD38.

TABLE 8

Binding of humanized 18E4 alanine variants to the human CD38 ECD, as determined by ELISA. Amino acid numbering is based on the Kabat numbering system. EC50 ranges for huCD38 - A: 0.02 nM - 0.1 nM; B: 0.1 nM - 0.5 nM; C: >0.5 nM

| | |
|---|---|
| Anti-CD38 Antibody | EC50 (nM) |
| hu18E4-19 | A |
| LC Q89A | A |
| LC G90A | B |
| LC Y91A | C |
| LC Y92A | A |
| LC S93A | B |
| LC G94A | C |
| LC G95A | A |
| LC S95aA | A |
| LC Y96A | B |
| HC E95A | C |
| HC L96A | A |
| HC S97A | A |
| HC G98A | A |
| HC S99A | A |
| HC S100A | A |
| HC Y100aA | A |
| HC E100bA | C |
| HC G100cA | B |
| HC Y100dA | C |
| HC F100eA | A |
| HC E101A | A |
| HC S102A | A |
| hIgG1 control | NB |

*NB: no binding, binding too low to quantify

### 7.5.2 Humanization of the Anti-ICAM1 Antibodies 8B12 and 11F2

### Humanization of the Anti-ICAM1 Antibody, 8B12

The sequences of the 8B12 heavy and light chain variable domains were aligned to sequences from the international ImMunoGeneTics information system® (IMGT®) library to identify homologous human germline VH and VL-kappa sequences. For 8B12 VL, human germline IGKV1-05 was chosen as the acceptor sequence. For 8B12 VH, human germline IGHV3-23 was chosen as the acceptor sequence.

The CDR graft variant h8B 12-1 had substantially reduced binding to ICAM1 on the surface of Daudi cells and in a SPR assay. To restore affinity, selected amino acids at the following Vernier positions in the framework regions were evaluated: light chain I₂, L₄₆, L₇₈, F₈₃ and I₁₀₆ (FIG. 3A) and heavy chains L₄, A₂₄, W₄₇, V₄₈, S₄₉, S₆₂, V₆₃, R₇₁, L₇₈, M₈₂, L_{82c}, A₉₃ and K₉₄ (FIG. 3B). The mutated 8B12 heavy and light chain variants of FIGS. 3A-3B were combined to generate humanized 8B12 antibody variants, as shown in Table 9.

TABLE 9

Heavy and light chain composition of humanized 8B12 variants

| | | | | |
|---|---|---|---|---|
| | L1 | L2 | L2b | L2c |
| H1 | h8B12-1 | | | |
| H2 | h8B12-2 | h8B12-3 | | |
| H3 | h8B12-4 | h8B12-5 | | |
| H4 | h8B12-6 | h8B12-7 | | |
| H3b | | h8B12-16 | h8B12-17 | h8B12-18 |
| H4b | | h8B12-19 | h8B12-20 | h8B12-21 |

A flow cytometry assay revealed that the back mutations of h8B12-2 through h8B12-7 improved binding to ICAM1 on the surface of Daudi cells (EC50: 0.08 nM - 0.2 nM) compared to the CDR graft variant h8B12-1 (EC50: 0.2 nM - 0.5 nM). However, h8B12-2 through h8B12-7 still had lower affinity than chimeric 8B12 (EC50: 0.04 nM - 0.08 nM). These results were confirmed by surface plasmon resonance. h8B12-2 through h8B12-7 had affinity constants (KD) ranging from 0.3 nM - 1 nM. This represented a >10-fold improvement over h8B12-1 but was still weak binding compared to chimeric 8B12 (KD: 1 nM - 2 nM). An evaluation of additional framework region variants led to h8B12-21, which had further improved binding properties. h8B12-21 also had improved ADCC activity compared to h8B12-4, h8B12-6, h8B12-7 and h8B12-19.

### Humanization of the Anti-ICAM1 Antibody, 11F2

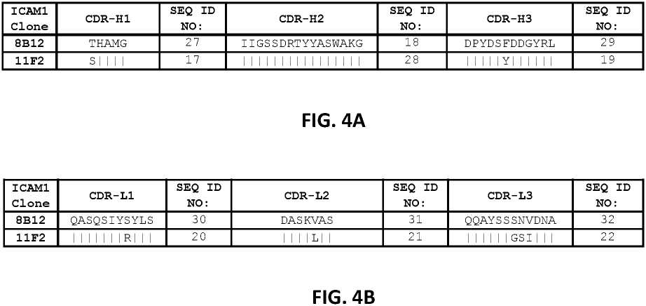

The 11F2 clone shares high sequence similarity with 8B12, as shown in FIG. 4. 11F2 humanization started by grafting the 11F2 CDRs into the framework region of h8B12-21. Then, light chain residues I₂, F₈₃, and I₁₀₆ (FIG. 5A) and heavy chain residue W₄₇ (FIG. 5B) were mutated back to human residues to evaluate their contributions to antigen binding. Additional mutations were generated to polish a chemical liable site (D_{100b}G_{100c}) in the 11F2 CDRH3 regions. The resulting heavy and light chains were combined into anti-ICAM1 antibodies for further evaluation, as shown in Table 10.

TABLE 10

Heavy and light chain composition of humanized 11F2 variants

| | | | |
|---|---|---|---|
| | L1 | L2 | L3 |
| H1 | h11F2-1 | h11F2-3 | |
| H2 | h11F2-4 | h11F2-2 | |
| H3 | | | h11F2-5 |
| H4 | | | h11F2-6 |
| H5 | | | h11F2-7 |
| H6 | | | h11F2-8 |
| H7 | | | h11F2-9 |

h11F2-1 through h11F2-4 had potent ADCC activity on Daudi cells, with EC50 values of 0.05 nM - 0.2 nM. h11F2-5 through h11F2-9 all had binding affinities (KD) similar to h8B12-21 (0.1 nM - 0.5 nM), as determined by surface plasmon resonance.

### 7.6 Example 6: Humanized Bispecific Antibodies in Different Formats

Humanized anti-CD38 (h18E4-19) and anti-ICAM1 (h11F2-6) binding domains were joined to form bispecific antibodies. Two formats of bispecific antibodies were constructed. One had three polypeptide chains with a CD38 scFv domain. The other had four polypeptide chains and used the T-Cell Receptor (TCR) constant domains to distinguish the CD38 binding arm from the ICAM1 binding-arm. The Fc domains of both bispecific formats were engineered with knob-into-hole mutations to inhibit homodimer assembly. Various knob-to-holes formats and other methods for constructing bispecific antibodies are described in PCT/US19/61884 and Carter, J. Immunol. Protein Engineering 9(7) 617-621, 1996.

### 7.6.1 Construction of αCD38(scFv) X αICAM1

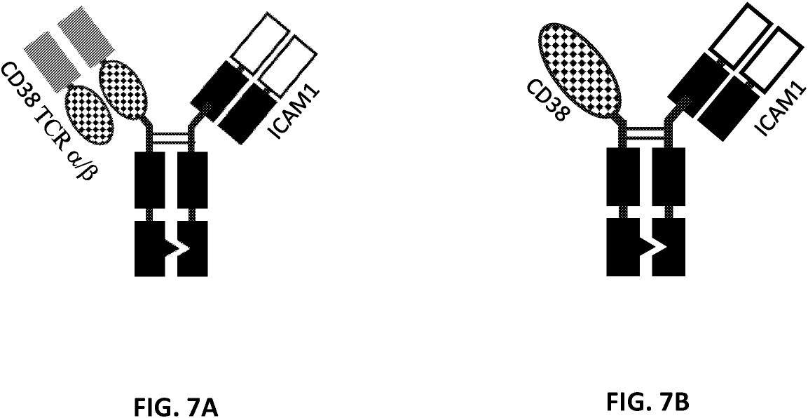

αCD38(scFv) x αICAM1 is a bispecific antibody with a single chain CD38 binding domain (scFv) derived from h18E4-19, an ICAM1 binding domain derived from h11F2-6, and an IgG1 Fc domain with knob-into-hole mutations to facilitate assembly of bispecific antibodies. Incorporation of an scFv domain into one arm of a bispecific antibody inhibits the assembly of nonspecific antibodies (FIG. 7B). Sequences of the polypeptide components of αCD38(scFv) x αICAM1 are shown in Table 11. Sequences of the Fc domains of αCD38(scFv) x αICAM1, including knob and hole mutations, are shown in Table 12.

TABLE 11

Polypeptide chains of αCD38(scFv) x αICAM1

| SEQ ID NO. | Name | Sequence |
|---|---|---|
| 33 | Three-Chain h11F2-6 HC | EVQVLESGGGLVQPGGSLRLSCAVSGFSLSSHAMGWVRQAPGKGLE WIGIIGSSDRTYYASWAKGRFTISKDNSKNTVYLQINSPRAEDTAV YYCVRDPYDSYDEGYRLWGQGTLVTVSSASTKGPSVFPLAPSSKST SGGTAALGCLVKDYFPEPVTVSWNSGALTSGVHTFPAVLQSSGLYS LSSVVTVPSSSLGTQTYICNVNHKPSNTKVDKKVEPKSCDKTHTCP PCPAPELLGGPSVFLFPPKPKDTLMISRTPEVTCVVVDVSHEDPEV KFNWYVDGVEVHNAKTKPREEQYNSTYRVVSVLTVLHQDWLNGKEY KCKVSNKALPAPIEKTISKAKGQPREPQVYTLPPSRDELTKNQVSL WCLVKGFYPSDIAVEWESNGQPENNYKTTPPVLDSDGSFFLYSKLT VDKSRWQQGNVFSCSVMHEALHNHYTQKSLSLSPGK |
| 34 | Three-Chain h11F2-6 LC | DIQMTQSPSTLSASVGDRVTITCQASQSIYRYLSWYQQKPGKAPKF LIYDASKLASGVPSRFSGSGSGTEFTLTISSVQPDDFATYYCQQAY SSGSIDNAFGGGTKVEIKRTVAAPSVFIFPPSDEQLKSGTASVVCL LNNFYPREAKVQWKVDNALQSGNSQESVTEQDSKDSTYSLSSTLTL SKADYEKHKVYACEVTHQGLSSPVTKSFNRGEC |
| 35 | scFv-h18E4-19-Fc | DIQLTQSPSTLSASVGDRVTITCRSSQSVYGHNWLAWYQQKPGKAP KLLMYRASNLASGVPSRFSGSGSGTEFTLTISSVQPDDFATYYCQG YYSGGSYAFGGGTKVEIKGGGGSGGGGSGGGGSEVQLLESGGGLVQ PGGSLRLSCAASGFSFNNYWIIWIRQAPGKGLEWVAAIYSPSGDIK YYADWAKGRFTVSKDNSKNTVYLQMNSLRAEDTAVYYCARELSGSS YEGYFESWGQGTLVTVSSGGSGGSGGASKTHTCPPCPAPELLGGPS VFLFPPKPKDTLMISRTPEVTCVVVDVSHEDPEVKFNWYVDGVEVH NAKTKPREEQYNSTYRVVSVLTVLHQDWLNGKEYKCKVSNKALPAP IEKTISKAKGQPREPQVYTLPPSRDELTKNQVSLSCAVKGFYPSDI AVEWESNGQPENNYKTTPPVLDSDGSFFLVSKLTVDKSRWQQGNVF SCSVMHEALHNHYTQKSLSLSPGK |

TABLE 12

IgG1 domain sequences of αCD38(scFv) x αICAM1 with knobs and holes

| SEQ ID NO. | Name | Sequence |
|---|---|---|
| 36 | hFc-knob | EPKSCDKTHTCPPCPAPELLGGPSVFLFPPKPKDTLMISRTPEVTC VVVDVSHEDPEVKFNWYVDGVEVHNAKTKPREEQYNSTYRVVSVLT VLHQDWLNGKEYKCKVSNKALPAPIEKTISKAKGQPREPQVYTLPP SRDELTKNQVSLWCLVKGFYPSDIAVEWESNGQPENNYKTTPPVLD SDGSFFLYSKLTVDKSRWQQGNVFSCSVMHEALHNHYTQKSLSLSP GK |
| 37 | hFc-hole | ASKTHTCPPCPAPELLGGPSVFLFPPKPKDTLMISRTPEVTCVVVD VSHEDPEVKFNWYVDGVEVHNAKTKPREEQYNSTYRVVSVLTVLHQ DWLNGKEYKCKVSNKALPAPIEKTISKAKGQPREPQVYTLPPSRDE LTKNQVSLSCAVKGFYPSDIAVEWESNGQPENNYKTTPPVLDSDGS FFLVSKLTVDKSRWQQGNVFSCSVMHEALHNHYTQKSLSLSPGK |

### 7.6.2 Construction of Bispecific Antibodies With TCR Constant Domains

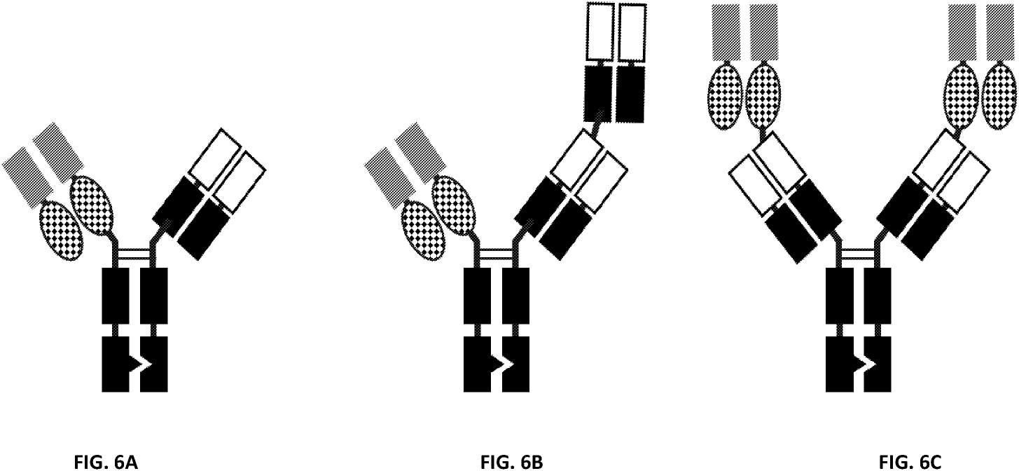

An alternative method of preventing the assembly of nonspecific antibodies with mismatched heavy and light chains is to replace one heavy chain CR1 constant domain with a T-cell receptor alpha (TCRα) constant domain and replace the corresponding LC constant domain with a TCRβ constant domain, as shown in FIGS. 6A-6C and described by Xu in WO 2019/057122. αCD38(TCR) x αICAM1 is a bispecific antibody with a CD38 binding domain derived from h18E4-19 linked to TCR sequences, an ICAM1 binding domain and associated HC CR1 and LC constant domains derived from h11F2-6, and an IgG1 Fc domain with knob-into-hole mutations to facilitate assembly of bispecific antibodies while inhibiting assembly of monospecific anti-CD38 or monospecific anti-ICAM1 antibodies. Sequences of the heavy and light chains of αCD38(TCR) x αICAM1 and its component parts are presented in Table 13. The structures of αCD38(scFv) x αICAM1 and αCD38(TCR) x αICAM1 are compared in FIGS. 7A-7B. CDR domain sequences shared by αCD38(scFv) x αICAM1 and αCD38(TCR) x αICAM1 are presented in Table 14.

TABLE 13

Sequences of the component parts of αCD38(TCR) x αICAM1

| Polypeptide | Sequence | SEQ ID NO |
|---|---|---|
| αCD38 h18E4-19 HC | EVQLLESGGGLVQPGGSLRLSCAASGFSFNNYWIIWIRQAPGKGLEWVAAIYSPSGD IKYYADWAKGRFTVSKDNSKNTVYLQMNSLRAEDTAVYYCARELSGSSYEGYFESWG QGTLVTVLEDLKNVFPPEVAVFEPSEAEISHTQKATLVCLATGFYPDHVELSWWVNG KEVHSGVCTDPQPLKEQPALQDSRYALSSRLRVSATFWQNPRNHFRCQVQFYGLSEN DEWTQDRAKPVTQIVSAEAWGRASDKTHTCPPCPAPELLGGPSVFLFPPKPKDTLMI SRTPEVTCVVVDVSHEDPEVKFNWYVDGVEVHNAKTKPREEQYNSTYRVVSVLTVLH QDWLNGKEYKCKVSNKALPAPIEKTISKAKGQPREPQVCTLPPSREEMTKNQVSLSC AVKGFYPSDIAVEWESNGQPENNYKTTPPVLDSDGSFFLVSKLTVDKSRWQQGNVFS CSVMHEALHNHYTQKSLSLSPG | 38 |
| αCD38 h18E4-19 LC | DIQLTQSPSTLSASVGDRVTITCRSSQSVYGHNWLAWYQQKPGKAPKLLMYRASNLA SGVPSRFSGSGSGTEFTLTISSVQPDDFATYYCQGYYSGGSYAFGGGTKVEIKPDIQ NPDPAVYQLRDSKSSDKSVCLFTDFDSQTQVSQSKDSDVYITDKCVLDMRSMDFKSN SAVAWSQKSDFACANAFQNSIIPEDTFFPSPESS | 39 |
| αCD38 h18E4-19 HCV | EVQLLESGGGLVQPGGSLRLSCAASGFSFNNYWIIWIRQAPGKGLEWVAAIYSPSGD IKYYADWAKGRFTVSKDNSKNTVYLQMNSLRAEDTAVYYCARELSGSSYEGYFESWG QGTLVTV | 40 |
| αCD38 h18E4-19 LCV | DIQLTQSPSTLSASVGDRVTITCRSSQSVYGHNWLAWYQQKPGKAPKLLMYRASNLA SGVPSRFSGSGSGTEFTLTISSVQPDDFATYYCQGYYSGGSYAFGGGTKVEIK | 41 |
| αICAM1 h11F2-6 HC | EVQVLESGGGLVQPGGSLRLSCAVSGFSLSSHAMGWVRQAPGKGLEWIGIIGSSDRT YYASWAKGRFTISKDNSKNTVYLQINSPRAEDTAVYYCVRDPYDSYDEGYRLWGQGT LVTVSSASTKGPSVFPLAPSSKSTSGGTAALGCLVKDYFPEPVTVSWNSGALTSGVH TFPAVLQSSGLYSLSSVVTVPSSSLGTQTYICNVNHKPSNTKVDKRVEPKSCDKTHT CPPCPAPELLGGPSVFLFPPKPKDTLMISRTPEVTCVVVDVSHEDPEVKFNWYVDGV EVHNAKTKPREEQYNSTYRVVSVLTVLHQDWLNGKEYKCKVSNKALPAPIEKTISKA KGQPREPQVYTLPPCREEMTKNQVSLWCLVKGFYPSDIAVEWESNGQPENNYKTTPP VLDSDGSFFLYSKLTVDKSRWQQGNVFSCSVMHEALHNHYTQKSLSLSPG | 42 |
| αICAM1 h11F2-6 LC | DIQMTQSPSTLSASVGDRVTITCQASQSIYRYLSWYQQKPGKAPKFLIYDASKLASG VPSRFSGSGSGTEFTLTISSVQPDDFATYYCQQAYSSGSIDNAFGGGTKVEIKRTVA APSVFIFPPSDEQLKSGTASVVCLLNNFYPREAKVQWKVDNALQSGNSQESVTEQDS KDSTYSLSSTLTLSKADYEKHKVYACEVTHQGLSSPVTKSFNRGEC | 43 |
| αICAM1 h11F2-6 HCV | EVQVLESGGGLVQPGGSLRLSCAVSGFSLSSHAMGWVRQAPGKGLEWIGIIGSSDRT YYASWAKGRFTISKDNSKNTVYLQINSPRAEDTAVYYCVRDPYDSYDEGYRLWGQGT LVTVSS | 44 |
| αICAM1 h11F2-6 LCV | DIQMTQSPSTLSASVGDRVTITCQASQSIYRYLSWYQQKPGKAPKFLIYDASKLASG VPSRFSGSGSGTEFTLTISSVQPDDFATYYCQQAYSSGSIDNAFGGGTKVEIK | 45 |
| TCRα | PDIQNPDPAVYQLRDSKSSDKSVCLFTDFDSQTQVSQSKDSDVYITDKCVLD MRSMDFKSNSAVAWSQKSDFACANAFQNSIIPEDTFFPSPESS | 46 |
| TCRβ | LEDLKNVFPPEVAVFEPSEAEISHTQKATLVCLATGFYPDHVELSWWVNGKE VHSGVCTDPQPLKEQPALQDSRYALSSRLRVSATFWQNPRNHFRCQVQFYGL SENDEWTQDRAKPVTQIVSAEAWGRAS | 47 |
| Fc-knob | EPKSCDKTHTCPPCPAPELLGGPSVFLFPPKPKDTLMISRTPEVTCVVVDVSHEDPE VKFNWYVDGVEVHNAKTKPREEQYNSTYRVVSVLTVLHQDWLNGKEYKCKVSNKALP APIEKTISKAKGQPREPQVYTLPPCREEMTKNQVSLWCLVKGFYPSDIAVEWESNGQ PENNYKTTPPVLDSDGSFFLYSKLTVDKSRWQQGNVFSCSVMHEALHNHYTQKSLSL SPG | 48 |
| Fc-hole | DKTHTCPPCPAPELLGGPSVFLFPPKPKDTLMISRTPEVTCVVVDVSHEDPEVKFNW YVDGVEVHNAKTKPREEQYNSTYRVVSVLTVLHQDWLNGKEYKCKVSNKALPAPIEK TISKAKGQPREPQVCTLPPSREEMTKNQVSLSCAVKGFYPSDIAVEWESNGQPENNY KTTPPVLDSDGSFFLVSKLTVDKSRWQQGNVFSCSVMHEALHNHYTQKSLSLSPG | 49 |

TABLE 14

CDR domain sequences of humanized bispecific antibodies αCD38(scFv) x αICAM1 and αCD38(TCR) x αICAM1

| Antigen | Domain | Sequence¹ | SEQ ID NO |
|---|---|---|---|
| CD38 | HC CDR1 | NYWII | 50 |
| | HC CDR2 | AIYSPSGDIKYYADWAKG | 51 |
| | HC CDR3 | ELSGSSYEGYFES | 52 |
| | LC CDR1 | RSSQSVYGHNWLA | 53 |
| | LC CDR2 | RASNLAS | 54 |
| | LC CDR3 | QGYYSGGSYA | 55 |
| ICAM1 | HC CDR1 | SHAMG | 56 |
| | HC CDR2 | IIGSSDRTYYASWAKG | 57 |
| | HC CDR3 | DPYDSYDEGYRL | 58 |
| | LC CDR1 | QASQSIYRYLS | 59 |
| | LC CDR2 | DASKLAS | 60 |
| | LC CDR3 | QQAYSSGSIDNA | 61 |

¹SEQ ID NO: 52 is the same amino acid sequence as SEQ ID NO: 9; SEQ ID NO: 54 is the same amino acid sequence as SEQ ID NO: 11; SEQ ID NO: 56 is the same amino acid sequence as SEQ ID NO: 17; SEQ ID NO: 57 is the same amino acid sequence as SEQ ID NOs: 18 and 28; SEQ ID NO: 59 is the same amino acid sequence as SEQ ID NO: 20; SEQ ID NO: 60 is the same amino acid sequence as SEQ ID NO: 21; SEQ ID NO: 61 is the same amino acid sequence as SEQ ID NO: 22.

### 7.6.3 Thermostability of Humanized Bispecific Antibodies

Thermostability was measured by differential scanning calorimetry (DSC) on an automated MicroCal VP-Capillary DSC equipped with a 96-well plate autosampler. Bispecific antibodies were diluted to 1 mg/mL in 20 mM histidine, 150 mM NaCl, pH 5.5 buffer. 400 µL of diluted antibody was analyzed per well of a 96-well plate. The bispecific antibody samples were heated from 25° C. to 100° C. at a rate of 60° C./h. Melting temperatures (Tₘ) were calculated according to a non-2-state model using Origin 7.0 software, with subtraction of background from a protein-free reference sample and normalization to protein concentration.

The αCD38(scFv) x αICAM1 and αCD38(TCR) x αICAM1 bispecific antibodies each had three melting temperatures. The lowest thermal transition, which might represent unfolding of the CD38-binding domain, was higher for αCD38(TCR) x αICAM1 (65° C.) than αCD38(scFv) x αICAM1 (60° C.).

### 7.6.4 Binding Activity of Humanized Bispecific Antibodies

The binding affinities αCD38(scFv) x αICAM1 and αCD38(TCR) x αICAM1 were determined by surface plasmon resonance. As expected, the affinities of these bispecific antibodies for hCD38 (KD: 2 - 5 nM) and hICAM1 (KD: 0.1 - 0.15 nM) were similar to each other.

### 7.6.5 ADCC Activities of Bispecific and Afucosylated Antibodies

αCD38(scFv) x αICAM1 and αCD38(TCR) x αICAM1 had potent ADCC activity against cell lines derived from a wide variety of tumors and having widely varying CD38 and ICAM1 expression levels, as shown in Table 15. The bispecific antibodies had higher ADCC activity than bivalent CD38 antibodies with the same anti-CD38 CDRs in cell lines with relatively low CD38 expression. Likewise, the bispecific antibodies had higher ADCC activity than bivalent ICAM1 antibodies with the same anti-ICAM1 CDRs in cells with low to medium ICAM1 expression.

When antibodies were produced in a CHO cell line lacking FUT8 (fucosyltransferase 8) expression, afucosylation enhanced ADCC activity of all bispecific and monospecific antibodies tested and was effective on all CD38+ ICAM1+ tumor cell lines tested, as shown in Table 15.

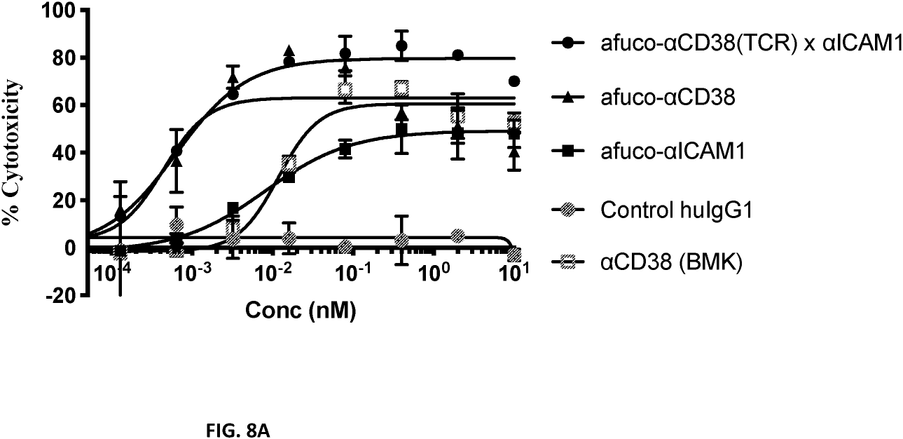

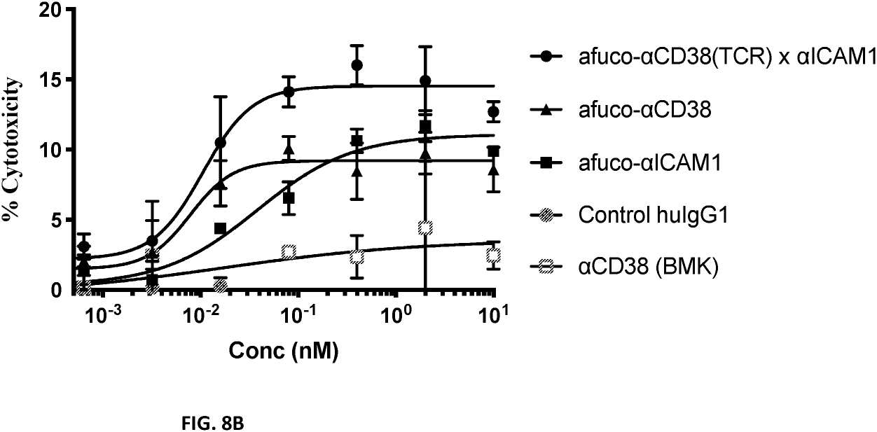

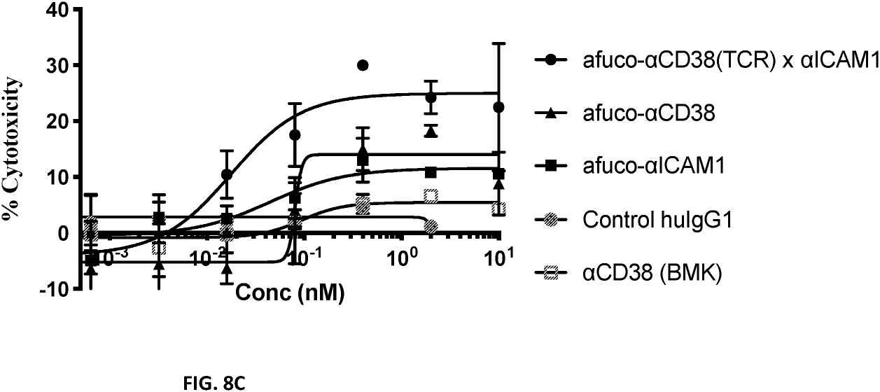

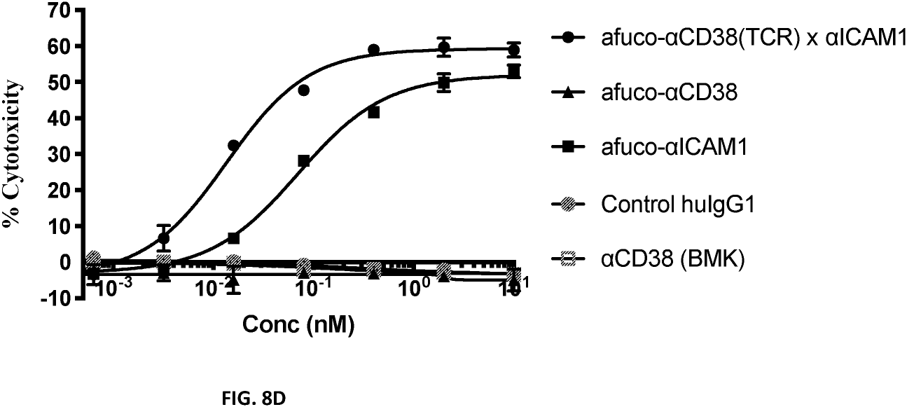

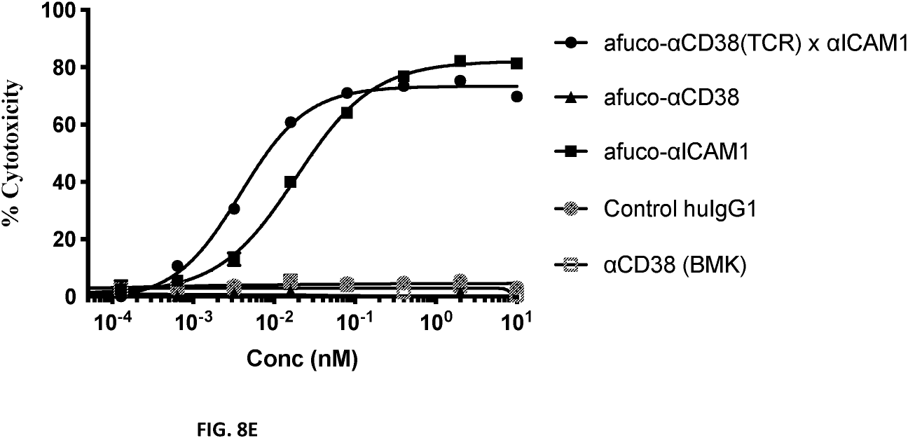

FIGS. 8A-8E compare the ADCC activity of afucosylated αCD38(TCR) x αICAM1 with afucosylated bivalent anti-CD38 or anti-ICAM antibodies with the same binding domains and the anti-CD38 BMK benchmark antibody. Afuco-αCD38(TCR) x αICAM1 showed superior ADCC activity in all tumor cell lines tested, including tumor cell lines with low CD38 or ICAM1 surface expression. Collectively, these ADCC assays demonstrate that humanized bispecific CD38 x ICAM1 antibodies have more versatile ADCC activity against a wider range of transformed cells than monospecific αCD38 or monospecific αICAM1 antibodies and that this activity is further enhanced by afucosylation.

TABLE 15

ADCC activity (EC50 in pM) of bispecific and monospecific antibodies on the indicated cell lines. The antibodies were produced in standard CHO cells or in fucosyltransferase-deficient CHO cells

| | Antibody | Daudi | Raji | HuNS1 | NCI-H2342 | NCI-H2291 |
|---|---|---|---|---|---|---|
| Producer cell | Target cell cancer | Lymphoma | Lymphoma | Myeloma | Lung | Lung |
| CHO FUT8 -/ | αCD38(TCR) × αICAM1 | 0.6 | 11 | 20 | 10 | 4 |
| | αCD38(scFv) × αICAM1 | 0.7 | 4 | 10 | 10 | 2 |
| | αCD38 | 0.4 | 8 | 80 | n.d. | n.d. |
| | αICAM1 | 8.0 | 38 | 40 | 70 | 19 |
| CHO | αCD38(TCR) × αICAM1 | 47.9 | 51 | 100 | 60 | 71 |
| | αCD38(scFv) × αICAM1 | 20.1 | 67 | 40 | 30 | 32 |
| | αCD38 | 12.7 | 14 | 80 | n.d. | n.d. |
| | αICAM1 | n.d. | n.d. | n.d. | 690 | 329 |
| CHO | αCD38(BMK) | 11.7 | 18 | 90 | n.d. | n.d. |
| | IgG1 | n.d. | n.d. | n.d. | n.d. | n.d. |

*n.d. not detected

### 7.7 Example 5: In Vivo Xenograft Tumor Models

Mice with cell-derived xenografts (CDX) or patient-derived xenografts (PDX) were used to test whether humanized CD38 x ICAM1 bispecific antibodies possess the ability to inhibit tumor growth. Conventional or afucosylated versions of the humanized αCD38(scFv) x αICAM1 and αCD38(TCR) x αICAM1 bispecific antibodies were compared to monospecific ICAM1 and CD38 antibodies in Raji (lymphoma), KMS-26 (multiple myeloma), and HCC44 (lung cancer) CDX models, and a LY3071 (lymphoma) PDX model.

### 7.7.1 In Vivo Activities in a Subcutaneous Lymphoma (Raji) CDX Model

In vivo anti-tumor activities of the CD38/ICAM1 bispecific antibodies were investigated in a Raji Lymphoma CDX model. The Raji tumor cells were maintained in vitro as a monolayer culture in RPMI-1640 medium supplemented with 10 % heat-inactivated fetal bovine serum, 100 U/mL penicillin, and 100 µg/mL streptomycin at 37° C. in an atmosphere of 5% CO₂ in air. The tumor cells were routinely sub-cultured twice weekly by trypsin-EDTA treatment. The cells growing in an exponential growth phase were harvested and counted for tumor inoculation. Female CB.17 SCID mice (Beijing Charles River Laboratories) were inoculated subcutaneously with 10⁷ cells in 0.2 mL of PBS supplemented with BD Matrigel (1:1) for tumor development. Treatments were started on day 9 after tumor inoculation when the average tumor size reached approximately 181 mm³. The animals were assigned into groups using an Excel-based randomization software performing stratified randomization based upon their tumor volumes. Each group consisted of 5 tumor-bearing mice. The testing antibodies were administrated to the mice intravenously at a dose of 10 mg/kg, twice per week, in three successive weeks. Tumor volume was monitored, and animals were sacrificed if tumors reached 3000 mm³ in size or became necrotic.

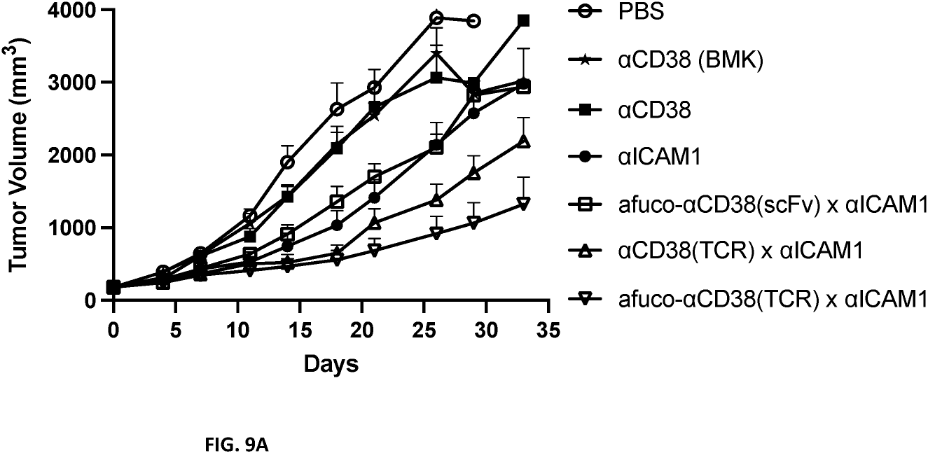

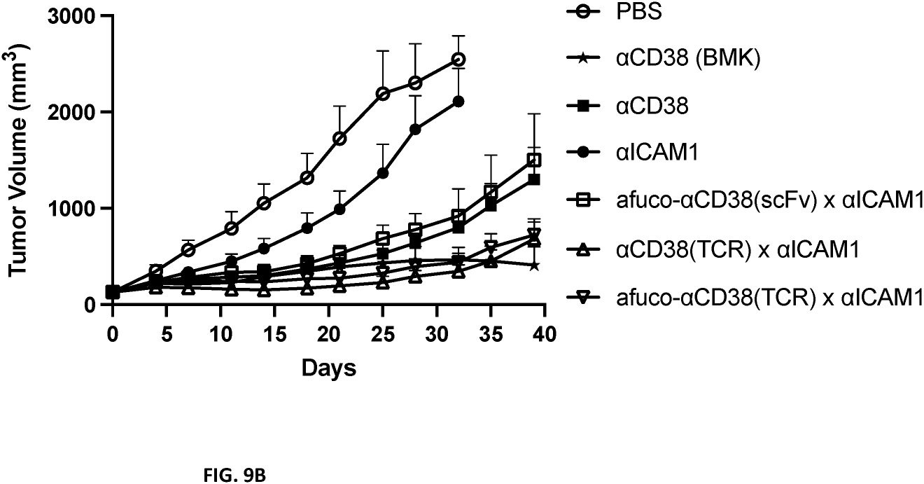

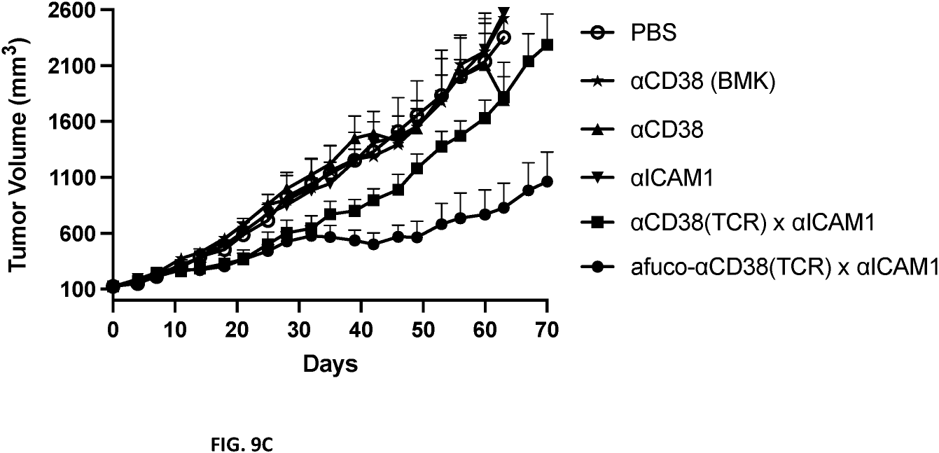

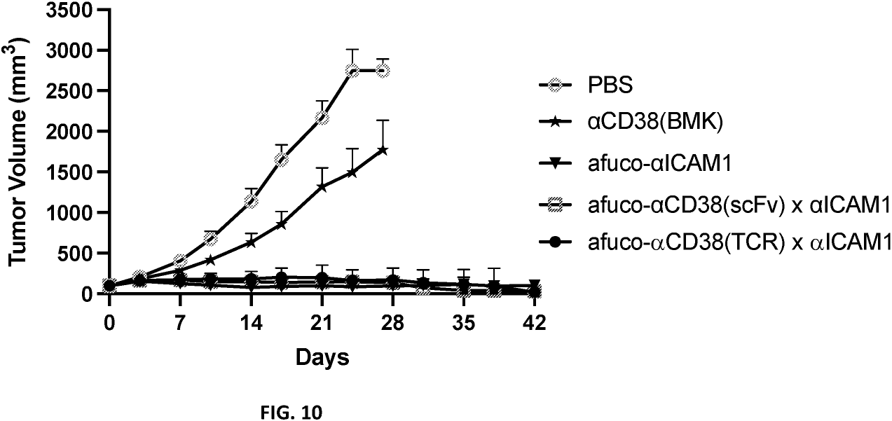

Untreated Raji cell tumors reached 4000 mm³ after 25 days (FIG. 9A). The αCD38(scFv) x αICAM1 and αCD38(TCR) x αICAM1 humanized bispecific antibodies both provided significant growth inhibition. The TCR format bispecific antibodies had greater activity than the scFv format bispecific antibodies. Both bispecific antibodies inhibited growth to a greater extent than a bivalent anti-CD38 antibody with the same binding domain and the benchmark bivalent anti-CD38 antibody. The bivalent anti-ICAM1 antibody inhibited Raji cell tumor growth as well as the αCD38(scFv) x αICAM1 bispecific antibody. The relatively high activity of the anti-ICAM1 antibody is consistent with high ICAM1 expression in Raji cells. Afuco-αCD38(TCR) x αICAM1 had the highest Raji cell tumor growth inhibition of all antibodies tested.

### 7.7.2 In Vivo Activities in a Subcutaneous Multiple Myeloma KMS-26 CDX Model

In vivo anti-tumor activities of the CD38xICAM1 bispecific antibodies were further investigated in a KMS-26 multiple myeloma CDX model. The KMS-26 tumor cells were maintained in vitro as a monolayer culture in RPMI 1640 medium supplemented with 10% heat-inactivated fetal bovine serum, 100 U/mL penicillin, and 100 µg/mL streptomycin, at 37° C. in an atmosphere of 5% CO2 in air. The tumor cells were routinely sub-cultured twice weekly by trypsin-EDTA treatment. The cells growing in an exponential growth phase were harvested and counted for tumor inoculation.

Female CB.17 SCID mice (Beijing Charles River Laboratories) were inoculated subcutaneously with 5 × 10⁶ cells in 0.2 mL of PBS supplemented with BD Matrigel (1:1) for tumor development. Treatments were started on day 10 after tumor inoculation when the average tumor size reached approximately 130 mm³. The animals were assigned into groups using an Excel-based randomization software performing stratified randomization based upon their tumor volumes. Each group consisted of 5 tumor-bearing mice. The testing antibodies were administrated to the mice intravenously at a dose of 10 mg/kg, twice per week, in three successive weeks. Tumor volume was monitored, and animals were sacrificed if tumors reached 3000 mm³ in size or became necrotic.

Untreated KMS-26 cell tumors reached 2500 mm³ after 30 days (FIG. 9B). The αCD38(scFv) x αICAM1 and αCD38(TCR) x αICAM1 humanized bispecific antibodies both provided significant growth inhibition. The TCR format bispecific antibodies had greater activity than the scFv format bispecific antibodies. Both bispecific antibodies inhibited growth to a greater extent than a bivalent anti-ICAM1 antibody with the same binding domain. Bivalent anti-CD38 antibodies had relatively high growth inhibition activity against KMS-26 cell tumors. Afuco-αCD38(TCR) x αICAM1 had the highest KMS-26 cell tumor growth inhibition of all antibodies tested.

### 7.7.3 In Vivo Activities in a Subcutaneous Lung Cancer (HCC44) CDX Model

In vivo anti-tumor activities of the CD38/ICAM1 Bispecifics were further investigated in a lung cancer CDX model (HCC44). The HCC44 tumor cells were maintained in vitro as a monolayer culture in DMEM medium supplemented with 10% heat-inactivated fetal bovine serum, 100 U/mL penicillin and 100 µg/mL streptomycin, at 37° C. in an atmosphere of 5% CO₂ in air. The tumor cells were routinely sub-cultured twice weekly by trypsin-EDTA treatment. The cells growing in an exponential growth phase were harvested and counted for tumor inoculation.

Female Balb/c nude mice (Shanghai Lingchang Laboratory Animal Technology Co., LTD.) were inoculated subcutaneously at the right flank with 5×10⁶ cells in 0.2 mL of PBS supplemented with BD Matrigel (1:1) for tumor development. Treatments were started on day 20 after tumor inoculation when the average tumor size reached approximately 120 mm³. The animals were assigned into groups using an Excel-based randomization software performing stratified randomization based upon their tumor volumes. Each group consisted of 5 tumor-bearing mice. The testing antibodies were administrated to the mice intravenously at a dose of 10 mg/kg, twice per week. Tumor volume was monitored, and animals were sacrificed if tumors reached 3000 mm³ in size or became necrotic.

Untreated HCC44 cell tumors reached 2300 mm³ after 60 days (FIG. 9C). Bivalent anti-CD38 and anti-ICAM1 did not inhibit the growth of HCC44 cell tumors whereas the αCD38(TCR) x αICAM1 humanized bispecific antibody showed significant growth inhibition. Afuco-αCD38(TCR) x αICAM1 had significantly higher HCC44 cell tumor growth inhibition activity compared to normally fucosylated αCD38(TCR) x αICAM1.

### 7.7.4 In Vivo Activities in a Subcutaneous Human Lymphoma (LY3071) PDX Model

Fresh tumor tissues from mice bearing established primary human cancer tissues were harvested and cut into small pieces (approximately 2-3 mm in diameter). Each mouse was inoculated subcutaneously at the right front flank with those tumor tissues for tumor development. The mice were randomized when the mean tumor size reaches approximately 80-120 mm³. 32 mice were randomly allocated to 4 study groups (8 mice per group). Randomization was performed using the “Matched distribution” method or the “Stratified” method (StudyDirector™ software, version 3.1.399.19). The testing antibodies were administrated to the mice intravenously at a dose of 10 mg/kg, twice per week, in three successive weeks. Tumor volume was monitored, and animals were sacrificed if tumors reached 3000 mm³ in size or became necrotic.

Untreated patient-derived tumors grew to >1600 mm³ in 22 days. Growth was partially inhibited by afucosylated anti-CD38 bivalent antibodies. The tumors shrunk when treated with afucosylated anti-ICAM1 bivalent, afucosylated αCD38(scFv) x αICAM1, and afucosylated αCD38(TCR) x αICAM1 humanized bispecific antibodies.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1-76. (canceled)

77. A bispecific antibody comprising one or more humanized CD38 binding domains, one or more humanized ICAM1 binding domains, and a human Fc domain.

78. The bispecific antibody of claim 77, wherein the humanized CD38 binding domain, the humanized ICAM1 binding domain, or both, comprises a single chain variable fragment (scFv).

79. The bispecific antibody of claim 77, wherein an isotype of the bispecific antibody is human IgG1.

80. The bispecific antibody of claim 77, wherein the human Fc domain is a heterodimeric Fc domain, wherein the heterodimeric Fc region comprises a knob chain and a hole chain, forming a knob-in-hole (KiH) structure.

81. The bispecific antibody of claim 80, wherein the knob chain comprises mutation corresponding to T366W and the hole chain comprises mutations corresponding to T366S, L368A, and Y407V, wherein amino acid position numbering is according to the EU index of Kabat et al.

82. The bispecific antibody of claim 80, wherein the knob chain comprises mutations corresponding to S354C, T366W and the hole chain comprises mutations corresponding to Y349C, T366S, L368A, and Y407V, wherein amino acid position numbering is according to the EU index of Kabat et al.

83. The bispecific antibody of claim 77, wherein the Fc domain is afucosylated.

84. The bispecific antibody of claim 77, wherein the Fc domain comprises one or more amino acid substitutions that enhance antigen-dependent cellular cytotoxicity (ADCC) activity.

85. The bispecific antibody of claim 84, wherein the Fc domain comprises mutations corresponding to S239D, 1332E, and A330L amino acid substitutions, and wherein the amino acid numbering is according to the EU index in Kabat et al.

86. The bispecific antibody of claim 77, wherein the humanized CD38 binding domain comprises the variable domain of an anti-CD38 IgG, and the ICAM1 binding domain comprises an anti-ICAM1 single chain variable fragment (anti-ICAM1 scFv).

87. The bispecific antibody of claim 77, wherein the humanized ICAM1 binding domain comprises the variable domain of an anti-ICAM1 IgG, and the CD38 binding domain comprises an anti-CD38 single chain variable fragment (anti-CD38 scFv).

88. The bispecific antibody of claim 77, further comprising a CH1 IgG domain and a CL IgG domain.

89. The bispecific antibody of claim 77, further comprising a T cell receptor (TCR) constant region, wherein the TCR constant region comprises a TCR alpha constant domain and a TCR beta constant domain.

90. The bispecific antibody of claim 77, wherein the humanized CD38 binding domain comprises a sequence at least 90% identical to SEQ ID NO: 40.

91. The bispecific antibody of claim 77, wherein the humanized CD38 binding domain comprises a sequence at least 90% identical to SEQ ID NO: 41.

92. The bispecific antibody of claim 77, wherein the humanized CD38 binding domain comprises a sequence at least 90% identical to SEQ ID NO: 35.

93. The bispecific antibody of claim 77, wherein the humanized CD38 binding domain comprises sequences at least 90% identical to SEQ ID NO: 50, SEQ ID NO: 51, SEQ ID NO: 52, SEQ ID NO: 53, SEQ ID NO: 54, and SEQ ID NO: 55.

94. The bispecific antibody of claim 77, wherein the humanized ICAM1 binding domain comprises a sequence at least 90% identical to SEQ ID NO: 44.

95. The bispecific antibody of claim 77, wherein the humanized ICAM1 binding domain comprises a sequence at least 90% identical to SEQ ID NO: 45.

96. The bispecific antibody of claim 77, wherein the humanized ICAM1 binding domain comprises sequences at least 90% identical to SEQ ID NO: 56, SEQ ID NO: 57, SEQ ID NO: 58, SEQ ID NO: 59, SEQ ID NO: 60, and SEQ ID NO: 61.

97. A method of treating a cancer in a subject comprising administering to the subject the bispecific antibody of claim 77, wherein the cancer comprises a cell that expresses CD38 and ICAM1 and, wherein the cancer comprises a solid tumor or a hematological malignancy.


SEQUENCE LISTING (120 sequences)

SEQ ID NO: 1 - protein, 274 aa, Homo sapiens
SGLNDIFEAQKIEWHEVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 2 - protein, 480 aa, Homo sapiens
QTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVNVLSPRYEGGSGGSGLNDIFEAQKIEWHEENLYFQ

SEQ ID NO: 3 - protein, 451 aa, Artificial Sequence
  Description of Artificial Sequence: Synthetic polypeptide
QSLEESGGDLVKPGASLTLTCTASGFSFNNYWICWIRQAPGKGLEWVACIYSPSGDIKYYANWAKGRFTVSKTSSTTVTLQMTSLTGADTATYFCARELSGSSYEGYFESWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 217 aa, Artificial Sequence
  Description of Artificial Sequence: Synthetic polypeptide
AQVLTQTPSSVSAAVGGTVTINCQCSQSVYGHNWLAWYQHKPGQPPKLLMYRASNLASGVPSRFKGSGSGSQFTLTIGEVQSDDAATYYCQGYYNGGSYAFGGGTEVVVRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 121 aa, Artificial Sequence
  Description of Artificial Sequence: Synthetic polypeptide
QSLEESGGDLVKPGASLTLTCTASGFSFNNYWICWIRQAPGKGLEWVACIYSPSGDIKYYANWAKGRFTVSKTSSTTVTLQMTSLTGADTATYFCARELSGSSYEGYFESWGPGTLVTVSS

SEQ ID NO: 6 - protein, 110 aa, Artificial Sequence
  Description of Artificial Sequence: Synthetic polypeptide
AQVLTQTPSSVSAAVGGTVTINCQCSQSVYGHNWLAWYQHKPGQPPKLLMYRASNLASGVPSRFKGSGSGSQFTLTIGEVQSDDAATYYCQGYYNGGSYAFGGGTEVVVR

SEQ ID NO: 7 - protein, 5 aa, Artificial Sequence
  Description of Artificial Sequence: Synthetic peptide
NYWIC

SEQ ID NO: 8 - protein, 18 aa, Artificial Sequence
  Description of Artificial Sequence: Synthetic peptide
CIYSPSGDIKYYANWAKG

SEQ ID NO: 9 - protein, 13 aa, Artificial Sequence
  Description of Artificial Sequence: Synthetic peptide
ELSGSSYEGYFES

SEQ ID NO: 10 - protein, 13 aa, Artificial Sequence
  Description of Artificial Sequence: Synthetic peptide
QCSQSVYGHNWLA

SEQ ID NO: 11 - protein, 7 aa, Artificial Sequence
  Description of Artificial Sequence: Synthetic peptide
RASNLAS

SEQ ID NO: 12 - protein, 10 aa, Artificial Sequence
  Description of Artificial Sequence: Synthetic peptide
QGYYNGGSYA

SEQ ID NO: 13 - protein, 447 aa, Artificial Sequence
  Description of Artificial Sequence: Synthetic polypeptide
QSVEESGGGLVKPGGTLTLTCTVSGFSLSSHAMGWVRQAPGKGLEYIGIIGSSDRTYYASWAKGRFTISKTSTTVDLKITSPTTEDTATYFCVRDPYDSYDDGYRLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 217 aa, Artificial Sequence
  Description of Artificial Sequence: Synthetic polypeptide
AYDMTQTPASVEVAVGGTVTIKCQASQSIYRYLSWYQKKPGQRPKFLIYDASKLASGVPSRFEGSGSGTEFTLTISGAQSDDAATYYCQQAYSSGSIDNAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 117 aa, Artificial Sequence
  Description of Artificial Sequence: Synthetic polypeptide
QSVEESGGGLVKPGGTLTLTCTVSGFSLSSHAMGWVRQAPGKGLEYIGIIGSSDRTYYASWAKGRFTISKTSTTVDLKITSPTTEDTATYFCVRDPYDSYDDGYRLWGPGTLVTVSS

SEQ ID NO: 16 - protein, 110 aa, Artificial Sequence
  Description of Artificial Sequence: Synthetic polypeptide
AYDMTQTPASVEVAVGGTVTIKCQASQSIYRYLSWYQKKPGQRPKFLIYDASKLASGVPSRFEGSGSGTEFTLTISGAQSDDAATYYCQQAYSSGSIDNAFGGGTEVVVK

SEQ ID NO: 17 - protein, 5 aa, Artificial Sequence
  Description of Artificial Sequence: Synthetic peptide
SHAMG

SEQ ID NO: 18 - protein, 16 aa, Artificial Sequence
  Description of Artificial Sequence: Synthetic peptide
IIGSSDRTYYASWAKG

SEQ ID NO: 19 - protein, 12 aa, Artificial Sequence
  Description of Artificial Sequence: Synthetic peptide
DPYDSYDDGYRL

SEQ ID NO: 20 - protein, 11 aa, Artificial Sequence
  Description of Artificial Sequence: Synthetic peptide
QASQSIYRYLS

SEQ ID NO: 21 - protein, 7 aa, Artificial Sequence
  Description of Artificial Sequence: Synthetic peptide
DASKLAS

SEQ ID NO: 22 - protein, 12 aa, Artificial Sequence
  Description of Artificial Sequence: Synthetic peptide
QQAYSSGSIDNA

SEQ ID NO: 23 - protein, 447 aa, Artificial Sequence
  Description of Artificial Sequence: Synthetic polypeptide
QSVEESGGGLVKPGGTLTLTCTVSGFSLSTHAMGWVRQAPGKGLEYIGIIGSSDRTYYASWAKGRFTISKTSTTVDLKITSPTTEDTATYFCVRDPYDSFDDGYRLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 217 aa, Artificial Sequence
  Description of Artificial Sequence: Synthetic polypeptide
AYDMTQTPASVEVAVGGTVTIKCQASQSIYSYLSWYQQKPGQRPKFLIYDASKVASGVPSRFKGSGSGTEFTLTISAVQSDDAATYYCQQAYSSSNVDNAFGGGTEVMVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 117 aa, Artificial Sequence
  Description of Artificial Sequence: Synthetic polypeptide
QSVEESGGGLVKPGGTLTLTCTVSGFSLSTHAMGWVRQAPGKGLEYIGIIGSSDRTYYASWAKGRFTISKTSTTVDLKITSPTTEDTATYFCVRDPYDSFDDGYRLWGPGTLVTVSS

SEQ ID NO: 26 - protein, 110 aa, Artificial Sequence
  Description of Artificial Sequence: Synthetic polypeptide
AYDMTQTPASVEVAVGGTVTIKCQASQSIYSYLSWYQQKPGQRPKFLIYDASKVASGVPSRFKGSGSGTEFTLTISAVQSDDAATYYCQQAYSSSNVDNAFGGGTEVMVK

SEQ ID NO: 27 - protein, 5 aa, Artificial Sequence
  Description of Artificial Sequence: Synthetic peptide
THAMG

SEQ ID NO: 28 - protein, 16 aa, Artificial Sequence
  Description of Artificial Sequence: Synthetic peptide
IIGSSDRTYYASWAKG

SEQ ID NO: 29 - protein, 12 aa, Artificial Sequence
  Description of Artificial Sequence: Synthetic peptide
DPYDSFDDGYRL

SEQ ID NO: 30 - protein, 11 aa, Artificial Sequence
  Description of Artificial Sequence: Synthetic peptide
QASQSIYSYLS

SEQ ID NO: 31 - protein, 7 aa, Artificial Sequence
  Description of Artificial Sequence: Synthetic peptide
DASKVAS

SEQ ID NO: 32 - protein, 12 aa, Artificial Sequence
  Description of Artificial Sequence: Synthetic peptide
QQAYSSSNVDNA

SEQ ID NO: 33 - protein, 450 aa, Artificial Sequence
  Description of Artificial Sequence: Synthetic polypeptide
EVQVLESGGGLVQPGGSLRLSCAVSGFSLSSHAMGWVRQAPGKGLEWIGIIGSSDRTYYASWAKGRFTISKDNSKNTVYLQINSPRAEDTAVYYCVRDPYDSYDEGYRLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 217 aa, Artificial Sequence
  Description of Artificial Sequence: Synthetic polypeptide
DIQMTQSPSTLSASVGDRVTITCQASQSIYRYLSWYQQKPGKAPKFLIYDASKLASGVPSRFSGSGSGTEFTLTISSVQPDDFATYYCQQAYSSGSIDNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 484 aa, Artificial Sequence
  Description of Artificial Sequence: Synthetic polypeptide
DIQLTQSPSTLSASVGDRVTITCRSSQSVYGHNWLAWYQQKPGKAPKLLMYRASNLASGVPSRFSGSGSGTEFTLTISSVQPDDFATYYCQGYYSGGSYAFGGGTKVEIKGGGGSGGGGSGGGGSEVQLLESGGGLVQPGGSLRLSCAASGFSFNNYWIIWIRQAPGKGLEWVAAIYSPSGDIKYYADWAKGRFTVSKDNSKNTVYLQMNSLRAEDTAVYYCARELSGSSYEGYFESWGQGTLVTVSSGGSGGSGGASKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 36 - protein, 232 aa, Artificial Sequence
  Description of Artificial Sequence: Synthetic polypeptide
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 228 aa, Artificial Sequence
  Description of Artificial Sequence: Synthetic polypeptide
ASKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 38 - protein, 478 aa, Artificial Sequence
  Description of Artificial Sequence: Synthetic polypeptide
EVQLLESGGGLVQPGGSLRLSCAASGFSFNNYWIIWIRQAPGKGLEWVAAIYSPSGDIKYYADWAKGRFTVSKDNSKNTVYLQMNSLRAEDTAVYYCARELSGSSYEGYFESWGQGTLVTVLEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 39 - protein, 205 aa, Artificial Sequence
  Description of Artificial Sequence: Synthetic polypeptide
DIQLTQSPSTLSASVGDRVTITCRSSQSVYGHNWLAWYQQKPGKAPKLLMYRASNLASGVPSRFSGSGSGTEFTLTISSVQPDDFATYYCQGYYSGGSYAFGGGTKVEIKPDIQNPDPAVYQLRDSKSSDKSVCLFTDFDSQTQVSQSKDSDVYITDKCVLDMRSMDFKSNSAVAWSQKSDFACANAFQNSIIPEDTFFPSPESS

SEQ ID NO: 40 - protein, 121 aa, Artificial Sequence
  Description of Artificial Sequence: Synthetic polypeptide
EVQLLESGGGLVQPGGSLRLSCAASGFSFNNYWIIWIRQAPGKGLEWVAAIYSPSGDIKYYADWAKGRFTVSKDNSKNTVYLQMNSLRAEDTAVYYCARELSGSSYEGYFESWGQGTLVTV

SEQ ID NO: 41 - protein, 110 aa, Artificial Sequence
  Description of Artificial Sequence: Synthetic polypeptide
DIQLTQSPSTLSASVGDRVTITCRSSQSVYGHNWLAWYQQKPGKAPKLLMYRASNLASGVPSRFSGSGSGTEFTLTISSVQPDDFATYYCQGYYSGGSYAFGGGTKVEIK

SEQ ID NO: 42 - protein, 449 aa, Artificial Sequence
  Description of Artificial Sequence: Synthetic polypeptide
EVQVLESGGGLVQPGGSLRLSCAVSGFSLSSHAMGWVRQAPGKGLEWIGIIGSSDRTYYASWAKGRFTISKDNSKNTVYLQINSPRAEDTAVYYCVRDPYDSYDEGYRLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 43 - protein, 217 aa, Artificial Sequence; identical to SEQ ID NO: 34
  Description of Artificial Sequence: Synthetic polypeptide

SEQ ID NO: 44 - protein, 120 aa, Artificial Sequence
  Description of Artificial Sequence: Synthetic polypeptide
EVQVLESGGGLVQPGGSLRLSCAVSGFSLSSHAMGWVRQAPGKGLEWIGIIGSSDRTYYASWAKGRFTISKDNSKNTVYLQINSPRAEDTAVYYCVRDPYDSYDEGYRLWGQGTLVTVSS

SEQ ID NO: 45 - protein, 110 aa, Artificial Sequence
  Description of Artificial Sequence: Synthetic polypeptide
DIQMTQSPSTLSASVGDRVTITCQASQSIYRYLSWYQQKPGKAPKFLIYDASKLASGVPSRFSGSGSGTEFTLTISSVQPDDFATYYCQQAYSSGSIDNAFGGGTKVEIK

SEQ ID NO: 46 - protein, 95 aa, Artificial Sequence
  Description of Artificial Sequence: Synthetic polypeptide
PDIQNPDPAVYQLRDSKSSDKSVCLFTDFDSQTQVSQSKDSDVYITDKCVLDMRSMDFKSNSAVAWSQKSDFACANAFQNSIIPEDTFFPSPESS

SEQ ID NO: 47 - protein, 131 aa, Artificial Sequence
  Description of Artificial Sequence: Synthetic polypeptide
LEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRAS

SEQ ID NO: 48 - protein, 231 aa, Artificial Sequence
  Description of Artificial Sequence: Synthetic polypeptide
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 49 - protein, 226 aa, Artificial Sequence
  Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 50 - protein, 5 aa, Artificial Sequence
  Description of Artificial Sequence: Synthetic peptide
NYWII

SEQ ID NO: 51 - protein, 18 aa, Artificial Sequence
  Description of Artificial Sequence: Synthetic peptide
AIYSPSGDIKYYADWAKG

SEQ ID NO: 52 - protein, 13 aa, Artificial Sequence
  Description of Artificial Sequence: Synthetic peptide
ELSGSSYEGYFES

SEQ ID NO: 53 - protein, 13 aa, Artificial Sequence
  Description of Artificial Sequence: Synthetic peptide
RSSQSVYGHNWLA

SEQ ID NO: 54 - protein, 7 aa, Artificial Sequence
  Description of Artificial Sequence: Synthetic peptide
RASNLAS

SEQ ID NO: 55 - protein, 10 aa, Artificial Sequence
  Description of Artificial Sequence: Synthetic peptide
QGYYSGGSYA

SEQ ID NO: 56 - protein, 5 aa, Artificial Sequence
  Description of Artificial Sequence: Synthetic peptide
SHAMG

SEQ ID NO: 57 - protein, 16 aa, Artificial Sequence
  Description of Artificial Sequence: Synthetic peptide
IIGSSDRTYYASWAKG

SEQ ID NO: 58 - protein, 12 aa, Artificial Sequence
  Description of Artificial Sequence: Synthetic peptide
DPYDSYDEGYRL

SEQ ID NO: 59 - protein, 11 aa, Artificial Sequence
  Description of Artificial Sequence: Synthetic peptide
QASQSIYRYLS

SEQ ID NO: 60 - protein, 7 aa, Artificial Sequence
  Description of Artificial Sequence: Synthetic peptide
DASKLAS

SEQ ID NO: 61 - protein, 12 aa, Artificial Sequence
  Description of Artificial Sequence: Synthetic peptide
QQAYSSGSIDNA

SEQ ID NO: 62 - protein, 6 aa, Artificial Sequence
  Description of Artificial Sequence: Synthetic peptide
HHHHHH

SEQ ID NO: 63 - protein, 110 aa, Artificial Sequence
  Description of Artificial Sequence: Synthetic polypeptide
DIQMTQSPSTLSASVGDRVTITCRCSQSVYGHNWLAWYQQKPGKAPKLLIYRASNLASGVPSRFSGSGSGTEFTLTISSLQPDDFATYYCQGYYNGGSYAFGGGTKVEIK

SEQ ID NO: 64 - protein, 110 aa, Artificial Sequence
  Description of Artificial Sequence: Synthetic polypeptide
DIQLTQSPSTLSASVGDRVTITCRCSQSVYGHNWLAWYQQKPGKAPKLLIYRASNLASGVPSRFSGSGSGTEFTLTISSVQPDDFATYYCQGYYNGGSYAFGGGTKVEIK

SEQ ID NO: 65 - protein, 110 aa, Artificial Sequence
  Description of Artificial Sequence: Synthetic polypeptide
DIQLTQSPSTLSASVGDRVTITCRCSQSVYGHNWLAWYQQKPGKAPKLLMYRASNLASGVPSRFSGSGSGTEFTLTISSVQPDDFATYYCQGYYNGGSYAFGGGTKVEIK

SEQ ID NO: 66 - protein, 110 aa, Artificial Sequence
  Description of Artificial Sequence: Synthetic polypeptide
DIQLTQSPSTLSASVGDRVTITCRSSQSVYGHNWLAWYQQKPGKAPKLLIYRASNLASGVPSRFSGSGSGTEFTLTISSVQPDDFATYYCQGYYNGGSYAFGGGTKVEIK

SEQ ID NO: 67 - protein, 110 aa, Artificial Sequence
  Description of Artificial Sequence: Synthetic polypeptide
DIQLTQSPSTLSASVGDRVTITCRSSQSVYGHNWLAWYQQKPGKAPKLLMYRASNLASGVPSRFSGSGSGTEFTLTISSVQPDDFATYYCQGYYNGGSYAFGGGTKVEIK

SEQ ID NO: 68 - protein, 110 aa, Artificial Sequence
  Description of Artificial Sequence: Synthetic polypeptide
DIQLTQSPSTLSASVGDRVTITCRSSQSVYGHNWLAWYQQKPGKAPKLLMYRASNLASGVPSRFSGSGSGTEFTLTISSVQPDDFATYYCQGYYQGGSYAFGGGTKVEIK

SEQ ID NO: 69 - protein, 110 aa, Artificial Sequence
  Description of Artificial Sequence: Synthetic polypeptide
DIQLTQSPSTLSASVGDRVTITCRSSQSVYGHNWLAWYQQKPGKAPKLLMYRASNLASGVPSRFSGSGSGTEFTLTISSVQPDDAATYYCQGYYNGGSYAFGGGTKVEIK

SEQ ID NO: 70 - protein, 110 aa, Artificial Sequence
  Description of Artificial Sequence: Synthetic polypeptide
DIQLTQSPSTLSASVGDRVTITCRSSQSVYGHNWLAWYQQKPGKAPKLLMYRASNLASGVPSRFSGSGSGTEFTLTISSVQPDDFATYYCQGYYNAGSYAFGGGTKVEIK

SEQ ID NO: 71 - protein, 121 aa, Artificial Sequence; identical to SEQ ID NO: 5
  Description of Artificial Sequence: Synthetic polypeptide

SEQ ID NO: 72 - protein, 123 aa, Artificial Sequence
  Description of Artificial Sequence: Synthetic polypeptide
EVQLLESGGGLVQPGGSLRLSCAASGFSFNNYWICWVRQAPGKGLEWVSCIYSPSGDIKYYADSVKGRFTISRDNSKNTLYLQMNSLRAEDTAVYYCAKELSGSSYEGYFESWGQGTLVTVSS

SEQ ID NO: 73 - protein, 123 aa, Artificial Sequence
  Description of Artificial Sequence: Synthetic polypeptide
EVQLLESGGGLVQPGGSLRLSCAASGFSFNNYWICWIRQAPGKGLEWVACIYSPSGDIKYYADSVKGRFTVSKDNSKNTVYLQMNSLRAEDTAVYYCARELSGSSYEGYFESWGQGTLVTVSS

SEQ ID NO: 74 - protein, 123 aa, Artificial Sequence
  Description of Artificial Sequence: Synthetic polypeptide
EVQLLESGGGLVQPGGSLRLSCAASGFSFNNYWICWIRQAPGKGLEWVACIYSPSGDIKYYADWAKGRFTVSKDNSKNTVYLQMNSLRAEDTAVYYCARELSGSSYEGYFESWGQGTLVTVSS

SEQ ID NO: 75 - protein, 123 aa, Artificial Sequence
  Description of Artificial Sequence: Synthetic polypeptide
EVQLLESGGGLVQPGGSLRLSCAASGFSFNNYWIAWIRQAPGKGLEWVASIYSPSGDIKYYADWAKGRFTVSKDNSKNTVYLQMNSLRAEDTAVYYCARELSGSSYEGYFESWGQGTLVTVSS

SEQ ID NO: 76 - protein, 123 aa, Artificial Sequence
  Description of Artificial Sequence: Synthetic polypeptide
EVQLLESGGGLVQPGGSLRLSCAASGFSFNNYWISWIRQAPGKGLEWVAYIYSPSGDIKYYADWAKGRFTVSKDNSKNTVYLQMNSLRAEDTAVYYCARELSGSSYEGYFESWGQGTLVTVSS

SEQ ID NO: 77 - protein, 123 aa, Artificial Sequence
  Description of Artificial Sequence: Synthetic polypeptide
EVQLLESGGGLVQPGGSLRLSCAASGFSFNNYWIIWIRQAPGKGLEWVAAIYSPSGDIKYYADWAKGRFTVSKDNSKNTVYLQMNSLRAEDTAVYYCARELSGSSYEGYFESWGQGTLVTVSS

SEQ ID NO: 78 - protein, 110 aa, Artificial Sequence
  Description of Artificial Sequence: Synthetic polypeptide
DIQLTQSPSTLSASVGDRVTITCRSSQSVYGHNWLAWYQQKPGKAPKLLMYRASNLASGVPSRFSGSGSGTEFTLTISSVQPDDFATYYCAGYYSGGSYAFGGGTKVEIK

SEQ ID NO: 79 - protein, 110 aa, Artificial Sequence
  Description of Artificial Sequence: Synthetic polypeptide
DIQLTQSPSTLSASVGDRVTITCRSSQSVYGHNWLAWYQQKPGKAPKLLMYRASNLASGVPSRFSGSGSGTEFTLTISSVQPDDFATYYCQAYYSGGSYAFGGGTKVEIK

SEQ ID NO: 80 - protein, 110 aa, Artificial Sequence
  Description of Artificial Sequence: Synthetic polypeptide
DIQLTQSPSTLSASVGDRVTITCRSSQSVYGHNWLAWYQQKPGKAPKLLMYRASNLASGVPSRFSGSGSGTEFTLTISSVQPDDFATYYCQGAYSGGSYAFGGGTKVEIK

SEQ ID NO: 81 - protein, 110 aa, Artificial Sequence
  Description of Artificial Sequence: Synthetic polypeptide
DIQLTQSPSTLSASVGDRVTITCRSSQSVYGHNWLAWYQQKPGKAPKLLMYRASNLASGVPSRFSGSGSGTEFTLTISSVQPDDFATYYCQGYASGGSYAFGGGTKVEIK

SEQ ID NO: 82 - protein, 110 aa, Artificial Sequence
  Description of Artificial Sequence: Synthetic polypeptide
DIQLTQSPSTLSASVGDRVTITCRSSQSVYGHNWLAWYQQKPGKAPKLLMYRASNLASGVPSRFSGSGSGTEFTLTISSVQPDDFATYYCQGYYAGGSYAFGGGTKVEIK

SEQ ID NO: 83 - protein, 110 aa, Artificial Sequence
  Description of Artificial Sequence: Synthetic polypeptide
DIQLTQSPSTLSASVGDRVTITCRSSQSVYGHNWLAWYQQKPGKAPKLLMYRASNLASGVPSRFSGSGSGTEFTLTISSVQPDDFATYYCQGYYSAGSYAFGGGTKVEIK

SEQ ID NO: 84 - protein, 110 aa, Artificial Sequence
  Description of Artificial Sequence: Synthetic polypeptide
DIQLTQSPSTLSASVGDRVTITCRSSQSVYGHNWLAWYQQKPGKAPKLLMYRASNLASGVPSRFSGSGSGTEFTLTISSVQPDDFATYYCQGYYSGASYAFGGGTKVEIK

SEQ ID NO: 85 - protein, 110 aa, Artificial Sequence
  Description of Artificial Sequence: Synthetic polypeptide
DIQLTQSPSTLSASVGDRVTITCRSSQSVYGHNWLAWYQQKPGKAPKLLMYRASNLASGVPSRFSGSGSGTEFTLTISSVQPDDFATYYCQGYYSGGAYAFGGGTKVEIK

SEQ ID NO: 86 - protein, 110 aa, Artificial Sequence
  Description of Artificial Sequence: Synthetic polypeptide
DIQLTQSPSTLSASVGDRVTITCRSSQSVYGHNWLAWYQQKPGKAPKLLMYRASNLASGVPSRFSGSGSGTEFTLTISSVQPDDFATYYCQGYYSGGSAAFGGGTKVEIK

SEQ ID NO: 87 - protein, 123 aa, Artificial Sequence
  Description of Artificial Sequence: Synthetic polypeptide
EVQLLESGGGLVQPGGSLRLSCAASGFSFNNYWIIWIRQAPGKGLEWVAAIYSPSGDIKYYADWAKGRFTVSKDNSKNTVYLQMNSLRAEDTAVYYCARALSGSSYEGYFESWGQGTLVTVSS

SEQ ID NO: 88 - protein, 123 aa, Artificial Sequence
  Description of Artificial Sequence: Synthetic polypeptide
EVQLLESGGGLVQPGGSLRLSCAASGFSFNNYWIIWIRQAPGKGLEWVAAIYSPSGDIKYYADWAKGRFTVSKDNSKNTVYLQMNSLRAEDTAVYYCAREASGSSYEGYFESWGQGTLVTVSS

SEQ ID NO: 89 - protein, 123 aa, Artificial Sequence
  Description of Artificial Sequence: Synthetic polypeptide
EVQLLESGGGLVQPGGSLRLSCAASGFSFNNYWIIWIRQAPGKGLEWVAAIYSPSGDIKYYADWAKGRFTVSKDNSKNTVYLQMNSLRAEDTAVYYCARELAGSSYEGYFESWGQGTLVTVSS

SEQ ID NO: 90 - protein, 123 aa, Artificial Sequence
  Description of Artificial Sequence: Synthetic polypeptide
EVQLLESGGGLVQPGGSLRLSCAASGFSFNNYWIIWIRQAPGKGLEWVAAIYSPSGDIKYYADWAKGRFTVSKDNSKNTVYLQMNSLRAEDTAVYYCARELSASSYEGYFESWGQGTLVTVSS

SEQ ID NO: 91 - protein, 123 aa, Artificial Sequence
  Description of Artificial Sequence: Synthetic polypeptide
EVQLLESGGGLVQPGGSLRLSCAASGFSFNNYWIIWIRQAPGKGLEWVAAIYSPSGDIKYYADWAKGRFTVSKDNSKNTVYLQMNSLRAEDTAVYYCARELSGASYEGYFESWGQGTLVTVSS

SEQ ID NO: 92 - protein, 123 aa, Artificial Sequence
  Description of Artificial Sequence: Synthetic polypeptide
EVQLLESGGGLVQPGGSLRLSCAASGFSFNNYWIIWIRQAPGKGLEWVAAIYSPSGDIKYYADWAKGRFTVSKDNSKNTVYLQMNSLRAEDTAVYYCARELSGSAYEGYFESWGQGTLVTVSS

SEQ ID NO: 93 - protein, 123 aa, Artificial Sequence
  Description of Artificial Sequence: Synthetic polypeptide
EVQLLESGGGLVQPGGSLRLSCAASGFSFNNYWIIWIRQAPGKGLEWVAAIYSPSGDIKYYADWAKGRFTVSKDNSKNTVYLQMNSLRAEDTAVYYCARELSGSSAEGYFESWGQGTLVTVSS

SEQ ID NO: 94 - protein, 123 aa, Artificial Sequence
  Description of Artificial Sequence: Synthetic polypeptide
EVQLLESGGGLVQPGGSLRLSCAASGFSFNNYWIIWIRQAPGKGLEWVAAIYSPSGDIKYYADWAKGRFTVSKDNSKNTVYLQMNSLRAEDTAVYYCARELSGSSYAGYFESWGQGTLVTVSS

SEQ ID NO: 95 - protein, 123 aa, Artificial Sequence
  Description of Artificial Sequence: Synthetic polypeptide
EVQLLESGGGLVQPGGSLRLSCAASGFSFNNYWIIWIRQAPGKGLEWVAAIYSPSGDIKYYADWAKGRFTVSKDNSKNTVYLQMNSLRAEDTAVYYCARELSGSSYEAYFESWGQGTLVTVSS

SEQ ID NO: 96 - protein, 123 aa, Artificial Sequence
  Description of Artificial Sequence: Synthetic polypeptide
EVQLLESGGGLVQPGGSLRLSCAASGFSFNNYWIIWIRQAPGKGLEWVAAIYSPSGDIKYYADWAKGRFTVSKDNSKNTVYLQMNSLRAEDTAVYYCARELSGSSYEGAFESWGQGTLVTVSS

SEQ ID NO: 97 - protein, 123 aa, Artificial Sequence
  Description of Artificial Sequence: Synthetic polypeptide
EVQLLESGGGLVQPGGSLRLSCAASGFSFNNYWIIWIRQAPGKGLEWVAAIYSPSGDIKYYADWAKGRFTVSKDNSKNTVYLQMNSLRAEDTAVYYCARELSGSSYEGYAESWGQGTLVTVSS

SEQ ID NO: 98 - protein, 123 aa, Artificial Sequence
  Description of Artificial Sequence: Synthetic polypeptide
EVQLLESGGGLVQPGGSLRLSCAASGFSFNNYWIIWIRQAPGKGLEWVAAIYSPSGDIKYYADWAKGRFTVSKDNSKNTVYLQMNSLRAEDTAVYYCARELSGSSYEGYFASWGQGTLVTVSS

SEQ ID NO: 99 - protein, 123 aa, Artificial Sequence
  Description of Artificial Sequence: Synthetic polypeptide
EVQLLESGGGLVQPGGSLRLSCAASGFSFNNYWIIWIRQAPGKGLEWVAAIYSPSGDIKYYADWAKGRFTVSKDNSKNTVYLQMNSLRAEDTAVYYCARELSGSSYEGYFEAWGQGTLVTVSS

SEQ ID NO: 100 - protein, 110 aa, Artificial Sequence
  Description of Artificial Sequence: Synthetic polypeptide
DIQMTQSPSTLSASVGDRVTITCRASQSIYSYLSWYQQKPGKAPKLLIYDASKVASGVPSRFSGSGSGTEFTLTISSLQPDDFATYYCQQAYSSSNVDNAFGGGTKVEIK

SEQ ID NO: 101 - protein, 110 aa, Artificial Sequence
  Description of Artificial Sequence: Synthetic polypeptide
DIQMTQSPSTLSASVGDRVTITCRASQSIYSYLSWYQQKPGKAPKFLIYDASKVASGVPSRFSGSGSGTEFTLTISSVQPDDFATYYCQQAYSSSNVDNAFGGGTKVEIK

SEQ ID NO: 102 - protein, 110 aa, Artificial Sequence
  Description of Artificial Sequence: Synthetic polypeptide
DYQMTQSPSTLSASVGDRVTITCRASQSIYSYLSWYQQKPGKAPKFLIYDASKVASGVPSRFSGSGSGTEFTLTISSVQPDDFATYYCQQAYSSSNVDNAFGGGTKVEIK

SEQ ID NO: 103 - protein, 110 aa, Artificial Sequence
  Description of Artificial Sequence: Synthetic polypeptide
DYQMTQSPSTLSASVGDRVTITCRASQSIYSYLSWYQQKPGKAPKFLIYDASKVASGVPSRFSGSGSGTEFTLTISSVQPDDAATYYCQQAYSSSNVDNAFGGGTKVEVK

SEQ ID NO: 104 - protein, 117 aa, Artificial Sequence; identical to SEQ ID NO: 25
  Description of Artificial Sequence: Synthetic polypeptide

SEQ ID NO: 105 - protein, 120 aa, Artificial Sequence
  Description of Artificial Sequence: Synthetic polypeptide
EVQLLESGGGLVQPGGSLRLSCAASGFSLSTHAMGWVRQAPGKGLEWVSIIGSSDRTYYADSVKGRFTISRDNSKNTLYLQMNSLRAEDTAVYYCAKDPYDSFDDGYRLWGQGTLVTVSS

SEQ ID NO: 106 - protein, 120 aa, Artificial Sequence
  Description of Artificial Sequence: Synthetic polypeptide
EVQVLESGGGLVQPGGSLRLSCAVSGFSLSTHAMGWVRQAPGKGLEWIGIIGSSDRTYYADSVKGRFTISKDNSKNTVYLQMNSLRAEDTAVYYCVRDPYDSFDDGYRLWGQGTLVTVSS

SEQ ID NO: 107 - protein, 120 aa, Artificial Sequence
  Description of Artificial Sequence: Synthetic polypeptide
EVQVLESGGGLVQPGGSLRLSCAVSGFSLSTHAMGWVRQAPGKGLEWIGIIGSSDRTYYADWAKGRFTISKDNSKNTVYLQMNSLRAEDTAVYYCVRDPYDSFDDGYRLWGQGTLVTVSS

SEQ ID NO: 108 - protein, 120 aa, Artificial Sequence
  Description of Artificial Sequence: Synthetic polypeptide
EVQVLESGGGLVQPGGSLRLSCAVSGFSLSTHAMGWVRQAPGKGLEWIGIIGSSDRTYYADWAKGRFTISKDNSKNTVYLQINSPRAEDTAVYYCVRDPYDSFDDGYRLWGQGTLVTVSS

SEQ ID NO: 109 - protein, 120 aa, Artificial Sequence
  Description of Artificial Sequence: Synthetic polypeptide
EVQVLESGGGLVQPGGSLRLSCAVSGFSLSTHAMGWVRQAPGKGLEYIGIIGSSDRTYYADWAKGRFTISKDNSKNTVYLQMNSLRAEDTAVYYCVRDPYDSFDDGYRLWGQGTLVTVSS

SEQ ID NO: 110 - protein, 120 aa, Artificial Sequence
  Description of Artificial Sequence: Synthetic polypeptide
EVQVLESGGGLVQPGGSLRLSCAVSGFSLSTHAMGWVRQAPGKGLEYIGIIGSSDRTYYADWAKGRFTISKDNSKNTVYLQINSPRAEDTAVYYCVRDPYDSFDDGYRLWGQGTLVTVSS

SEQ ID NO: 111 - protein, 120 aa, Artificial Sequence
  Description of Artificial Sequence: Synthetic polypeptide
EVQVLESGGGLVQPGGSLRLSCAVSGFSLSTHAMGWVRQAPGKGLEYIGIIGSSDRTYYADWAKGRFTISKTSTKNTVYLQMNSLRAEDTAVYYCVRDPYDSFDDGYRLWGQGTLVTVSS

SEQ ID NO: 112 - protein, 110 aa, Artificial Sequence
  Description of Artificial Sequence: Synthetic polypeptide
DYQMTQSPSTLSASVGDRVTITCQASQSIYRYLSWYQQKPGKAPKFLIYDASKLASGVPSRFSGSGSGTEFTLTISSVQPDDAATYYCQQAYSSGSIDNAFGGGTKVEVK

SEQ ID NO: 113 - protein, 110 aa, Artificial Sequence
  Description of Artificial Sequence: Synthetic polypeptide
DYQMTQSPSTLSASVGDRVTITCQASQSIYRYLSWYQQKPGKAPKFLIYDASKLASGVPSRFSGSGSGTEFTLTISSLQPDDAATYYCQQAYSSGSIDNAFGGGTKVEIK

SEQ ID NO: 114 - protein, 117 aa, Artificial Sequence; identical to SEQ ID NO: 15
  Description of Artificial Sequence: Synthetic polypeptide

SEQ ID NO: 115 - protein, 120 aa, Artificial Sequence
  Description of Artificial Sequence: Synthetic polypeptide
EVQVLESGGGLVQPGGSLRLSCAVSGFSLSSHAMGWVRQAPGKGLEYIGIIGSSDRTYYASWAKGRFTISKDNSKNTVYLQINSPRAEDTAVYYCVRDPYDSYDDGYRLWGQGTLVTVSS

SEQ ID NO: 116 - protein, 120 aa, Artificial Sequence
  Description of Artificial Sequence: Synthetic polypeptide
EVQVLESGGGLVQPGGSLRLSCAVSGFSLSSHAMGWVRQAPGKGLEYIGIIGSSDRTYYASWAKGRFTISKDNSKNTVYLQMNSLRAEDTAVYYCVRDPYDSYDDGYRLWGQGTLVTVSS

SEQ ID NO: 117 - protein, 120 aa, Artificial Sequence
  Description of Artificial Sequence: Synthetic polypeptide
EVQVLESGGGLVQPGGSLRLSCAVSGFSLSSHAMGWVRQAPGKGLEWIGIIGSSDRTYYASWAKGRFTISKDNSKNTVYLQINSPRAEDTAVYYCVRDPYDSYDDGYRLWGQGTLVTVSS

SEQ ID NO: 118 - protein, 120 aa, Artificial Sequence
  Description of Artificial Sequence: Synthetic polypeptide
EVQVLESGGGLVQPGGSLRLSCAVSGFSLSSHAMGWVRQAPGKGLEWIGIIGSSDRTYYASWAKGRFTISKDNSKNTVYLQINSPRAEDTAVYYCVRDPYDSYDQGYRLWGQGTLVTVSS

SEQ ID NO: 119 - protein, 120 aa, Artificial Sequence
  Description of Artificial Sequence: Synthetic polypeptide
EVQVLESGGGLVQPGGSLRLSCAVSGFSLSSHAMGWVRQAPGKGLEWIGIIGSSDRTYYASWAKGRFTISKDNSKNTVYLQINSPRAEDTAVYYCVRDPYDSYDSGYRLWGQGTLVTVSS

SEQ ID NO: 120 - protein, 120 aa, Artificial Sequence
  Description of Artificial Sequence: Synthetic polypeptide
EVQVLESGGGLVQPGGSLRLSCAVSGFSLSSHAMGWVRQAPGKGLEWIGIIGSSDRTYYASWAKGRFTISKDNSKNTVYLQINSPRAEDTAVYYCVRDPYDSYDNAYRLWGQGTLVTVSS